SEQUENCE LISTING (178 sequences)

SEQ ID NO: 1 - protein, 598 aa, Artificial Sequence
  42 Fc
MWPLWLCWALWVLPLAGPGAALTEEQLLGSLLRQLQLSEVPVLDRADMEKLVIPAHVRAQYVVLLRRSHGDRSGGKGFSQSFREVAGRFLASEASTHLLVFGMEQRLPPNSELVQAVLRLFQEPVPKAALHGHGRLSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFLGPRQCIASETASLPMIVSIKEGGRTRPQVVSLPNMRVQKCSCASDGALVPRRLQPEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 542 aa, Artificial Sequence
  34 Fc
MWPLWLCWALWVLPLAGPGAAFSQSFREVAGRFLASEASTHLLVFGMEQRLPPNSELVQAVLRLFQEPVPKAALHGHGRLSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFLGPRQCIASETASLPMIVSIKEGGRTRPQVVSLPNMRVQKCSCASDGALVPRRLQPEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 484 aa, Artificial Sequence
  28 Fc
MWPLWLCWALWVLPLAGPGAALSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFLGPRQCIASETASLPMIVSIKEGGRTRPQVVSLPNMRVQKCSCASDGALVPRRLQPEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 614 aa, Artificial Sequence
  42 LFc
MWPLWLCWALWVLPLAGPGAALTEEQLLGSLLRQLQLSEVPVLDRADMEKLVIPAHVRAQYVVLLRRSHGDRSGGKGFSQSFREVAGRFLASEASTHLLVFGMEQRLPPNSELVQAVLRLFQEPVPKAALHGHGRLSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFLGPRQCIASETASLPMIVSIKEGGRTRPQVVSLPNMRVQKCSCASDGALVPRRLQPSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 558 aa, Artificial Sequence
  34 LFc
MWPLWLCWALWVLPLAGPGAAFSQSFREVAGRFLASEASTHLLVFGMEQRLPPNSELVQAVLRLFQEPVPKAALHGHGRLSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFLGPRQCIASETASLPMIVSIKEGGRTRPQVVSLPNMRVQKCSCASDGALVPRRLQPSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 500 aa, Artificial Sequence
  28 LFc
MWPLWLCWALWVLPLAGPGAALSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFLGPRQCIASETASLPMIVSIKEGGRTRPQVVSLPNMRVQKCSCASDGALVPRRLQPSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 596 aa, Artificial Sequence
  Fc 42
MGWSCIILFLVATATGVHSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKLTEEQLLGSLLRQLQLSEVPVLDRADMEKLVIPAHVRAQYVVLLRRSHGDRSRGKRFSQSFREVAGRFLASEASTHLLVFGMEQRLPPNSELVQAVLRLFQEPVPKAALHRHGRLSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFLGPRQCIASETASLPMIVSIKEGGRTRPQVVSLPNMRVQKCSCASDGALVPRRLQP

SEQ ID NO: 8 - protein, 540 aa, Artificial Sequence
  Fc 34
MGWSCIILFLVATATGVHSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKFSQSFREVAGRFLASEASTHLLVFGMEQRLPPNSELVQAVLRLFQEPVPKAALHRHGRLSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFLGPRQCIASETASLPMIVSIKEGGRTRPQVVSLPNMRVQKCSCASDGALVPRRLQP

SEQ ID NO: 9 - protein, 482 aa, Artificial Sequence
  Fc 28
MGWSCIILFLVATATGVHSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKLSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFLGPRQCIASETASLPMIVSIKEGGRTRPQVVSLPNMRVQKCSCASDGALVPRRLQP

SEQ ID NO: 10 - protein, 951 aa, Artificial Sequence
  42 HSA
MWPLWLCWALWVLPLAGPGAALTEEQLLGSLLRQLQLSEVPVLDRADMEKLVIPAHVRAQYVVLLRRSHGDRSGGKGFSQSFREVAGRFLASEASTHLLVFGMEQRLPPNSELVQAVLRLFQEPVPKAALHGHGRLSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFLGPRQCIASETASLPMIVSIKEGGRTRPQVVSLPNMRVQKCSCASDGALVPRRLQPDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 11 - protein, 895 aa, Artificial Sequence
  34 HSA
MWPLWLCWALWVLPLAGPGAAFSQSFREVAGRFLASEASTHLLVFGMEQRLPPNSELVQAVLRLFQEPVPKAALHGHGRLSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFLGPRQCIASETASLPMIVSIKEGGRTRPQVVSLPNMRVQKCSCASDGALVPRRLQPDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 12 - protein, 837 aa, Artificial Sequence
  28 HSA
MWPLWLCWALWVLPLAGPGAALSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFLGPRQCIASETASLPMIVSIKEGGRTRPQVVSLPNMRVQKCSCASDGALVPRRLQPDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 13 - DNA, 36 bp, Artificial Sequence
  L1_F
aattaagcttgccaccatgtggcctctgtggctctg

SEQ ID NO: 14 - DNA, 24 bp, Artificial Sequence
  L2_R
aggttgcaatcgtcttgggacaag

SEQ ID NO: 15 - DNA, 45 bp, Artificial Sequence
  L3_F
cttgtcccaagacgattgcaacctgagcccaaatcttgtgacaaa

SEQ ID NO: 16 - DNA, 31 bp, Artificial Sequence
  L4_R
aattctcgagtcatttacccggagacaggga

SEQ ID NO: 17 - DNA, 87 bp, Artificial Sequence
  L5_F
gtcccaagacgattgcaacctagcggcggtggcggttctggcggtggtggaagtggcggtggcgggtctgagcccaaatcttgtgac

SEQ ID NO: 18 - DNA, 42 bp, Artificial Sequence
  L6_F
ctctccctgtctccgggtaaactcaccgaagagcagttgttg

SEQ ID NO: 19 - DNA, 42 bp, Artificial Sequence
  L7_F
ctctccctgtctccgggtaaattcagccaatcattccgtgag

SEQ ID NO: 20 - DNA, 42 bp, Artificial Sequence
  L8_F
ctctccctgtctccgggtaaactctccccccgaagcgcccag

SEQ ID NO: 21 - DNA, 43 bp, Artificial Sequence
  L9_R
gaaggcacagctcgagtcaaggttgcaatcgtcttgggacaag

SEQ ID NO: 22 - DNA, 45 bp, Artificial Sequence
  L10_F
gtcccaagacgattgcaacctgacgctcacaagtctgaagttgct

SEQ ID NO: 23 - DNA, 48 bp, Artificial Sequence
  L11_R
agatccacgcggaaccagctcgagtcacagccccaaagcggcctgtga

SEQ ID NO: 24 - DNA, 21 bp, Artificial Sequence
  L12_F
actctagaggatcgaaccctt

SEQ ID NO: 25 - DNA, 54 bp, Artificial Sequence
  L12_R
gctggcaactagaaggcacagctcgagtcatttacccggagacagggagaggct

SEQ ID NO: 26 - DNA, 42 bp, Artificial Sequence
  L13_F
ggggccgctctcacagrggagcagktgctcgggtcattgttg

SEQ ID NO: 27 - DNA, 42 bp, Artificial Sequence
  L13_R
caacaatgacccgagcamctgctccyctgtgagagcggcccc

SEQ ID NO: 28 - DNA, 33 bp, Artificial Sequence
  L14_F
ctcgggtcattgttgaggcagcttcagctttcc

SEQ ID NO: 29 - DNA, 33 bp, Artificial Sequence
  L14_R
ggaaagctgaagctgcctcaacaatgacccgag

SEQ ID NO: 30 - DNA, 33 bp, Artificial Sequence
  L15_F
cttcagctttccgaagtgcccgtcctggacaag

SEQ ID NO: 31 - DNA, 33 bp, Artificial Sequence
  L15_R
cttgtccaggacgggcacttcggaaagctgaag

SEQ ID NO: 32 - DNA, 42 bp, Artificial Sequence
  L16_F
ccacccgtcctggacarggccgatrtggaggggctcgttatc

SEQ ID NO: 33 - DNA, 42 bp, Artificial Sequence
  L16_R
gataacgagcccctccayatcggccytgtccaggacgggtgg

SEQ ID NO: 34 - DNA, 33 bp, Artificial Sequence
  L17_F
aaggccgatgtcgagaagctcgttatcccatcc

SEQ ID NO: 35 - DNA, 33 bp, Artificial Sequence
  L17_R
ggatgggataacgagcttctcgacatcggcctt

SEQ ID NO: 36 - DNA, 33 bp, Artificial Sequence
  L18_F
gggctcgttatcccagcccatgtccgggctcag

SEQ ID NO: 37 - DNA, 33 bp, Artificial Sequence
  L18_R
ctgagcccggacatgggctgggataacgagccc

SEQ ID NO: 38 - DNA, 33 bp, Artificial Sequence
  L19_F
cgggctcagtacgttgtgttgctgcaacattca

SEQ ID NO: 39 - DNA, 33 bp, Artificial Sequence
  L19_R
tgaatgttgcagcaacacaacgtactgagcccg

SEQ ID NO: 40 - DNA, 36 bp, Artificial Sequence
  L20_F
tacgttgccttgctgagacggtcacacgctagtaga

SEQ ID NO: 41 - DNA, 36 bp, Artificial Sequence
  L20_R
tctactagcgtgtgaccgtctcagcaaggcaacgta

SEQ ID NO: 42 - DNA, 36 bp, Artificial Sequence
  L21_F
ctgcaacattcacacgsakmcagaagtggaggcaag

SEQ ID NO: 43 - DNA, 36 bp, Artificial Sequence
  L21_R
cttgcctccacttctgkmtscgtgtgaatgttgcag

SEQ ID NO: 44 - DNA, 36 bp, Artificial Sequence
  L22_F
tacgttgccttgctgcrgcrctcacacgctagtaga

SEQ ID NO: 45 - DNA, 36 bp, Artificial Sequence
  L22_R
tctactagcgtgtgagygcygcagcaaggcaacgta

SEQ ID NO: 46 - DNA, 33 bp, Artificial Sequence
  L23_F
ggggccgccctcaccggcgagcagttgttgggc

SEQ ID NO: 47 - DNA, 33 bp, Artificial Sequence
  L23_R
gcccaacaactgctcgccggtgagggcggcccc

SEQ ID NO: 48 - DNA, 42 bp, Artificial Sequence
  L24_F
cgcgcccaatatgtggycctgctccrgcgatctcacggagat

SEQ ID NO: 49 - DNA, 42 bp, Artificial Sequence
  L24_R
atctccgtgagatcgcyggagcaggrccacatattgggcgcg

SEQ ID NO: 50 - DNA, 45 bp, Artificial Sequence
  L25_F
cgcgcccaatatgtggccctgctccrgcactctcacggagatagg

SEQ ID NO: 51 - DNA, 45 bp, Artificial Sequence
  L25_R
cctatctccgtgagagtgcyggagcagggccacatattgggcgcg

SEQ ID NO: 52 - DNA, 48 bp, Artificial Sequence
  L26_F
aaggccgatgtcgagaagctcgttatcccagcccatgtccgggctcag

SEQ ID NO: 53 - DNA, 48 bp, Artificial Sequence
  L26_R
ctgagcccggacatgggctgggataacgagcttctcgacatcggcctt

SEQ ID NO: 54 - DNA, 42 bp, Artificial Sequence
  L27_F
aaggctgccctgcaccggcatggtcgtctctccccccgaagc

SEQ ID NO: 55 - DNA, 42 bp, Artificial Sequence
  L27_R
gcttcggggggagagacgaccatgccggtgcagggcagcctt

SEQ ID NO: 56 - DNA, 33 bp, Artificial Sequence
  L28_F
ctgcacggccatggtggcctctccccccgaagc

SEQ ID NO: 57 - DNA, 33 bp, Artificial Sequence
  L28_R
gcttcggggggagaggccaccatggccgtgcag

SEQ ID NO: 58 - DNA, 33 bp, Artificial Sequence
  L29_F
aggagcaggggcaaggycttcagccaatcattc

SEQ ID NO: 59 - DNA, 33 bp, Artificial Sequence
  L29_R
gaatgattggctgaagrccttgcccctgctcct

SEQ ID NO: 60 - DNA, 33 bp, Artificial Sequence
  L30_F
ctgcaccggcatggtgycctctccccccgaagc

SEQ ID NO: 61 - DNA, 33 bp, Artificial Sequence
  L30_R
gcttcggggggagaggrcaccatgccggtgcag

SEQ ID NO: 62 - DNA, 33 bp, Artificial Sequence
  L31_F
ctgcaccggcatggtggcctctccccccgaagc

SEQ ID NO: 63 - DNA, 33 bp, Artificial Sequence
  L31_R
gcttcggggggagaggccaccatgccggtgcag

SEQ ID NO: 64 - DNA, 33 bp, Artificial Sequence
  L32_F
  misc_feature (16)..(17): n is a, c, g, or t
ctccttctgcaagtgnnkgtccagcgcgaacac

SEQ ID NO: 65 - DNA, 33 bp, Artificial Sequence
  L32_R
  misc_feature (17)..(18): n is a, c, g, or t
gtgttcgcgctggacmnncacttgcagaaggag

SEQ ID NO: 66 - DNA, 33 bp, Artificial Sequence
  L33_F
  misc_feature (16)..(17): n is a, c, g, or t
ctggtgcgattcgctnnkcagggtgcaccagcc

SEQ ID NO: 67 - DNA, 33 bp, Artificial Sequence
  L33_R
  misc_feature (17)..(18): n is a, c, g, or t
ggctggtgcaccctgmnnagcgaatcgcaccag

SEQ ID NO: 68 - DNA, 42 bp, Artificial Sequence
  L34_F
ttcaactggccctttgasggacctcggcaatgtattgccagc

SEQ ID NO: 69 - DNA, 42 bp, Artificial Sequence
  L34_R
gctggcaatacattgccgaggtccstcaaagggccagttgaa

SEQ ID NO: 70 - DNA, 42 bp, Artificial Sequence
  L35_F
ttcaactggccctttctcggagascggcaatgtattgccagc

SEQ ID NO: 71 - DNA, 42 bp, Artificial Sequence
  L35_R
gctggcaatacattgccgstctccgagaaagggccagttgaa

SEQ ID NO: 72 - DNA, 42 bp, Artificial Sequence
  L36_F
ttcaactggccctttctcggatcccggcaatgtattgccagc

SEQ ID NO: 73 - DNA, 42 bp, Artificial Sequence
  L36_R
gctggcaatacattgccgggatccgagaaagggccagttgaa

SEQ ID NO: 74 - DNA, 42 bp, Artificial Sequence
  L37_F
ttcaactggccctttctcggacctmagcaatgtattgccagc

SEQ ID NO: 75 - DNA, 42 bp, Artificial Sequence
  L37_R
gctggcaatacattgctkaggtccgagaaagggccagttgaa

SEQ ID NO: 76 - DNA, 42 bp, Artificial Sequence
  L38_F
gcctctgacggggctgasgtcccaagacgattgcaaccttct

SEQ ID NO: 77 - DNA, 42 bp, Artificial Sequence
  L38_R
agaaggttgcaatcgtcttgggacstcagccccgtcagaggc

SEQ ID NO: 78 - DNA, 42 bp, Artificial Sequence
  L39_F
gcctctgacggggctcttgtcgasagacgattgcaaccttct

SEQ ID NO: 79 - DNA, 42 bp, Artificial Sequence
  L39_R
agaaggttgcaatcgtctstcgacaagagccccgtcagaggc

SEQ ID NO: 80 - DNA, 42 bp, Artificial Sequence
  L40_F
gcctctgacggggctcttgtctccagacgattgcaaccttct

SEQ ID NO: 81 - DNA, 42 bp, Artificial Sequence
  L40_R
agaaggttgcaatcgtctggagacaagagccccgtcagaggc

SEQ ID NO: 82 - DNA, 42 bp, Artificial Sequence
  L41_F
gcctctgacggggctcttgtcccamagcgattgcaaccttct

SEQ ID NO: 83 - DNA, 42 bp, Artificial Sequence
  L41_R
agaaggttgcaatcgctktgggacaagagccccgtcagaggc

SEQ ID NO: 84 - DNA, 42 bp, Artificial Sequence
  L42_F
gcctctgacggggctcttgtcgaccagcgattgcaaccttct

SEQ ID NO: 85 - DNA, 42 bp, Artificial Sequence
  L42_R
agaaggttgcaatcgctggtcgacaagagccccgtcagaggc

SEQ ID NO: 86 - protein, 614 aa, Artificial Sequence
  42 LFc(V63A)
MWPLWLCWALWVLPLAGPGAALTEEQLLGSLLRQLQLSEVPVLDRADMEKLVIPAHVRAQYVALLRRSHGDRSGGKGFSQSFREVAGRFLASEASTHLLVFGMEQRLPPNSELVQAVLRLFQEPVPKAALHGHGRLSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFLGPRQCIASETASLPMIVSIKEGGRTRPQVVSLPNMRVQKCSCASDGALVPRRLQPSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 87 - protein, 614 aa, Artificial Sequence
  42 LFc(R66Q)
MWPLWLCWALWVLPLAGPGAALTEEQLLGSLLRQLQLSEVPVLDRADMEKLVIPAHVRAQYVVLLQRSHGDRSGGKGFSQSFREVAGRFLASEASTHLLVFGMEQRLPPNSELVQAVLRLFQEPVPKAALHGHGRLSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFLGPRQCIASETASLPMIVSIKEGGRTRPQVVSLPNMRVQKCSCASDGALVPRRLQPSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 88 - protein, 614 aa, Artificial Sequence
  42 LFc(V63A/R66Q)
MWPLWLCWALWVLPLAGPGAALTEEQLLGSLLRQLQLSEVPVLDRADMEKLVIPAHVRAQYVALLQRSHGDRSGGKGFSQSFREVAGRFLASEASTHLLVFGMEQRLPPNSELVQAVLRLFQEPVPKAALHGHGRLSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFLGPRQCIASETASLPMIVSIKEGGRTRPQVVSLPNMRVQKCSCASDGALVPRRLQPSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 89 - protein, 614 aa, Artificial Sequence
  42 LFc(V63A/R67H)
MWPLWLCWALWVLPLAGPGAALTEEQLLGSLLRQLQLSEVPVLDRADMEKLVIPAHVRAQYVALLRHSHGDRSGGKGFSQSFREVAGRFLASEASTHLLVFGMEQRLPPNSELVQAVLRLFQEPVPKAALHGHGRLSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFLGPRQCIASETASLPMIVSIKEGGRTRPQVVSLPNMRVQKCSCASDGALVPRRLQPSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 90 - protein, 614 aa, Artificial Sequence
  42 LFc(V63A/R66Q/R67H)
MWPLWLCWALWVLPLAGPGAALTEEQLLGSLLRQLQLSEVPVLDRADMEKLVIPAHVRAQYVALLQHSHGDRSGGKGFSQSFREVAGRFLASEASTHLLVFGMEQRLPPNSELVQAVLRLFQEPVPKAALHGHGRLSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFLGPRQCIASETASLPMIVSIKEGGRTRPQVVSLPNMRVQKCSCASDGALVPRRLQPSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 91 - protein, 614 aa, Artificial Sequence
  LFB 42 LFc
MWPLWLCWALWVLPLAGPGAALTGEQLLGSLLRQLQLKEVPTLDRADMEELVIPTHVRAQYVALLQRSHGDRSGGKGFSQSFREVAGRFLASEASTHLLVFGMEQRLPPNSELVQAVLRLFQEPVPKAALHGHGRLSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFLGPRQCIASETASLPMIVSIKEGGRTRPQVVSLPNMRVQKCSCASDGALVPRRLQPSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 92 - protein, 614 aa, Artificial Sequence
  42 LFc V1
MWPLWLCWALWVLPLAGPGAALTEEQLLGSLLRQLQLSEVPVLDRADMEKLVIPAHVRAQYVVLLRRSHGDRSGGKGFSQSFREVAGRFLASEASTHLLVFGMEQRLPPNSELVQAVLRLFQEPVPKAALHRHGRLSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFLGPRQCIASETASLPMIVSIKEGGRTRPQVVSLPNMRVQKCSCASDGALVPRRLQPSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 93 - protein, 614 aa, Artificial Sequence
  42 LFc V2
MWPLWLCWALWVLPLAGPGAALTEEQLLGSLLRQLQLSEVPVLDRADMEKLVIPAHVRAQYVVLLRRSHGDRSGGKGFSQSFREVAGRFLASEASTHLLVFGMEQRLPPNSELVQAVLRLFQEPVPKAALHRHGGLSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFLGPRQCIASETASLPMIVSIKEGGRTRPQVVSLPNMRVQKCSCASDGALVPRRLQPSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 94 - protein, 614 aa, Artificial Sequence
  42 LFc V3
MWPLWLCWALWVLPLAGPGAALTEEQLLGSLLRQLQLSEVPVLDRADMEKLVIPAHVRAQYVVLLRRSHGDRSGGKGFSQSFREVAGRFLASEASTHLLVFGMEQRLPPNSELVQAVLRLFQEPVPKAALHRHERLSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFLGPRQCIASETASLPMIVSIKEGGRTRPQVVSLPNMRVQKCSCASDGALVPRRLQPSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 95 - protein, 614 aa, Artificial Sequence
  42 LFc V4
MWPLWLCWALWVLPLAGPGAALTEEQLLGSLLRQLQLSEVPVLDRADMEKLVIPAHVRAQYVVLLRRSHGDRSGGKGFSQSFREVAGRFLASEASTHLLVFGMEQRLPPNSELVQAVLRLFQEPVPKAALHGHGGLSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFLGPRQCIASETASLPMIVSIKEGGRTRPQVVSLPNMRVQKCSCASDGALVPRRLQPSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 96 - protein, 614 aa, Artificial Sequence
  42 LFc V5
MWPLWLCWALWVLPLAGPGAALTEEQLLGSLLRQLQLSEVPVLDRADMEKLVIPAHVRAQYVVLLRRSHGDRSRGKAFSQSFREVAGRFLASEASTHLLVFGMEQRLPPNSELVQAVLRLFQEPVPKAALHGHGRLSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFLGPRQCIASETASLPMIVSIKEGGRTRPQVVSLPNMRVQKCSCASDGALVPRRLQPSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 97 - protein, 614 aa, Artificial Sequence
  42 LFc V6
MWPLWLCWALWVLPLAGPGAALTEEQLLGSLLRQLQLSEVPVLDRADMEKLVIPAHVRAQYVVLLRRSHGDRSRGKAFSQSFREVAGRFLASEASTHLLVFGMEQRLPPNSELVQAVLRLFQEPVPKAALHRHGALSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFLGPRQCIASETASLPMIVSIKEGGRTRPQVVSLPNMRVQKCSCASDGALVPRRLQPSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 98 - protein, 614 aa, Artificial Sequence
  42 LFc V7
MWPLWLCWALWVLPLAGPGAALTEEQLLGSLLRQLQLSEVPVLDRADMEKLVIPAHVRAQYVVLLRRSHGDRSRGKAFSQSFREVAGRFLASEASTHLLVFGMEQRLPPNSELVQAVLRLFQEPVPKAALHRHGVLSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFLGPRQCIASETASLPMIVSIKEGGRTRPQVVSLPNMRVQKCSCASDGALVPRRLQPSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 99 - protein, 614 aa, Artificial Sequence
  42 LFc V8
MWPLWLCWALWVLPLAGPGAALTEEQLLGSLLRQLQLSEVPVLDRADMEKLVIPAHVRAQYVVLLRRSHGDRSRGKVFSQSFREVAGRFLASEASTHLLVFGMEQRLPPNSELVQAVLRLFQEPVPKAALHGHGRLSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFLGPRQCIASETASLPMIVSIKEGGRTRPQVVSLPNMRVQKCSCASDGALVPRRLQPSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 100 - protein, 614 aa, Artificial Sequence
  42 LFc V9
MWPLWLCWALWVLPLAGPGAALTEEQLLGSLLRQLQLSEVPVLDRADMEKLVIPAHVRAQYVVLLRRSHGDRSRGKVFSQSFREVAGRFLASEASTHLLVFGMEQRLPPNSELVQAVLRLFQEPVPKAALHRHGALSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFLGPRQCIASETASLPMIVSIKEGGRTRPQVVSLPNMRVQKCSCASDGALVPRRLQPSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 101 - protein, 614 aa, Artificial Sequence
  42 LFc V10
MWPLWLCWALWVLPLAGPGAALTEEQLLGSLLRQLQLSEVPVLDRADMEKLVIPAHVRAQYVVLLRRSHGDRSRGKVFSQSFREVAGRFLASEASTHLLVFGMEQRLPPNSELVQAVLRLFQEPVPKAALHRHGVLSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFLGPRQCIASETASLPMIVSIKEGGRTRPQVVSLPNMRVQKCSCASDGALVPRRLQPSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 102 - protein, 614 aa, Artificial Sequence
  42 LFc V11
MWPLWLCWALWVLPLAGPGAALTEEQLLGSLLRQLQLSEVPVLDRADMEKLVIPAHVRAQYVVLLRRSHGDRSRHGGFSQSFREVAGRFLASEASTHLLVFGMEQRLPPNSELVQAVLRLFQEPVPKAALHRHGGLSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFLGPRQCIASETASLPMIVSIKEGGRTRPQVVSLPNMRVQKCSCASDGALVPRRLQPSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 103 - protein, 614 aa, Artificial Sequence
  42 LFc V12
MWPLWLCWALWVLPLAGPGAALTEEQLLGSLLRQLQLSEVPVLDRADMEKLVIPAHVRAQYVVLLRRSHGDRSRGKGFSQSFREVAGRFLASEASTHLLVFGMEQRLPPNSELVQAVLRLFQEPVPKAALHRHGGLSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFLGPRQCIASETASLPMIVSIKEGGRTRPQVVSLPNMRVQKCSCASDGALVPRRLQPSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 104 - protein, 614 aa, Artificial Sequence
  42 LFc V13
MWPLWLCWALWVLPLAGPGAALTEEQLLGSLLRQLQLSEVPVLDRADMEKLVIPAHVRAQYVVLLRRSHGDRSRGKAFSQSFREVAGRFLASEASTHLLVFGMEQRLPPNSELVQAVLRLFQEPVPKAALHRHGGLSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFLGPRQCIASETASLPMIVSIKEGGRTRPQVVSLPNMRVQKCSCASDGALVPRRLQPSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 105 - protein, 614 aa, Artificial Sequence
  CX196
MWPLWLCWALWVLPLAGPGAALTGEQLLGSLLRQLQLKEVPTLDRADMEELVIPTHVRAQYVALLQRSHGDRSGGKGFSQSFREVAGRFLASEASTHLLVFGMEQRLPPNSELVQAVLRLFQEPVPKAALHRHGGLSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFLGPRQCIASETASLPMIVSIKEGGRTRPQVVSLPNMRVQKCSCASDGALVPRRLQPSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 106 - protein, 614 aa, Artificial Sequence
  CX197
MWPLWLCWALWVLPLAGPGAALTGEQLLGSLLRQLQLKEVPTLDRADMEELVIPTHVRAQYVALLQRSHGDRSGGKGFSQSFREVAGRFLASEASTHLLVFGMEQRLPPNSELVQAVLRLFQEPVPKAALHRHGALSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFLGPRQCIASETASLPMIVSIKEGGRTRPQVVSLPNMRVQKCSCASDGALVPRRLQPSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 107 - protein, 614 aa, Artificial Sequence
  CX201
MWPLWLCWALWVLPLAGPGAALTEEQLLGSLLRQLQLSEVPVLDRADMEKLVIPAHVRAQYVALLQRSHGDRSGGKGFSQSFREVAGRFLASEASTHLLVFGMEQRLPPNSELVQAVLRLFQEPVPKAALHRHGGLSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFLGPRQCIASETASLPMIVSIKEGGRTRPQVVSLPNMRVQKCSCASDGALVPRRLQPSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 108 - protein, 614 aa, Artificial Sequence
  CX203
MWPLWLCWALWVLPLAGPGAALTEEQLLGSLLRQLQLSEVPVLDRADMEKLVIPAHVRAQYVALLQRSHGDRSGGKGFSQSFREVAGRFLASEASTHLLVFGMEQRLPPNSELVQAVLRLFQEPVPKAALHRHERLSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFLGPRQCIASETASLPMIVSIKEGGRTRPQVVSLPNMRVQKCSCASDGALVPRRLQPSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 109 - protein, 614 aa, Artificial Sequence
  CX206
MWPLWLCWALWVLPLAGPGAALTEEQLLGSLLRQLQLSEVPVLDRADMEKLVIPAHVRAQYVALLQRSHGDRSGGKGFSQSFREVAGRFLASEASTHLLVFGMEQRLPPNSELVQAVLRLFQEPVPKAALHRHGGLSPRSAQARVTVEWLRVRDDGSNRTSLIDSRLVSVHESGWKAFDVTEAVNFWQQLSRPRQPLLLQVTVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFLGPRQCIASETASLPMIVSIKEGGRTRPQVVSLPNMRVQKCSCASDGALVPRRLQPSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 110 - protein, 614 aa, Artificial Sequence
  CX207
MWPLWLCWALWVLPLAGPGAALTEEQLLGSLLRQLQLSEVPVLDRADMEKLVIPAHVRAQYVALLQRSHGDRSGGKGFSQSFREVAGRFLASEASTHLLVFGMEQRLPPNSELVQAVLRLFQEPVPKAALHRHGGLSPRSAQARVTVEWLRVRDDGSNRTSLIDSRLVSVHESGWKAFDVTEAVNFWQQLSRPRQPLLLQVSVQREHLGPLASGAHKLVRFAGQGAPAGLGEPQLELHTLDLRDYGAQGDCDPEAPMTEGTRCCRQEMYIDLQGMKWAKNWVLEPPGFLAYECVGTCQQPPEALAFNWPFLGPRQCIASETASLPMIVSIKEGGRTRPQVVSLPNMRVQKCSCASDGALVPRRLQPSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 111 - protein, 614 aa, Artificial Sequence
  CX208
MWPLWLCWALWVLPLAGPGAALTEEQLLGSLLRQLQLSEVPVLDRADMEKLVIPAHVRAQYVALLQRSHGDRSGGKGFSQSFREVAGRFLASEASTHLLVFGMEQRLPPNSELVQAVLRLFQEPVPKAALHRHGGLSPRSAQARVTVEWLRVRDDGSNRTSLIDSRLVSVHESGWKAFDVTEAVNFWQQLSRPRQPLLLQVTVQREHLGPLASGAHKLVRFAGQGAPAGLGEPQLELHTLDLRDYGAQGDCDPEAPMTEGTRCCRQEMYIDLQGMKWAKNWVLEPPGFLAYECVGTCQQPPEALAFNWPFLGPRQCIASETASLPMIVSIKEGGRTRPQVVSLPNMRVQKCSCASDGALVPRRLQPSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 112 - protein, 615 aa, Artificial Sequence
  CX201(L311D)
MDMRVPAQLLGLLLLWFPGSRCLTEEQLLGSLLRQLQLSEVPVLDRADMEKLVIPAHVRAQYVALLQRSHGDRSGGKGFSQSFREVAGRFLASEASTHLLVFGMEQRLPPNSELVQAVLRLFQEPVPKAALHRHGGLSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFDGPRQCIASETASLPMIVSIKEGGRTRPQVVSLPNMRVQKCSCASDGALVPRRLQPSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 113 - protein, 615 aa, Artificial Sequence
  CX201(L311E)
MDMRVPAQLLGLLLLWFPGSRCLTEEQLLGSLLRQLQLSEVPVLDRADMEKLVIPAHVRAQYVALLQRSHGDRSGGKGFSQSFREVAGRFLASEASTHLLVFGMEQRLPPNSELVQAVLRLFQEPVPKAALHRHGGLSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFEGPRQCIASETASLPMIVSIKEGGRTRPQVVSLPNMRVQKCSCASDGALVPRRLQPSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 114 - protein, 615 aa, Artificial Sequence
  CX201(P313D)
MDMRVPAQLLGLLLLWFPGSRCLTEEQLLGSLLRQLQLSEVPVLDRADMEKLVIPAHVRAQYVALLQRSHGDRSGGKGFSQSFREVAGRFLASEASTHLLVFGMEQRLPPNSELVQAVLRLFQEPVPKAALHRHGGLSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFLGDRQCIASETASLPMIVSIKEGGRTRPQVVSLPNMRVQKCSCASDGALVPRRLQPSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 115 - protein, 615 aa, Artificial Sequence
  CX201(P313E)
MDMRVPAQLLGLLLLWFPGSRCLTEEQLLGSLLRQLQLSEVPVLDRADMEKLVIPAHVRAQYVALLQRSHGDRSGGKGFSQSFREVAGRFLASEASTHLLVFGMEQRLPPNSELVQAVLRLFQEPVPKAALHRHGGLSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFLGERQCIASETASLPMIVSIKEGGRTRPQVVSLPNMRVQKCSCASDGALVPRRLQPSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 116 - protein, 615 aa, Artificial Sequence
  CX201(P313S)
MDMRVPAQLLGLLLLWFPGSRCLTEEQLLGSLLRQLQLSEVPVLDRADMEKLVIPAHVRAQYVALLQRSHGDRSGGKGFSQSFREVAGRFLASEASTHLLVFGMEQRLPPNSELVQAVLRLFQEPVPKAALHRHGGLSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFLGSRQCIASETASLPMIVSIKEGGRTRPQVVSLPNMRVQKCSCASDGALVPRRLQPSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 117 - protein, 615 aa, Artificial Sequence
  CX201(R314K)
MDMRVPAQLLGLLLLWFPGSRCLTEEQLLGSLLRQLQLSEVPVLDRADMEKLVIPAHVRAQYVALLQRSHGDRSGGKGFSQSFREVAGRFLASEASTHLLVFGMEQRLPPNSELVQAVLRLFQEPVPKAALHRHGGLSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFLGPKQCIASETASLPMIVSIKEGGRTRPQVVSLPNMRVQKCSCASDGALVPRRLQPSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 118 - protein, 615 aa, Artificial Sequence
  CX201(R314Q)
MDMRVPAQLLGLLLLWFPGSRCLTEEQLLGSLLRQLQLSEVPVLDRADMEKLVIPAHVRAQYVALLQRSHGDRSGGKGFSQSFREVAGRFLASEASTHLLVFGMEQRLPPNSELVQAVLRLFQEPVPKAALHRHGGLSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFLGPQQCIASETASLPMIVSIKEGGRTRPQVVSLPNMRVQKCSCASDGALVPRRLQPSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 119 - protein, 615 aa, Artificial Sequence
  CX201(L359D)
MDMRVPAQLLGLLLLWFPGSRCLTEEQLLGSLLRQLQLSEVPVLDRADMEKLVIPAHVRAQYVALLQRSHGDRSGGKGFSQSFREVAGRFLASEASTHLLVFGMEQRLPPNSELVQAVLRLFQEPVPKAALHRHGGLSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFLGPRQCIASETASLPMIVSIKEGGRTRPQVVSLPNMRVQKCSCASDGADVPRRLQPSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 120 - protein, 615 aa, Artificial Sequence
  CX201(L359E)
MDMRVPAQLLGLLLLWFPGSRCLTEEQLLGSLLRQLQLSEVPVLDRADMEKLVIPAHVRAQYVALLQRSHGDRSGGKGFSQSFREVAGRFLASEASTHLLVFGMEQRLPPNSELVQAVLRLFQEPVPKAALHRHGGLSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFLGPRQCIASETASLPMIVSIKEGGRTRPQVVSLPNMRVQKCSCASDGAEVPRRLQPSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 121 - protein, 615 aa, Artificial Sequence
  CX201(P361D)
MDMRVPAQLLGLLLLWFPGSRCLTEEQLLGSLLRQLQLSEVPVLDRADMEKLVIPAHVRAQYVALLQRSHGDRSGGKGFSQSFREVAGRFLASEASTHLLVFGMEQRLPPNSELVQAVLRLFQEPVPKAALHRHGGLSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFLGPRQCIASETASLPMIVSIKEGGRTRPQVVSLPNMRVQKCSCASDGALVDRRLQPSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 122 - protein, 615 aa, Artificial Sequence
  CX201(P361E)
MDMRVPAQLLGLLLLWFPGSRCLTEEQLLGSLLRQLQLSEVPVLDRADMEKLVIPAHVRAQYVALLQRSHGDRSGGKGFSQSFREVAGRFLASEASTHLLVFGMEQRLPPNSELVQAVLRLFQEPVPKAALHRHGGLSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFLGPRQCIASETASLPMIVSIKEGGRTRPQVVSLPNMRVQKCSCASDGALVERRLQPSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 123 - protein, 615 aa, Artificial Sequence
  CX201(P361S)
MDMRVPAQLLGLLLLWFPGSRCLTEEQLLGSLLRQLQLSEVPVLDRADMEKLVIPAHVRAQYVALLQRSHGDRSGGKGFSQSFREVAGRFLASEASTHLLVFGMEQRLPPNSELVQAVLRLFQEPVPKAALHRHGGLSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFLGPRQCIASETASLPMIVSIKEGGRTRPQVVSLPNMRVQKCSCASDGALVSRRLQPSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 124 - protein, 615 aa, Artificial Sequence
  CX201(R362K)
MDMRVPAQLLGLLLLWFPGSRCLTEEQLLGSLLRQLQLSEVPVLDRADMEKLVIPAHVRAQYVALLQRSHGDRSGGKGFSQSFREVAGRFLASEASTHLLVFGMEQRLPPNSELVQAVLRLFQEPVPKAALHRHGGLSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFLGPRQCIASETASLPMIVSIKEGGRTRPQVVSLPNMRVQKCSCASDGALVPKRLQPSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 125 - protein, 615 aa, Artificial Sequence
  CX201(R362Q)
MDMRVPAQLLGLLLLWFPGSRCLTEEQLLGSLLRQLQLSEVPVLDRADMEKLVIPAHVRAQYVALLQRSHGDRSGGKGFSQSFREVAGRFLASEASTHLLVFGMEQRLPPNSELVQAVLRLFQEPVPKAALHRHGGLSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFLGPRQCIASETASLPMIVSIKEGGRTRPQVVSLPNMRVQKCSCASDGALVPQRLQPSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 126 - protein, 615 aa, Artificial Sequence
  CX201(L311D/R362Q)
MDMRVPAQLLGLLLLWFPGSRCLTEEQLLGSLLRQLQLSEVPVLDRADMEKLVIPAHVRAQYVALLQRSHGDRSGGKGFSQSFREVAGRFLASEASTHLLVFGMEQRLPPNSELVQAVLRLFQEPVPKAALHRHGGLSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFDGPRQCIASETASLPMIVSIKEGGRTRPQVVSLPNMRVQKCSCASDGALVPQRLQPSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 127 - protein, 615 aa, Artificial Sequence
  CX201(L311E/R362Q)
MDMRVPAQLLGLLLLWFPGSRCLTEEQLLGSLLRQLQLSEVPVLDRADMEKLVIPAHVRAQYVALLQRSHGDRSGGKGFSQSFREVAGRFLASEASTHLLVFGMEQRLPPNSELVQAVLRLFQEPVPKAALHRHGGLSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFEGPRQCIASETASLPMIVSIKEGGRTRPQVVSLPNMRVQKCSCASDGALVPQRLQPSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 128 - protein, 615 aa, Artificial Sequence
  CX201(L311D/P361D/R362Q)
MDMRVPAQLLGLLLLWFPGSRCLTEEQLLGSLLRQLQLSEVPVLDRADMEKLVIPAHVRAQYVALLQRSHGDRSGGKGFSQSFREVAGRFLASEASTHLLVFGMEQRLPPNSELVQAVLRLFQEPVPKAALHRHGGLSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFDGPRQCIASETASLPMIVSIKEGGRTRPQVVSLPNMRVQKCSCASDGALVDQRLQPSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 129 - protein, 615 aa, Artificial Sequence
  CX201(L311E/P361D/R362Q)
MDMRVPAQLLGLLLLWFPGSRCLTEEQLLGSLLRQLQLSEVPVLDRADMEKLVIPAHVRAQYVALLQRSHGDRSGGKGFSQSFREVAGRFLASEASTHLLVFGMEQRLPPNSELVQAVLRLFQEPVPKAALHRHGGLSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFEGPRQCIASETASLPMIVSIKEGGRTRPQVVSLPNMRVQKCSCASDGALVDQRLQPSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 130 - protein, 16 aa, Artificial Sequence
  Linker
SGGGGSGGGGSGGGGS

SEQ ID NO: 131 - protein, 366 aa, Artificial Sequence
  Human Lefty A
MWPLWLCWALWVLPLAGPGAALTEEQLLGSLLRQLQLSEVPVLDRADMEKLVIPAHVRAQYVVLLRRSHGDRSRGKRFSQSFREVAGRFLASEASTHLLVFGMEQRLPPNSELVQAVLRLFQEPVPKAALHRHGRLSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFLGPRQCIASETASLPMIVSIKEGGRTRPQVVSLPNMRVQKCSCASDGALVPRRLQP

SEQ ID NO: 132 - protein, 22 aa, Artificial Sequence
  signal peptide
MDMRVPAQLLGLLLLWFPGSRC

SEQ ID NO: 133 - protein, 615 aa, Artificial Sequence
  CX201s
MDMRVPAQLLGLLLLWFPGSRCLTEEQLLGSLLRQLQLSEVPVLDRADMEKLVIPAHVRAQYVALLQRSHGDRSGGKGFSQSFREVAGRFLASEASTHLLVFGMEQRLPPNSELVQAVLRLFQEPVPKAALHRHGGLSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFLGPRQCIASETASLPMIVSIKEGGRTRPQVVSLPNMRVQKCSCASDGALVPRRLQPSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 134 - protein, 593 aa, Artificial Sequence
  42 LFc(V63A)
LTEEQLLGSLLRQLQLSEVPVLDRADMEKLVIPAHVRAQYVALLRRSHGDRSGGKGFSQSFREVAGRFLASEASTHLLVFGMEQRLPPNSELVQAVLRLFQEPVPKAALHGHGRLSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFLGPRQCIASETASLPMIVSIKEGGRTRPQVVSLPNMRVQKCSCASDGALVPRRLQPSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 135 - protein, 593 aa, Artificial Sequence
  42 LFc(R66Q)
LTEEQLLGSLLRQLQLSEVPVLDRADMEKLVIPAHVRAQYVVLLQRSHGDRSGGKGFSQSFREVAGRFLASEASTHLLVFGMEQRLPPNSELVQAVLRLFQEPVPKAALHGHGRLSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFLGPRQCIASETASLPMIVSIKEGGRTRPQVVSLPNMRVQKCSCASDGALVPRRLQPSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 136 - protein, 593 aa, Artificial Sequence
  42 LFc(V63A/R66Q)
LTEEQLLGSLLRQLQLSEVPVLDRADMEKLVIPAHVRAQYVALLQRSHGDRSGGKGFSQSFREVAGRFLASEASTHLLVFGMEQRLPPNSELVQAVLRLFQEPVPKAALHGHGRLSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFLGPRQCIASETASLPMIVSIKEGGRTRPQVVSLPNMRVQKCSCASDGALVPRRLQPSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 137 - protein, 593 aa, Artificial Sequence
  42 LFc(V63A/R67H)
LTEEQLLGSLLRQLQLSEVPVLDRADMEKLVIPAHVRAQYVALLRHSHGDRSGGKGFSQSFREVAGRFLASEASTHLLVFGMEQRLPPNSELVQAVLRLFQEPVPKAALHGHGRLSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFLGPRQCIASETASLPMIVSIKEGGRTRPQVVSLPNMRVQKCSCASDGALVPRRLQPSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 138 - protein, 593 aa, Artificial Sequence
  42 LFc(V63A/R66Q/R67H)
LTEEQLLGSLLRQLQLSEVPVLDRADMEKLVIPAHVRAQYVALLQHSHGDRSGGKGFSQSFREVAGRFLASEASTHLLVFGMEQRLPPNSELVQAVLRLFQEPVPKAALHGHGRLSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFLGPRQCIASETASLPMIVSIKEGGRTRPQVVSLPNMRVQKCSCASDGALVPRRLQPSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 139 - protein, 593 aa, Artificial Sequence
  LFB 42 LFc
LTGEQLLGSLLRQLQLKEVPTLDRADMEELVIPTHVRAQYVALLQRSHGDRSGGKGFSQSFREVAGRFLASEASTHLLVFGMEQRLPPNSELVQAVLRLFQEPVPKAALHGHGRLSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFLGPRQCIASETASLPMIVSIKEGGRTRPQVVSLPNMRVQKCSCASDGALVPRRLQPSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 140 - protein, 593 aa, Artificial Sequence
  42 LFc V1
LTEEQLLGSLLRQLQLSEVPVLDRADMEKLVIPAHVRAQYVVLLRRSHGDRSGGKGFSQSFREVAGRFLASEASTHLLVFGMEQRLPPNSELVQAVLRLFQEPVPKAALHRHGRLSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFLGPRQCIASETASLPMIVSIKEGGRTRPQVVSLPNMRVQKCSCASDGALVPRRLQPSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 141 - protein, 593 aa, Artificial Sequence
  42 LFc V2
LTEEQLLGSLLRQLQLSEVPVLDRADMEKLVIPAHVRAQYVVLLRRSHGDRSGGKGFSQSFREVAGRFLASEASTHLLVFGMEQRLPPNSELVQAVLRLFQEPVPKAALHRHGGLSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFLGPRQCIASETASLPMIVSIKEGGRTRPQVVSLPNMRVQKCSCASDGALVPRRLQPSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 142 - protein, 593 aa, Artificial Sequence
  42 LFc V3
LTEEQLLGSLLRQLQLSEVPVLDRADMEKLVIPAHVRAQYVVLLRRSHGDRSGGKGFSQSFREVAGRFLASEASTHLLVFGMEQRLPPNSELVQAVLRLFQEPVPKAALHRHERLSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFLGPRQCIASETASLPMIVSIKEGGRTRPQVVSLPNMRVQKCSCASDGALVPRRLQPSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 143 - protein, 593 aa, Artificial Sequence
  42 LFc V4
LTEEQLLGSLLRQLQLSEVPVLDRADMEKLVIPAHVRAQYVVLLRRSHGDRSGGKGFSQSFREVAGRFLASEASTHLLVFGMEQRLPPNSELVQAVLRLFQEPVPKAALHGHGGLSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFLGPRQCIASETASLPMIVSIKEGGRTRPQVVSLPNMRVQKCSCASDGALVPRRLQPSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 144 - protein, 593 aa, Artificial Sequence
  42 LFc V5
LTEEQLLGSLLRQLQLSEVPVLDRADMEKLVIPAHVRAQYVVLLRRSHGDRSRGKAFSQSFREVAGRFLASEASTHLLVFGMEQRLPPNSELVQAVLRLFQEPVPKAALHGHGRLSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFLGPRQCIASETASLPMIVSIKEGGRTRPQVVSLPNMRVQKCSCASDGALVPRRLQPSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 145 - protein, 593 aa, Artificial Sequence
  42 LFc V6
LTEEQLLGSLLRQLQLSEVPVLDRADMEKLVIPAHVRAQYVVLLRRSHGDRSRGKAFSQSFREVAGRFLASEASTHLLVFGMEQRLPPNSELVQAVLRLFQEPVPKAALHRHGALSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFLGPRQCIASETASLPMIVSIKEGGRTRPQVVSLPNMRVQKCSCASDGALVPRRLQPSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 146 - protein, 593 aa, Artificial Sequence
  42 LFc V7
LTEEQLLGSLLRQLQLSEVPVLDRADMEKLVIPAHVRAQYVVLLRRSHGDRSRGKAFSQSFREVAGRFLASEASTHLLVFGMEQRLPPNSELVQAVLRLFQEPVPKAALHRHGVLSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFLGPRQCIASETASLPMIVSIKEGGRTRPQVVSLPNMRVQKCSCASDGALVPRRLQPSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 147 - protein, 593 aa, Artificial Sequence
  42 LFc V8
LTEEQLLGSLLRQLQLSEVPVLDRADMEKLVIPAHVRAQYVVLLRRSHGDRSRGKVFSQSFREVAGRFLASEASTHLLVFGMEQRLPPNSELVQAVLRLFQEPVPKAALHGHGRLSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFLGPRQCIASETASLPMIVSIKEGGRTRPQVVSLPNMRVQKCSCASDGALVPRRLQPSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 148 - protein, 593 aa, Artificial Sequence
  42 LFc V9
LTEEQLLGSLLRQLQLSEVPVLDRADMEKLVIPAHVRAQYVVLLRRSHGDRSRGKVFSQSFREVAGRFLASEASTHLLVFGMEQRLPPNSELVQAVLRLFQEPVPKAALHRHGALSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFLGPRQCIASETASLPMIVSIKEGGRTRPQVVSLPNMRVQKCSCASDGALVPRRLQPSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 149 - protein, 593 aa, Artificial Sequence
  42 LFc V10
LTEEQLLGSLLRQLQLSEVPVLDRADMEKLVIPAHVRAQYVVLLRRSHGDRSRGKVFSQSFREVAGRFLASEASTHLLVFGMEQRLPPNSELVQAVLRLFQEPVPKAALHRHGVLSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFLGPRQCIASETASLPMIVSIKEGGRTRPQVVSLPNMRVQKCSCASDGALVPRRLQPSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 150 - protein, 593 aa, Artificial Sequence
  42 LFc V11
LTEEQLLGSLLRQLQLSEVPVLDRADMEKLVIPAHVRAQYVVLLRRSHGDRSRHGGFSQSFREVAGRFLASEASTHLLVFGMEQRLPPNSELVQAVLRLFQEPVPKAALHRHGGLSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFLGPRQCIASETASLPMIVSIKEGGRTRPQVVSLPNMRVQKCSCASDGALVPRRLQPSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 151 - protein, 593 aa, Artificial Sequence
  42 LFc V12
LTEEQLLGSLLRQLQLSEVPVLDRADMEKLVIPAHVRAQYVVLLRRSHGDRSRGKGFSQSFREVAGRFLASEASTHLLVFGMEQRLPPNSELVQAVLRLFQEPVPKAALHRHGGLSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFLGPRQCIASETASLPMIVSIKEGGRTRPQVVSLPNMRVQKCSCASDGALVPRRLQPSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 152 - protein, 593 aa, Artificial Sequence
  42 LFc V13
LTEEQLLGSLLRQLQLSEVPVLDRADMEKLVIPAHVRAQYVVLLRRSHGDRSRGKAFSQSFREVAGRFLASEASTHLLVFGMEQRLPPNSELVQAVLRLFQEPVPKAALHRHGGLSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFLGPRQCIASETASLPMIVSIKEGGRTRPQVVSLPNMRVQKCSCASDGALVPRRLQPSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 153 - protein, 593 aa, Artificial Sequence
  CX196
LTGEQLLGSLLRQLQLKEVPTLDRADMEELVIPTHVRAQYVALLQRSHGDRSGGKGFSQSFREVAGRFLASEASTHLLVFGMEQRLPPNSELVQAVLRLFQEPVPKAALHRHGGLSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFLGPRQCIASETASLPMIVSIKEGGRTRPQVVSLPNMRVQKCSCASDGALVPRRLQPSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 154 - protein, 593 aa, Artificial Sequence
  CX197
LTGEQLLGSLLRQLQLKEVPTLDRADMEELVIPTHVRAQYVALLQRSHGDRSGGKGFSQSFREVAGRFLASEASTHLLVFGMEQRLPPNSELVQAVLRLFQEPVPKAALHRHGALSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFLGPRQCIASETASLPMIVSIKEGGRTRPQVVSLPNMRVQKCSCASDGALVPRRLQPSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 155 - protein, 593 aa, Artificial Sequence
  CX201
LTEEQLLGSLLRQLQLSEVPVLDRADMEKLVIPAHVRAQYVALLQRSHGDRSGGKGFSQSFREVAGRFLASEASTHLLVFGMEQRLPPNSELVQAVLRLFQEPVPKAALHRHGGLSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFLGPRQCIASETASLPMIVSIKEGGRTRPQVVSLPNMRVQKCSCASDGALVPRRLQPSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 156 - protein, 593 aa, Artificial Sequence
  CX203
LTEEQLLGSLLRQLQLSEVPVLDRADMEKLVIPAHVRAQYVALLQRSHGDRSGGKGFSQSFREVAGRFLASEASTHLLVFGMEQRLPPNSELVQAVLRLFQEPVPKAALHRHERLSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFLGPRQCIASETASLPMIVSIKEGGRTRPQVVSLPNMRVQKCSCASDGALVPRRLQPSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 157 - protein, 593 aa, Artificial Sequence
  CX206
LTEEQLLGSLLRQLQLSEVPVLDRADMEKLVIPAHVRAQYVALLQRSHGDRSGGKGFSQSFREVAGRFLASEASTHLLVFGMEQRLPPNSELVQAVLRLFQEPVPKAALHRHGGLSPRSAQARVTVEWLRVRDDGSNRTSLIDSRLVSVHESGWKAFDVTEAVNFWQQLSRPRQPLLLQVTVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFLGPRQCIASETASLPMIVSIKEGGRTRPQVVSLPNMRVQKCSCASDGALVPRRLQPSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 158 - protein, 593 aa, Artificial Sequence
  CX207
LTEEQLLGSLLRQLQLSEVPVLDRADMEKLVIPAHVRAQYVALLQRSHGDRSGGKGFSQSFREVAGRFLASEASTHLLVFGMEQRLPPNSELVQAVLRLFQEPVPKAALHRHGGLSPRSAQARVTVEWLRVRDDGSNRTSLIDSRLVSVHESGWKAFDVTEAVNFWQQLSRPRQPLLLQVSVQREHLGPLASGAHKLVRFAGQGAPAGLGEPQLELHTLDLRDYGAQGDCDPEAPMTEGTRCCRQEMYIDLQGMKWAKNWVLEPPGFLAYECVGTCQQPPEALAFNWPFLGPRQCIASETASLPMIVSIKEGGRTRPQVVSLPNMRVQKCSCASDGALVPRRLQPSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 159 - protein, 593 aa, Artificial Sequence
  CX208
LTEEQLLGSLLRQLQLSEVPVLDRADMEKLVIPAHVRAQYVALLQRSHGDRSGGKGFSQSFREVAGRFLASEASTHLLVFGMEQRLPPNSELVQAVLRLFQEPVPKAALHRHGGLSPRSAQARVTVEWLRVRDDGSNRTSLIDSRLVSVHESGWKAFDVTEAVNFWQQLSRPRQPLLLQVTVQREHLGPLASGAHKLVRFAGQGAPAGLGEPQLELHTLDLRDYGAQGDCDPEAPMTEGTRCCRQEMYIDLQGMKWAKNWVLEPPGFLAYECVGTCQQPPEALAFNWPFLGPRQCIASETASLPMIVSIKEGGRTRPQVVSLPNMRVQKCSCASDGALVPRRLQPSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 160 - protein, 593 aa, Artificial Sequence
  CX201(L311D)
LTEEQLLGSLLRQLQLSEVPVLDRADMEKLVIPAHVRAQYVALLQRSHGDRSGGKGFSQSFREVAGRFLASEASTHLLVFGMEQRLPPNSELVQAVLRLFQEPVPKAALHRHGGLSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFDGPRQCIASETASLPMIVSIKEGGRTRPQVVSLPNMRVQKCSCASDGALVPRRLQPSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 161 - protein, 593 aa, Artificial Sequence
  CX201(L311E)
LTEEQLLGSLLRQLQLSEVPVLDRADMEKLVIPAHVRAQYVALLQRSHGDRSGGKGFSQSFREVAGRFLASEASTHLLVFGMEQRLPPNSELVQAVLRLFQEPVPKAALHRHGGLSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFEGPRQCIASETASLPMIVSIKEGGRTRPQVVSLPNMRVQKCSCASDGALVPRRLQPSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 162 - protein, 593 aa, Artificial Sequence
  CX201(P313D)
LTEEQLLGSLLRQLQLSEVPVLDRADMEKLVIPAHVRAQYVALLQRSHGDRSGGKGFSQSFREVAGRFLASEASTHLLVFGMEQRLPPNSELVQAVLRLFQEPVPKAALHRHGGLSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFLGDRQCIASETASLPMIVSIKEGGRTRPQVVSLPNMRVQKCSCASDGALVPRRLQPSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 163 - protein, 593 aa, Artificial Sequence
  CX201(P313E)
LTEEQLLGSLLRQLQLSEVPVLDRADMEKLVIPAHVRAQYVALLQRSHGDRSGGKGFSQSFREVAGRFLASEASTHLLVFGMEQRLPPNSELVQAVLRLFQEPVPKAALHRHGGLSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFLGERQCIASETASLPMIVSIKEGGRTRPQVVSLPNMRVQKCSCASDGALVPRRLQPSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 164 - protein, 593 aa, Artificial Sequence
  CX201(P313S)
LTEEQLLGSLLRQLQLSEVPVLDRADMEKLVIPAHVRAQYVALLQRSHGDRSGGKGFSQSFREVAGRFLASEASTHLLVFGMEQRLPPNSELVQAVLRLFQEPVPKAALHRHGGLSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFLGSRQCIASETASLPMIVSIKEGGRTRPQVVSLPNMRVQKCSCASDGALVPRRLQPSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 165 - protein, 593 aa, Artificial Sequence
  CX201(R314K)
LTEEQLLGSLLRQLQLSEVPVLDRADMEKLVIPAHVRAQYVALLQRSHGDRSGGKGFSQSFREVAGRFLASEASTHLLVFGMEQRLPPNSELVQAVLRLFQEPVPKAALHRHGGLSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFLGPKQCIASETASLPMIVSIKEGGRTRPQVVSLPNMRVQKCSCASDGALVPRRLQPSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 166 - protein, 593 aa, Artificial Sequence
  CX201(R314Q)
LTEEQLLGSLLRQLQLSEVPVLDRADMEKLVIPAHVRAQYVALLQRSHGDRSGGKGFSQSFREVAGRFLASEASTHLLVFGMEQRLPPNSELVQAVLRLFQEPVPKAALHRHGGLSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFLGPQQCIASETASLPMIVSIKEGGRTRPQVVSLPNMRVQKCSCASDGALVPRRLQPSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 167 - protein, 593 aa, Artificial Sequence
  CX201(L359D)
LTEEQLLGSLLRQLQLSEVPVLDRADMEKLVIPAHVRAQYVALLQRSHGDRSGGKGFSQSFREVAGRFLASEASTHLLVFGMEQRLPPNSELVQAVLRLFQEPVPKAALHRHGGLSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFLGPRQCIASETASLPMIVSIKEGGRTRPQVVSLPNMRVQKCSCASDGADVPRRLQPSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 168 - protein, 593 aa, Artificial Sequence
  CX201(L359E)
LTEEQLLGSLLRQLQLSEVPVLDRADMEKLVIPAHVRAQYVALLQRSHGDRSGGKGFSQSFREVAGRFLASEASTHLLVFGMEQRLPPNSELVQAVLRLFQEPVPKAALHRHGGLSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFLGPRQCIASETASLPMIVSIKEGGRTRPQVVSLPNMRVQKCSCASDGAEVPRRLQPSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 169 - protein, 593 aa, Artificial Sequence
  CX201(P361D)
LTEEQLLGSLLRQLQLSEVPVLDRADMEKLVIPAHVRAQYVALLQRSHGDRSGGKGFSQSFREVAGRFLASEASTHLLVFGMEQRLPPNSELVQAVLRLFQEPVPKAALHRHGGLSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFLGPRQCIASETASLPMIVSIKEGGRTRPQVVSLPNMRVQKCSCASDGALVDRRLQPSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 170 - protein, 593 aa, Artificial Sequence
  CX201(P361E)
LTEEQLLGSLLRQLQLSEVPVLDRADMEKLVIPAHVRAQYVALLQRSHGDRSGGKGFSQSFREVAGRFLASEASTHLLVFGMEQRLPPNSELVQAVLRLFQEPVPKAALHRHGGLSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFLGPRQCIASETASLPMIVSIKEGGRTRPQVVSLPNMRVQKCSCASDGALVERRLQPSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 171 - protein, 593 aa, Artificial Sequence
  CX201(P361S)
LTEEQLLGSLLRQLQLSEVPVLDRADMEKLVIPAHVRAQYVALLQRSHGDRSGGKGFSQSFREVAGRFLASEASTHLLVFGMEQRLPPNSELVQAVLRLFQEPVPKAALHRHGGLSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFLGPRQCIASETASLPMIVSIKEGGRTRPQVVSLPNMRVQKCSCASDGALVSRRLQPSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 172 - protein, 593 aa, Artificial Sequence
  CX201(R362K)
LTEEQLLGSLLRQLQLSEVPVLDRADMEKLVIPAHVRAQYVALLQRSHGDRSGGKGFSQSFREVAGRFLASEASTHLLVFGMEQRLPPNSELVQAVLRLFQEPVPKAALHRHGGLSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFLGPRQCIASETASLPMIVSIKEGGRTRPQVVSLPNMRVQKCSCASDGALVPKRLQPSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 173 - protein, 593 aa, Artificial Sequence
  CX201(R362Q)
LTEEQLLGSLLRQLQLSEVPVLDRADMEKLVIPAHVRAQYVALLQRSHGDRSGGKGFSQSFREVAGRFLASEASTHLLVFGMEQRLPPNSELVQAVLRLFQEPVPKAALHRHGGLSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFLGPRQCIASETASLPMIVSIKEGGRTRPQVVSLPNMRVQKCSCASDGALVPQRLQPSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 174 - protein, 593 aa, Artificial Sequence
  CX201(L311D/R362Q)
LTEEQLLGSLLRQLQLSEVPVLDRADMEKLVIPAHVRAQYVALLQRSHGDRSGGKGFSQSFREVAGRFLASEASTHLLVFGMEQRLPPNSELVQAVLRLFQEPVPKAALHRHGGLSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFDGPRQCIASETASLPMIVSIKEGGRTRPQVVSLPNMRVQKCSCASDGALVPQRLQPSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 175 - protein, 593 aa, Artificial Sequence
  CX201(L311E/R362Q)
LTEEQLLGSLLRQLQLSEVPVLDRADMEKLVIPAHVRAQYVALLQRSHGDRSGGKGFSQSFREVAGRFLASEASTHLLVFGMEQRLPPNSELVQAVLRLFQEPVPKAALHRHGGLSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFEGPRQCIASETASLPMIVSIKEGGRTRPQVVSLPNMRVQKCSCASDGALVPQRLQPSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 176 - protein, 593 aa, Artificial Sequence
  CX201(L311D/P361D/R362Q)
LTEEQLLGSLLRQLQLSEVPVLDRADMEKLVIPAHVRAQYVALLQRSHGDRSGGKGFSQSFREVAGRFLASEASTHLLVFGMEQRLPPNSELVQAVLRLFQEPVPKAALHRHGGLSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFDGPRQCIASETASLPMIVSIKEGGRTRPQVVSLPNMRVQKCSCASDGALVDQRLQPSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 177 - protein, 593 aa, Artificial Sequence
  CX201(L311E/P361D/R362Q)
LTEEQLLGSLLRQLQLSEVPVLDRADMEKLVIPAHVRAQYVALLQRSHGDRSGGKGFSQSFREVAGRFLASEASTHLLVFGMEQRLPPNSELVQAVLRLFQEPVPKAALHRHGGLSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFEGPRQCIASETASLPMIVSIKEGGRTRPQVVSLPNMRVQKCSCASDGALVDQRLQPSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 178 - protein, 593 aa, Artificial Sequence
  CX201s
LTEEQLLGSLLRQLQLSEVPVLDRADMEKLVIPAHVRAQYVALLQRSHGDRSGGKGFSQSFREVAGRFLASEASTHLLVFGMEQRLPPNSELVQAVLRLFQEPVPKAALHRHGGLSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFLGPRQCIASETASLPMIVSIKEGGRTRPQVVSLPNMRVQKCSCASDGALVPRRLQPSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2022/0380424 A1
(43) Pub. Date: Dec. 1, 2022

(54) FUSION PROTEIN COMPRISING HUMAN LEFTY A PROTEIN VARIANTS AND USE THEREOF

(71) Applicants: CHONG KUN DANG PHARMACEUTICAL CORP., Seoul (KR); SAMSUNG LIFE PUBLIC WELFARE FOUNDATION, Seoul (KR)

(72) Inventors: Sun-Young JEONG, Gyeonggi-do (KR); Kyoung Woo LEE, Gyeonggi-do (KR); Seung Kee MOON, Gyeonggi-do (KR); Sung Jun KANG, Gyeonggi-do (KR); Byung-OK CHOI, Seoul (KR); Geon KWAK, Gyeonggi-do (KR); Jong Wook CHANG, Seoul (KR); Jong Hyun KIM, Gyeonggi-do (KR)

(21) Appl. No.: 17/416,484

(22) PCT Filed: Dec. 17, 2019
(86) PCT No.: PCT/KR2019/017917

§ 371 Date: Jun. 19, 2021

(30) Foreign Application Priority Data
Dec. 20, 2018 (KR) 10-2018-0166802

(51) Int. Cl. C07K 14/495; A61P 21/00

(52) U.S. Cl. CPC C07K 14/495; A61P 21/00; C07K 2319/02; C07K 2319/30; C07K 2319/31; A61K 38/00

(57) ABSTRACT

The present invention relates to a human Lefty A protein variant with improved productivity and stability, a fusion protein comprising the protein variant, and a composition for preventing and/or treating neuromuscular disease comprising the protein variant or the fusion protein. According to the present invention, a human Lefty A protein variant and a fusion protein comprising the variant are constructed, which have better stability than naturally occurring human Lefty A protein, and thus are expressed at high levels and produced in high yield in animal cells. In addition, administration of the constructed human Lefty A protein variant or fusion protein can restore the nerve and motor functions of nerve disease model animals. Accordingly, the use of the human Lefty A protein variant or fusion protein can effectively prevent or treat various nerve diseases and muscle diseases.

### TECHNICAL FIELD

The present invention relates to a human Lefty A protein variant with improved productivity and stability, a fusion protein comprising the protein variant, and a composition for preventing and/or treating neuromuscular disease, comprising the protein variant or the fusion protein.

### BACKGROUND ART

Lefty (left-right determination factor) is a morphological differentiation factor that belongs to the transforming growth factor-beta (TGF-β) superfamily and plays a crucial role in embryonic cell differentiation and development by binding to other TGF-β ligands (Shiratori H et al., Semin Cell Dev Biol. 2014; 32:80-4).

Human LEFTY genes include two genes (Lefty A protein-encoding LEFTY2 and Lefty B protein-encoding LEFTY1) creased by independent duplication (Gharib W H, Robinson-Rechavi M, Brief Bioinform. 2011; 12(5):436-41). The deduced amino acid sequences of Lefty A and Lefty B shows 96% identity to each other.

Human LEFTY gene is translated into a polypeptide consisting of 366 amino acids, and the N-terminal signal peptide consisting of 21 amino acids is cleaved during translocation into the endoplasmic reticulum (ER) and finally extracellularly released as a 42 kDa pro-protein. The released 42 kDa Lefty protein can be processed by removal of the propeptide through protease cleavage at its N-terminal cleavage site (RXXR). Unlike other members of TGF-b family, Lefty has two, rather than one, putative cleavage sites (RXXRs) and does not have the conserved cysteine necessary for homodimer formation.(Juan et al., Genes to Cells. 2001; 6:923-30).

Lefty polypeptide may be cleaved at the carboxyl end of the second arginine of each cleavage site by proprotein convertases and processed into 34 kDa and 28 kDa polypeptides. The proteolytic processing of the Lefty polypeptide could be prevented by mutation of both arginine residues at the first cleavage site(RGKR) to glycine or the first arginine at the second cleavage site(RHGR) to glycine (Ulloa L et al., J Biol Chem. 2001; 276:217387-96).

It was reported that 42 kDa Lefty proprotein, as well as 28 kDa mature protein, can induce MAPK activation (Ulloa L et al., J Biol Chem. 2001; 276:217387-96).

Lefty acts as an endogenous inhibitor of Nodal, which plays an important role in early embryonic development in vertebrate development, such as inducing mesoderm and endoderm differentiation and controlling left-right asymmetry (Schier A F, Annu Rev Cell Biol. 2003; 19:589-621). The exact mechanism of action by which Lefty inhibits Nodal is still unclear, but it is known that Lefty can bind to Nodal and thereby prevent Nodal from binding to Activin receptors (e.g. ActRI or ActRII) and the co-receptor EGF-CFC (Branford et al., Current Biol. 2004; 14:341-3 and Chen et al. Current Biol. 2004; 14:618-24). In addition, it was reported that Lefty inhibits TGF-β or BMP signaling (Ulloa L et al., J Biol Chem. 2001; 276(24): 21397-404).

There are a number of TGF-β superfamily ligands such as BMPs, GDFs, Nodal, Activin and TGF-β s. They play important roles in regulating embryonic development and the function of many cells and organs after birth. For example, TGF-β family members are involved in neural differentiation, and regulate various parts of the central nervous system throughout all stages of development, from early embryos to adult (Myers E A and Kessler J A, Cold Spring Harb Perspect Biol. 2017; 9(8)). TGF-β inhibits the survival of Schwann cells during neural differentiation (Parkinson et al., J Neurosci. 2001; 21:8572-85). BMP7 inhibits Schwann cell myelination process by activating p38 in the peripheral nerve (Liu X et al., Sci Rep. 2016; 6:31049). GDF8(Myostatin) inhibits skeletal muscle growth and its overexpression induces skeletal muscle atrophy(Elkina Y et al., J Cachexia Sarcopenia Muscle. 2001; 2:143-51).

Charcot-Marie-Tooth disease (CMT) is one of the most common inherited peripheral neuropathy with a prevalence of 1 in 2,500. More than 80 genes causing CMT have been identified. CMT patients develop slowly progressive muscular atrophy and sensory loss that starts in the lower limbs.

CMT is a phenotypically and genetically heterogeneous disease and divided into demyelinating type (CMT1) and axonal type (CMT2) on the basis of upper limb motor nerve conduction velocities (MCVs). Among these types, the demyelinating type (CMT1) accounts for about 70% of all the CMT patients and is caused by overexpression or mutation of myelin proteins such as PMP22 and MPZ. Although the pathological mechanisms have not been elucidated, it has been suggested that ER stress is induced by overexpression of or mutations in proteins, with the subsequent apoptosis of Schwann cells, as one of the mechanisms. Since no drugs are approved for treatment of CMT, there is a growing interest in developing effective therapeutics for CMT.

Under this technical background, the present inventors have constructed a variant with increased productivity and stability by introducing a mutation into human Lefty A protein so as to allow the Lefty A protein to be solubilized for the purpose of treating neuropathy or muscle disease, thereby completing the present invention.

The information disclosed in the Background Art section is only for the enhancement of understanding of the background of the present invention, and therefore may not contain information that forms a prior art that would already be known to a person of ordinary skill in the art.

### DISCLOSURE OF INVENTION

### Technical Problem

It is an object of the present invention to provide a human Lefty A protein variant with increased productivity and stability.

Another object of the present invention is to provide a fusion protein comprising the human Lefty A protein variant.

Still another object of the present invention is to provide a nucleic acid molecule encoding the fusion protein, an expression vector comprising the nucleic acid molecule, a recombinant cell into which the expression vector has been introduced, and a method of producing a fusion protein using the recombinant cell.

Yet another object of the present invention is to provide a composition for preventing and/or treating neuromuscular disease, which comprises either the protein variant or the fusion protein.

A further object of the present invention is to provide a method for preventing and/or treating neuromuscular disease, which comprises administering either the protein variant or the fusion protein to a subject.

A still further object of the present invention is to provide a use of either the protein variant or the fusion protein, for the prevention and/or treatment of neuromuscular disease.

A yet further object of the present invention is to provide a use of either the protein variant or the fusion protein, for the manufacture of a medicament for treating neuromuscular disease.

### Technical Solution

To achieve the above objects, the present invention provides a human Lefty A protein variant comprising the amino acid sequence of L22 to P366 of a human Lefty A protein having the amino acid sequence of SEQ ID NO: 131, wherein the human Lefty A protein variant comprising: (1) a substitution of one or more amino acid residues at processing sites (R74 to R77 and R132 to R135); and (2) a substitution of one or more amino acid residues in a propeptide domain (L22 to S73).

The present invention also provides a fusion protein comprising the human Lefty A protein variant.

The present invention also provides a nucleic acid molecule encoding the fusion protein comprising a human Lefty A protein variant, an expression vector comprising the nucleic acid molecule, a recombinant cell which the expression vector has been introduced, and a method of producing a fusion protein comprising a human Lefty A protein variant using the recombinant cell.

The present invention also provides a composition for preventing and/or treating neuromuscular disease, which comprises either the human Lefty A protein variant or the fusion protein comprising the human Lefty A protein variant.

The present invention also provides a method for preventing and/or treating neuromuscular disease, which comprises administering either the human Lefty A protein variant or the fusion protein comprising the human Lefty A protein variant to a subject.

The present invention also provides a use of either the human Lefty A protein variant or the fusion protein comprising the human Lefty A protein variant, for the prevention and/or treatment of neuromuscular disease.

The present invention also provides a use of either the human Lefty A protein variant or the fusion protein comprising the human Lefty A protein variant, for the manufacture of a medicament for treating neuromuscular disease.

### BRIEF DESCRIPTION OF THE DRAWINGS

FIG. 1 is schematic diagram of human Lefty A fusion proteins.

FIG. 2 shows Western blot analysis of culture supernatants to evaluate the expression of HSA-fused human Lefty A protein variants.

FIG. 3 shows SDS-PAGE analysis of 42 LFc protein after ProA Affinity purification.

FIG. 4 shows SDS-PAGE and SEC analysis of Lefty A protein variants (combination) after ProA Affinity purification.

FIG. 5 shows cell viability assay and Western blot analysis with anti-PARP antibody to evaluate effects of co-culture with human Wharton's jelly-derived human mesenchymal stem cells (MSCs) on S16 schwann cell apoptosis induced by thapsigargin.

FIG. 6 shows electrophysiological assessment of therapeutic effects of human Lefty A fusion protein variants in Tr-J mice.

FIG. 7 shows Western blot analysis to assess expression of KROX20 and MBP (A) or Krox20 and phosphorylated/unphosphorylated p38 (B) in RT4 Schwann cells treated with nodal or/and human Lefty A fusion protein variant.

FIG. 8 shows electrophysiological assessment of therapeutic effects of intraperitoneal injection of human Lefty A fusion protein variant in C22 mice.

FIG. 9 shows rotarod (A) and hindlimb grip Strength (B) tests in C22 mice intraperitoneally injected with human Lefty A fusion protein variant.

FIG. 10 shows magnetic resonance imaging analysis of the gastrocnemius muscle of C22 mice intraperitoneally injected with human Lefty A fusion protein variant.

FIG. 11 shows gait analysis of wild-type mice and C22 mice injected intraperitoneally with human Lefty A fusion protein variant.

FIG. 12 shows electrophysiological assessment of therapeutic effects of intraperitoneal injection of human Lefty A fusion protein variant in C22 mice.

FIG. 13 shows hindlimb grip strength (A) and rotarod (B) tests in C22 mice intraperitoneally injected with human Lefty A fusion protein variant.

FIG. 14 shows gait analysis of C22 mice intraperitoneally injected with human Lefty A fusion protein variant.

FIG. 15 shows electrophysiological analysis in C22 mice injected subcutaneously with human Lefty A fusion protein variant for 4 weeks starting at 3 weeks of age.

FIG. 16 shows hindlimb grip strength test in C22 mice injected subcutaneously with human Lefty A fusion protein variant for 4 weeks starting at 3 weeks of age.

FIG. 17 shows A204 cell-based luciferase reporter assay to evaluate to effect of human Lefty A fusion protein variant on signaling by myostatin.

FIG. 18 shows Western blot analysis of sciatic nerve lysates of C22 mice injected with human Lefty A fusion protein variant.

FIG. 19 shows electrophysiological analysis in C22 mice injected subcutaneously with human Lefty A fusion protein variant for 4 weeks starting at 5 weeks of age. 3

FIG. 20 shows rotarod (A) and whole-limb grip strength (B) tests in C22 mice injected subcutaneously with human Lefty A fusion protein variant for 4 weeks starting at 5 weeks of age.

FIG. 21 shows Western blot analysis to evaluate effect of human Lefty A fusion protein variant on Nodal signaling in P19 cells.

FIGS. 22 and 23 show the amino acid sequences of the human Lefty A fusion protein variants according to the present invention.

### BEST MODE FOR CARRYING OUT THE INVENTION

Unless defined otherwise, all the technical and scientific terms used herein have the same meaning as those generally understood by one of ordinary skill in the art to which the invention pertains. Generally, the nomenclature used herein and the experiment methods, which will be described below, are those well known and commonly employed in the art.

In the present invention, a fusion protein comprising a human Lefty A (Uniprot No. 000292; NCBI DB NM 003240) protein variant with increased productivity and stability has improved productivity and stability in CHO cells compared to wild-type protein and previously reported human Lefty A variants, and also binds to human Nodal and inhibits Nodal-mediated signaling, which can contribute to improving symptoms of peripheral neuropathy. Thus, the fusion protein may be effectively used alone or in combination with a conventional pharmaceutically acceptable carrier, neuropathy therapeutic agent, muscle disease therapeutic agent, etc., as a composition for preventing or treating neuropathy and muscle disease.

Therefore, in one aspect, the present invention is directed to a human Lefty A protein variant comprising the amino acid sequence of L22 to P366 of a human Lefty A protein having the amino acid sequence of SEQ ID NO: 131, wherein the human Lefty A protein variant comprising: (1) a substitution of one or more amino acid residues at processing sites (R74 to R77 and R132 to R135); and (2) a substitution of one or more amino acid residues in a propeptide domain (L22 to S73).

M1 to A21 in the human Lefty A protein having the amino acid sequence of SEQ ID NO: 131 correspond to a signal peptide.

The human Lefty A protein variant according to the present invention is meant also to include variants in which amino acid residues at specific amino acid residue positions are conservatively substituted.

As used herein, the term “conservative substitution” refers to modifications of a Lefty A protein variant that involve the substitution of one or more amino acids with amino acids having similar biochemical properties that do not result in loss of the biological or biochemical function of the Lefty A protein variant.

A “conservative amino acid substitution” is one in which the amino acid residue is replaced with an amino acid residue having a similar side chain. Families of amino acid residues having similar side chains have been defined and are well known in the art to which the present invention pertains. These families include amino acids (e.g., lysine, arginine and histidine) with basic side chains, amino acids (e.g., aspartic acid and glutamic acid) with acidic side chains, amino acids (e.g., glycine, aspargin, glutamine, serine, threonine, tyrosine, and cysteine) with uncharged polar side chains, amino acids (e.g., alanine, valine, leucine, isoleucine, proline, phenylalanine, methionine, and tryptophan) with nonpolar side chains, amino acids (e.g., threonine, valine, and isoleucine) with beta-branched side chains, and amino acids (e.g., tyrosine, phenylalanine, tryptophan, and histidine) with aromatic side chains.

It is envisioned that the Lefty A protein variant of the present invention may still retain activity although it has conservative amino acid substitutions.

In addition, the human Lefty A protein variant according to the present invention is interpreted to include a Lefty A protein variant having substantially the same function and/or effect with those/that of the Lefty A protein variant according to the present invention, and having an amino acid sequence homology of at least 80% or 85%, preferably at least 90%, more preferably at least 95%, most preferably at least 99% to the Lefty A protein variant according to the present invention.

Preferably, the human Lefty A protein variant according to the present invention may comprise the amino acid sequence of L22 to P366 of any one selected from the group consisting of SEQ ID NO: 86 to SEQ ID NO: 111, or the amino acid sequence of L23 to P367 of any one selected from the group of consisting SEQ ID NOS: 112 to 129 and 133, but is not limited thereto.

It is to be understood that the Lefty A protein variant according to the present invention also includes those having substantially the same effect as that of the Lefty A protein variant according to the present invention, even though some amino acid residues in the N-terminus, C-terminus or internal amino acid sequence thereof are cleaved or substituted.

In wild-type Lefty A, there is no mutation, that is, no substitution of amino acid residues, in positions R74 to R77, and thus when protease-induced cleavage of the corresponding region occurs, a 34-kDa Lefty A protein fragment is obtained. In addition, there is no mutation in positions R132 to R135, and thus when protease-induced cleavage of the corresponding region occurs, a 28-kDa Lefty A protein fragment is obtained.

Accordingly, the human Lefty A protein variant in the present invention preferably comprises a substitution of one or more amino acids at the processing sites consisting of R74 to R77 and R132 to R135.

In the present invention, the substitution of amino acid residues in the propeptide domain (L22 to S73) may be a substitution of amino acid residues at one or more positions selected from the group consisting of E24, L27, R33, S38, V40, V42, R45, M48, K50, A55, V63, R66, R67, G70 and D71, but is not limited thereto.

Preferably, the substitution of amino acid residues in the propeptide domain (L22 to S73) may be one or more amino acid residue substitutions selected from the group consisting of E24G, S38K, V42T, K50E, A55T, V63A and R66Q.

Most preferably, the substitution of amino acid residues in the propeptide domain (L22 to S73) may comprise V63A, and may further comprise one or more amino acid residue substitutions selected from the group consisting of E24G, S38K, V42T, K50E, A55T and R66Q.

In the present invention, the substitution of one or more amino acid residues at the processing sites (R74 to R77 and R132 to R135) may be one or more amino acid residue substitutions selected from the group consisting of R74G, R77G, R77V, R132G and R135G.

The amino acid sequence of positions R74 to R77 may be RGKR, GGKG, RGKA, RGKV or RHGG, and the amino acid sequence of positions R132 to R135 may be RHGR, GHGR, RHGG, RHER, GHGG, RHGA or RHGV which result from the substitution of one or more amino acid residues at the processing sites (R74 to R77 and R132 to R135).

In the present invention, the human Lefty A protein variant may further comprise a substitution of one or more amino acid residues at a thrombin cleavage site (L311, P313, R314, L359, P361 or R362), but is not limited thereto.

Preferably, the amino acid residues at one or more positions selected from the group consisting of the thrombin cleavage sites L311, P313, R314, L359, P361 and R362 may be substituted with amino acid residues selected from the group consisting of aspartic acid (D), glutamic acid (E), serine (S), lysine (K) and glutamine (Q).

In the present invention, the human Lefty A protein variant may further comprise a substitution of one or more amino acid residues at a fragmentation site (S202 or 5223) with amino acid residues other than serine (S) and cysteine (C).

In the present invention, the human Lefty A protein variant may further comprise a signal peptide at the N-terminus.

In the present specification, the signal peptide is used in the same sense as a signal sequence, and is a short amino acid sequence playing an important role in allowing a new expressed polypeptide to target the endoplasmic reticulum and enter the secretory pathway. When the protein is synthesized in a cell and goes out of the cell, the signal sequence is cleaved and the N-terminus of the Lefty A protein variant begins from L22. The signal sequence that is used in the present invention may be a signal sequence (M1 to A21) derived from Lefty A protein, but is not limited thereto. A signal sequence derived from a protein other than the Lefty A protein may also be used, which may be, for example, an antibody-derived sequence such as MDMRVPAQLLGLLLLWFPGSRC (UniProt: A0A0C4DH73; SEQ ID NO: 132), but is not limited thereto.

In another aspect, the present invention is directed to a fusion protein comprising the human Lefty A protein variant.

In the present invention, the “fusion protein comprising the Lefty A protein variant” is used in the same sense as the “Lefty A fusion protein variant”.

In the present invention, the fusion protein may be produced by fusing the human Lefty A protein variant with Fc or albumin, but is not limited thereto.

Preferably, the fusion protein according to the present invention may be produced by fusing Fc or albumin to the N-terminus or the C-terminus, preferably, the C-terminus of the human Lefty A protein variant. In addition, the fusion protein according to the present invention may be produced by fusing the human Lefty A protein variant with Fc or albumin via a linker.

The Fc in the present invention refers to the Fc (fragment crystallizable) region of an antibody, and may be the Fc of an antibody selected from the group consisting of IgG, IgA, IgM and IgE. Preferably, it may be the Fc of an antibody selected from the group consisting of human IgG, IgA, IgM and IgE, but is not limited thereto.

More preferably, the Fc may be one selected from among human IgG1, IgG2, IgG3 and IgG4, which are derived from human IgG, and most preferably it may be a human IgG1-derived Fc, but not limited thereto. In addition, the Fc in the present invention may be a wild type Fc or an amino acid sequence variant thereof.

In addition, the albumin in the present invention is meant to include all animal-derived albumins. Preferably, a human albumin may be used, but the scope of the present is not limited thereto.

In the present invention, the fusion protein may have any one amino acid sequence selected from the group consisting of amino acid sequences set forth in SEQ ID NOS: 134 to 178, but is not limited thereto.

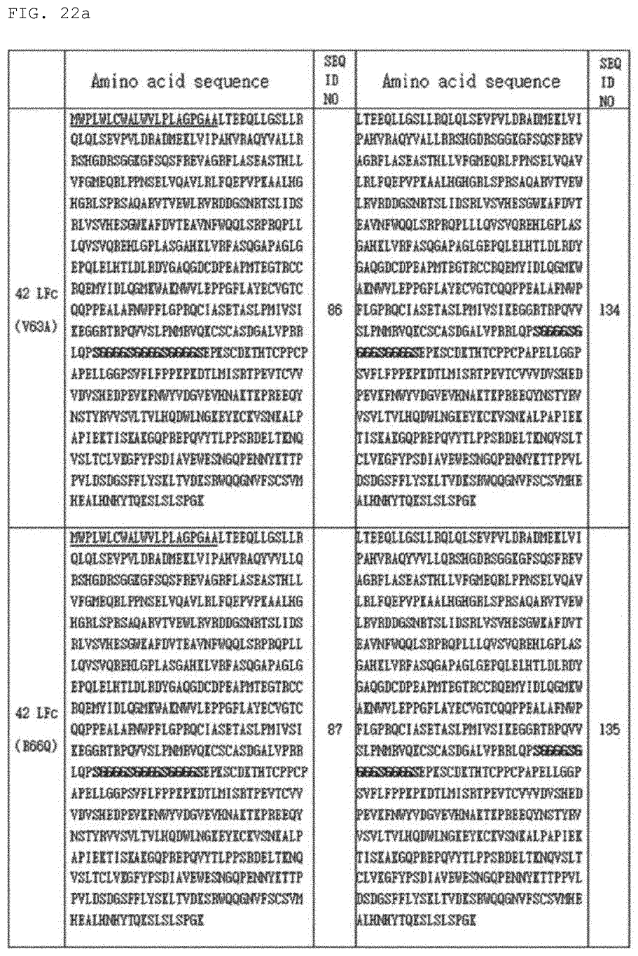

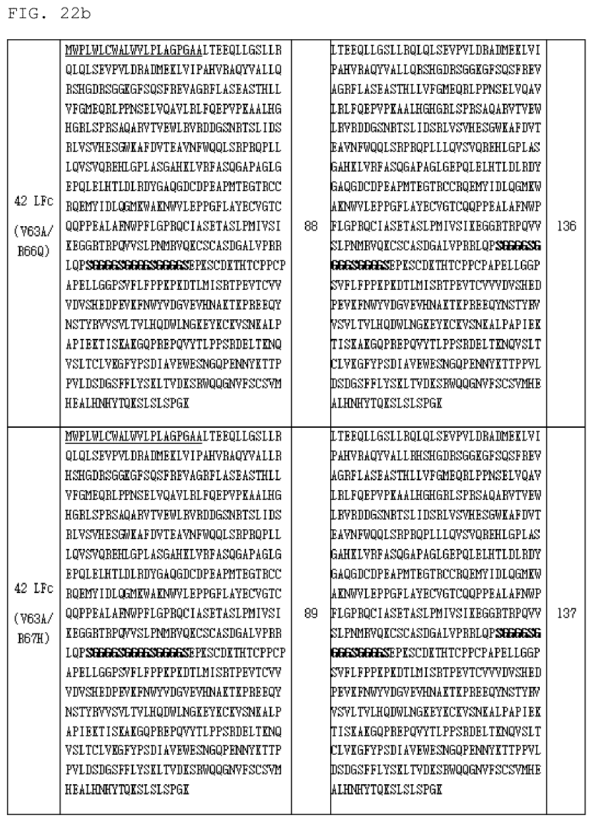

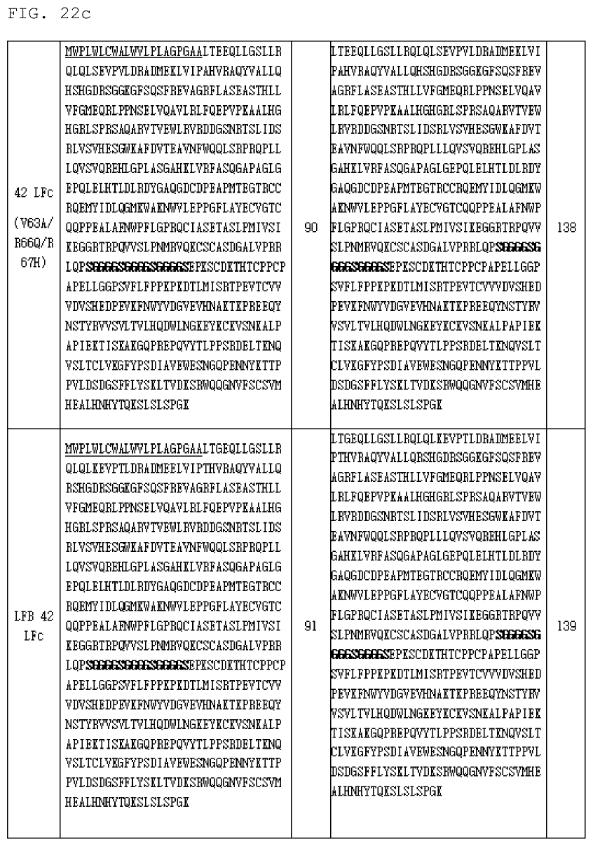

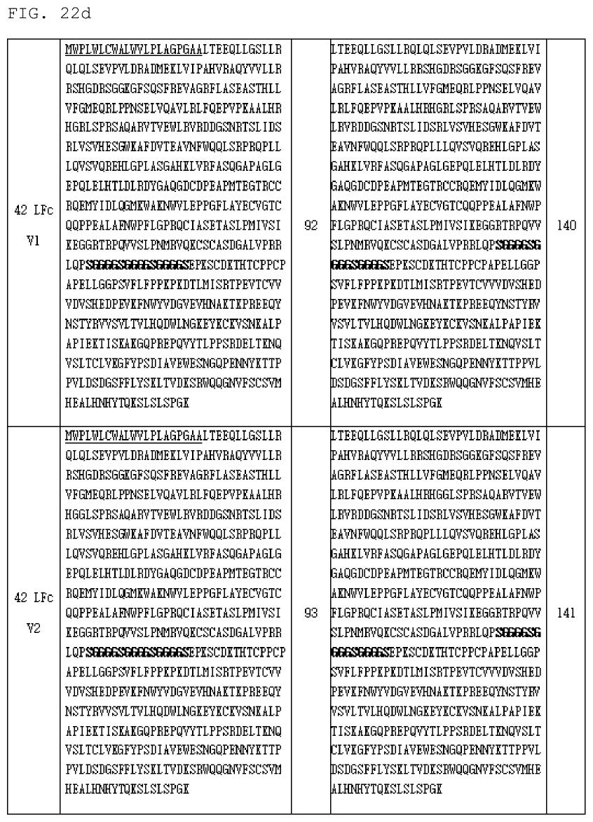

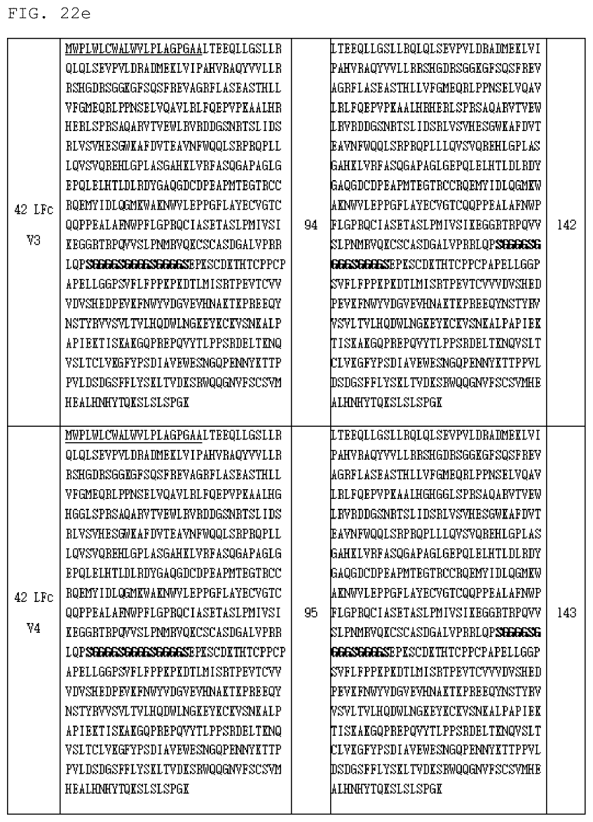

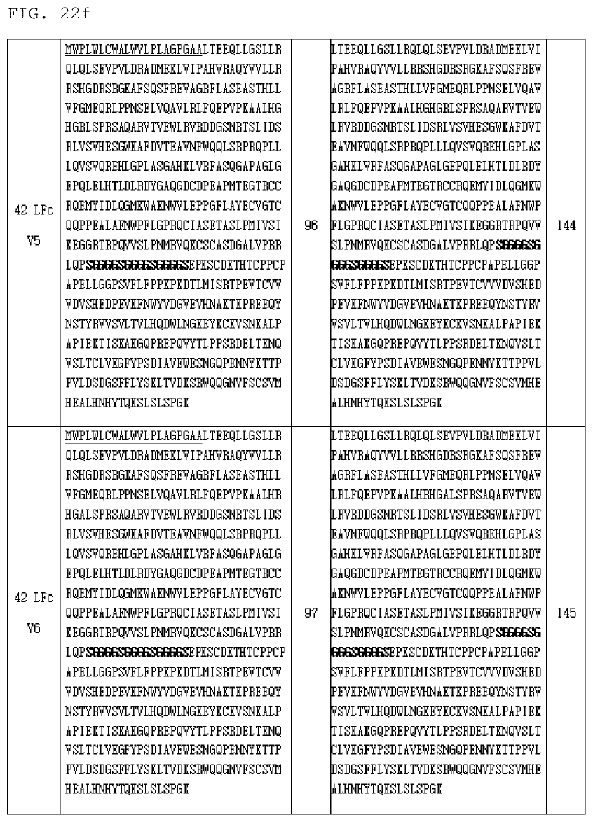

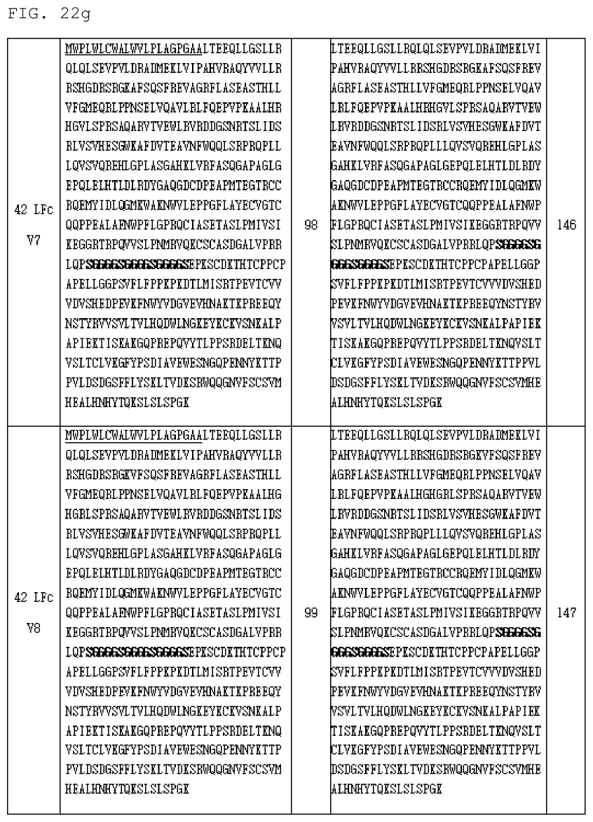

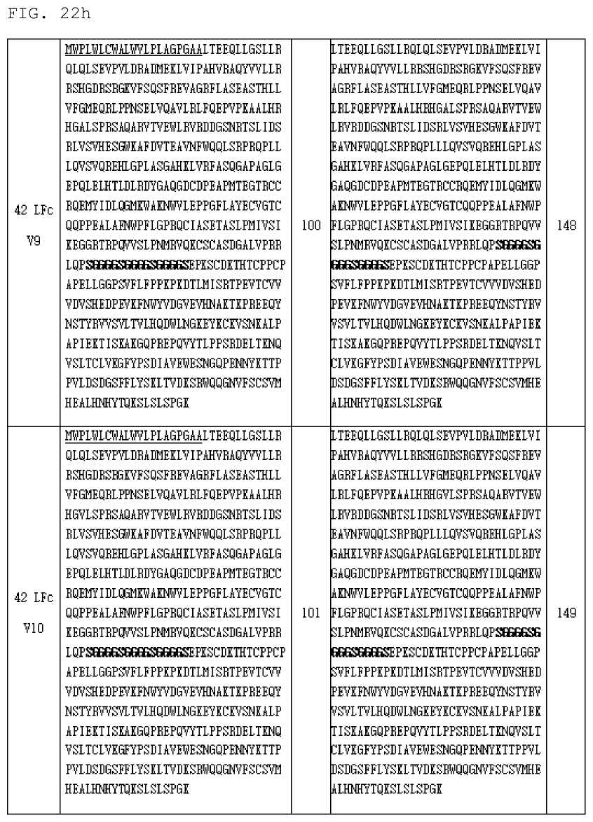

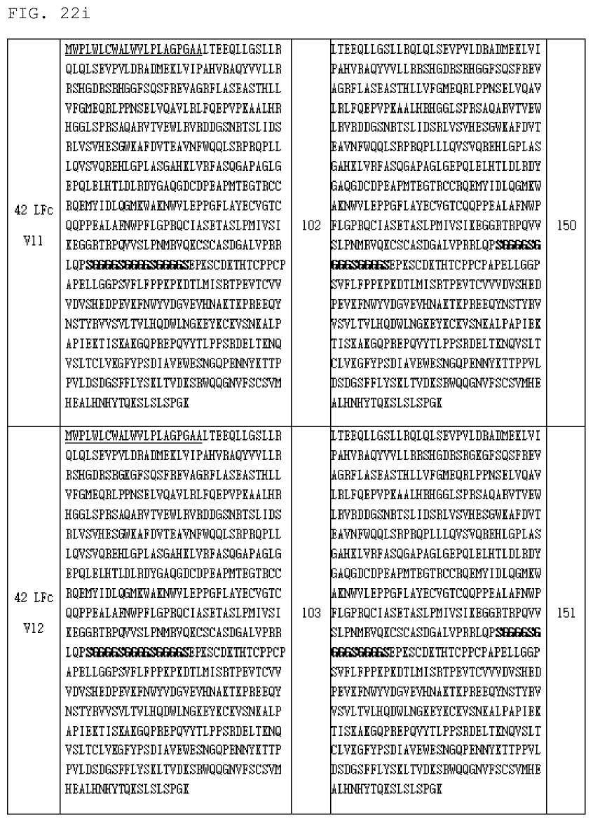

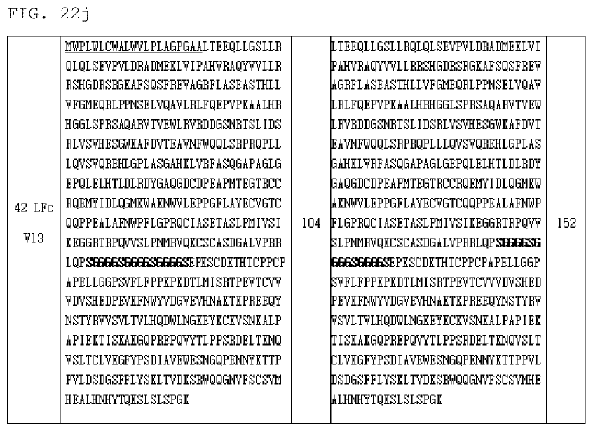

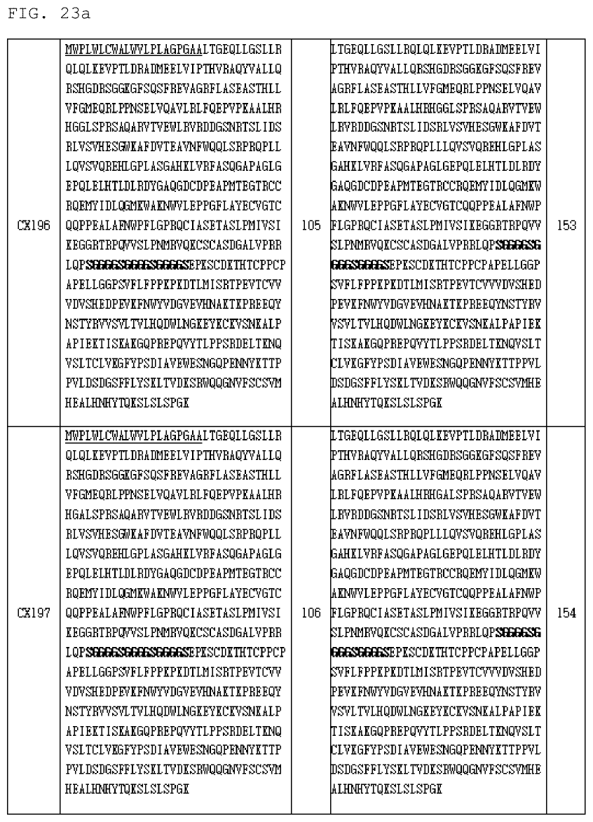

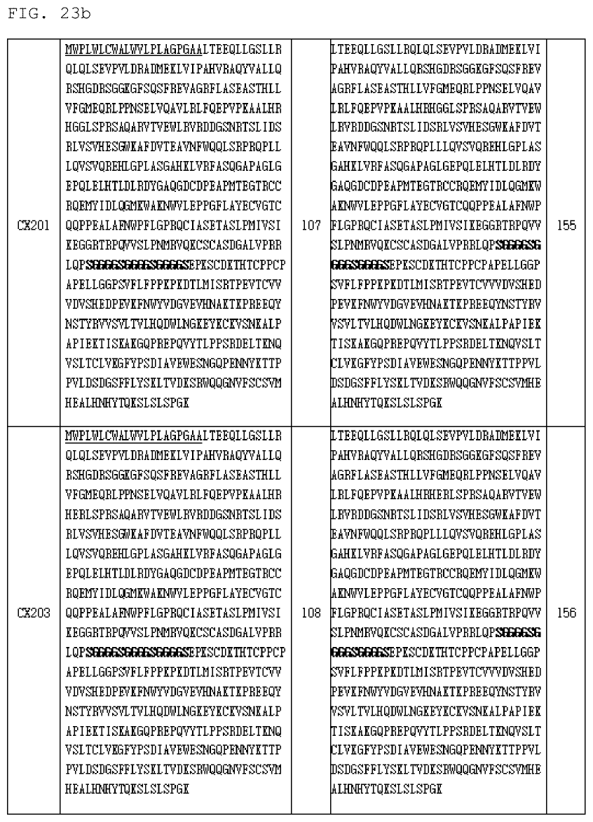

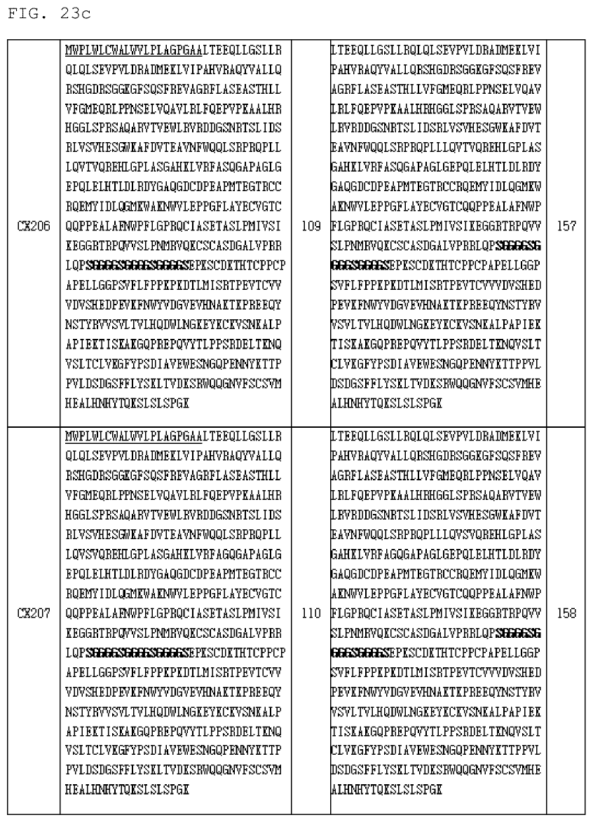

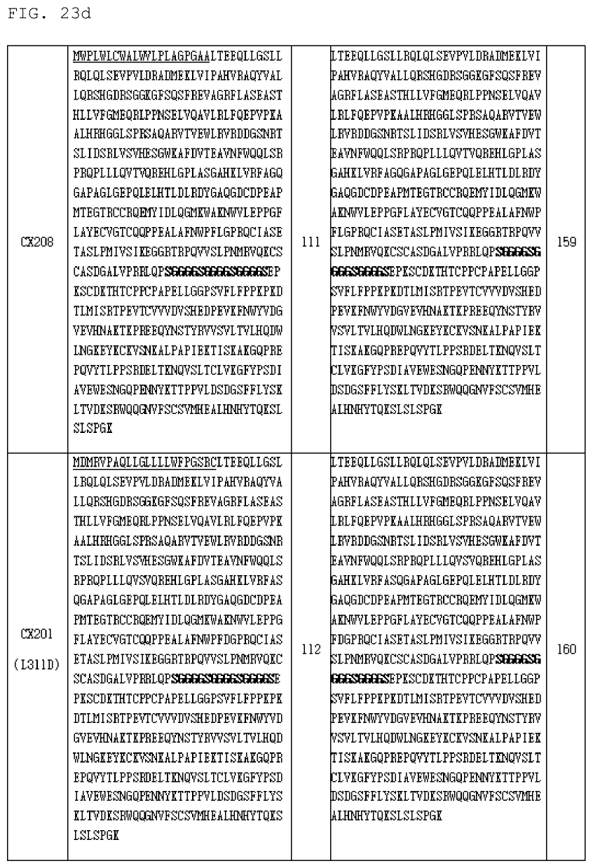

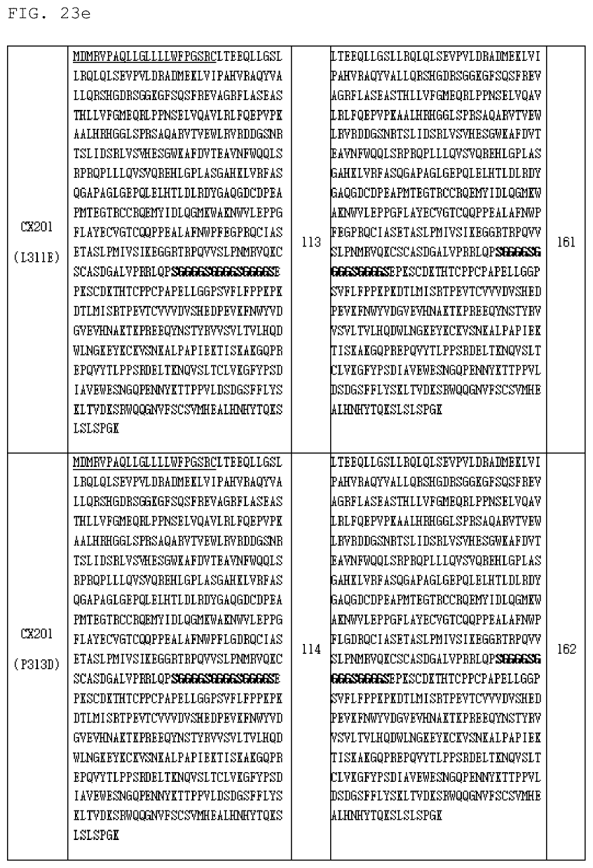

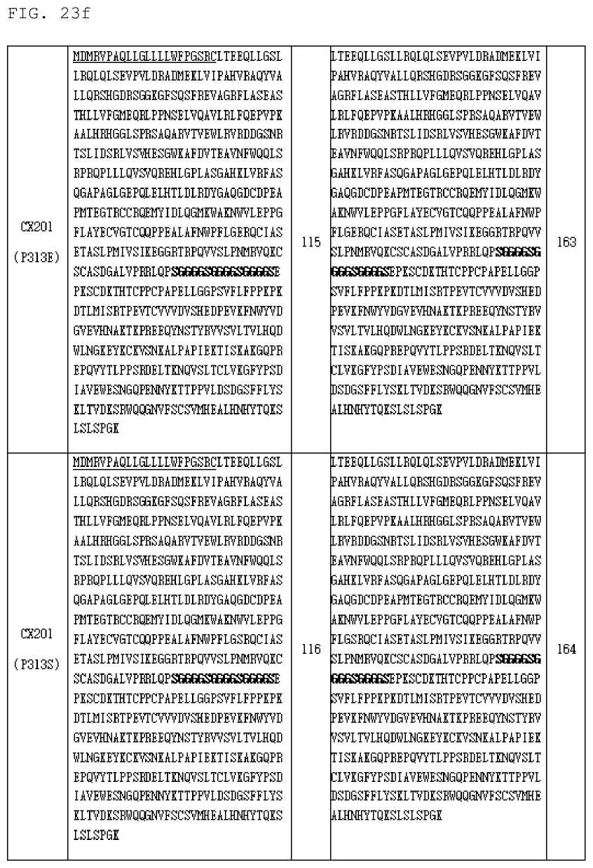

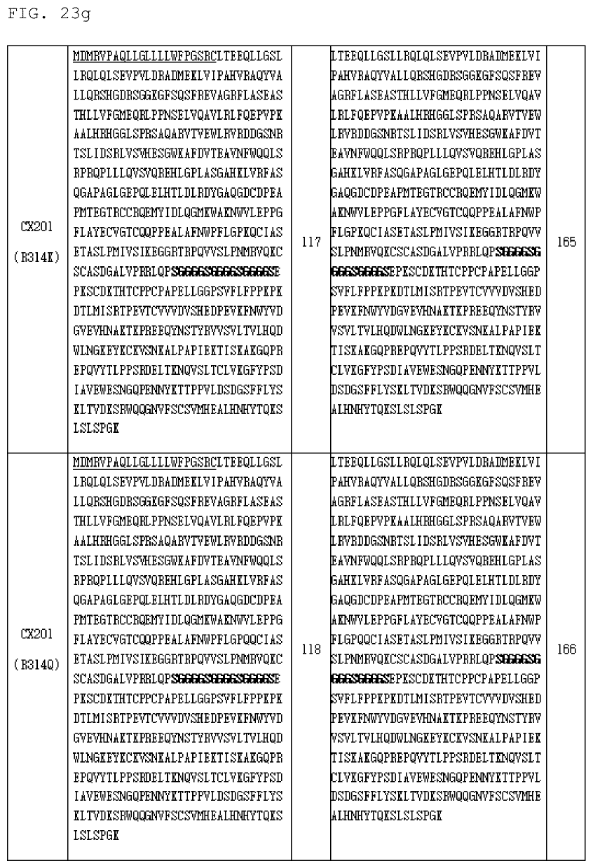

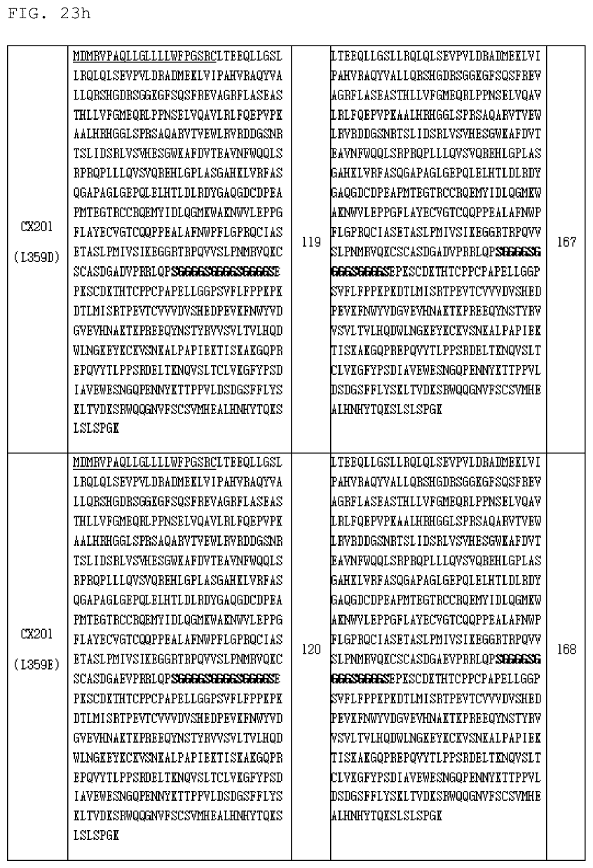

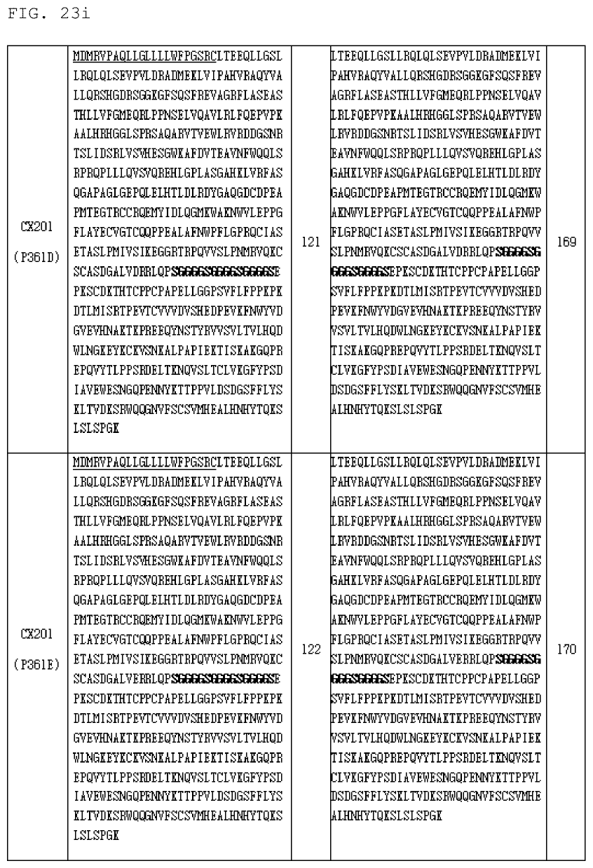

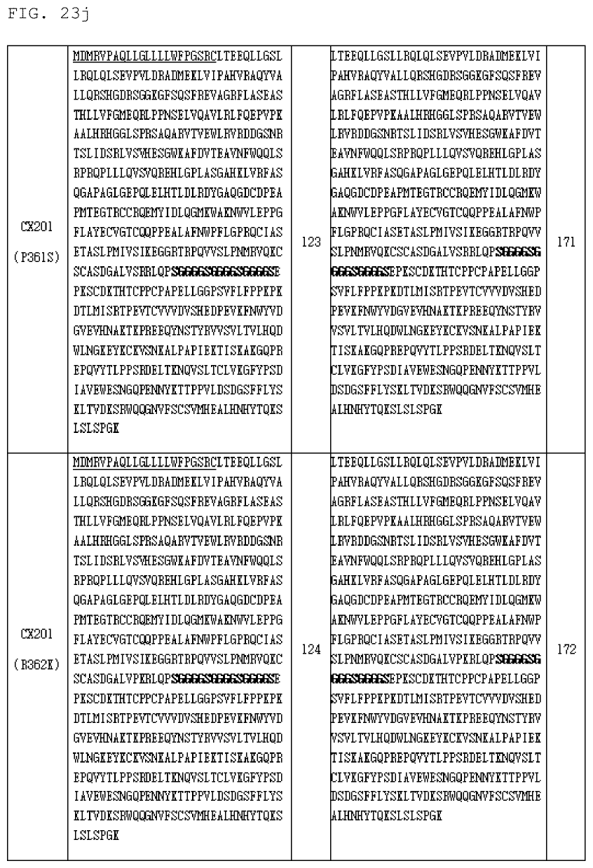

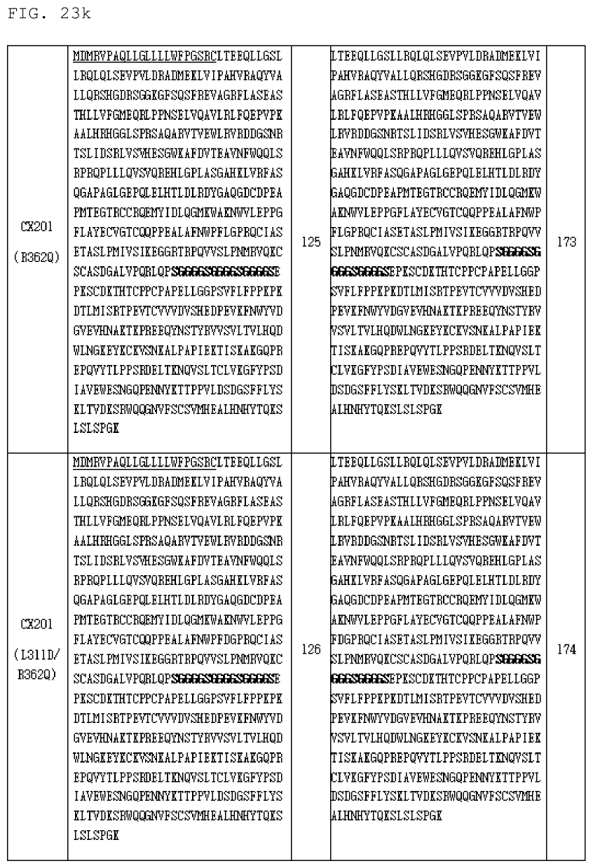

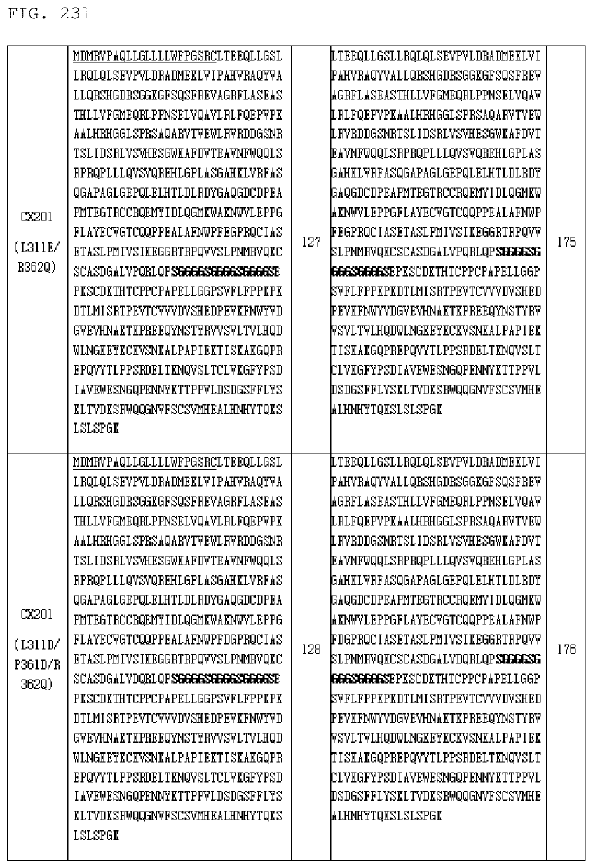

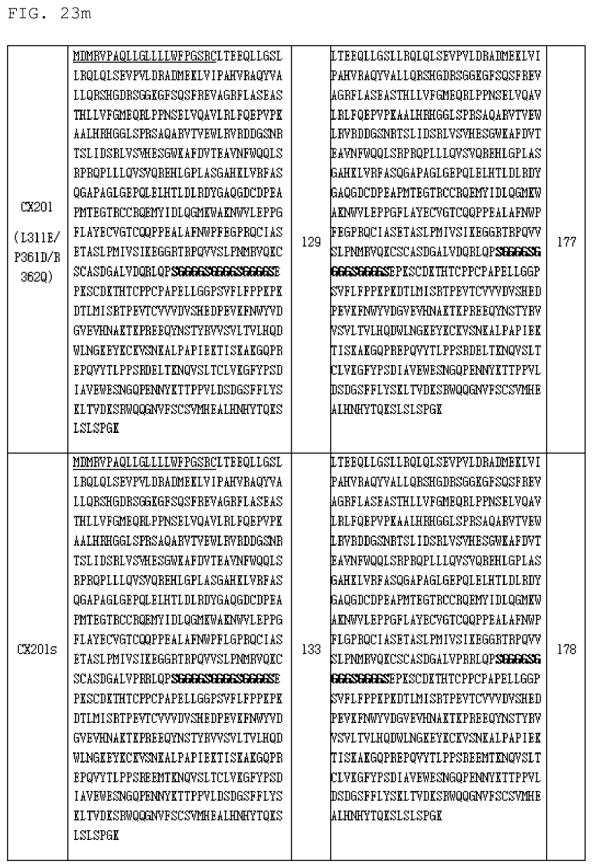

In the present invention, the amino acid sequences set forth in SEQ ID NOS: 134 to 159 refer to the amino acid sequences of L22 to K614 except for the signal sequence in the amino acid sequences set forth in SEQ ID NO: 86 to SEQ ID NO: 111, respectively. The amino acid sequences set forth in SEQ ID NOS: 160 to 178 refer to the amino acid sequences of L23 to K615 except for the signal sequence in the amino acid sequences set forth in SEQ ID NOS: 112 to 129 and SEQ ID NO: 133, respectively (FIGS. 22a to 22j and FIGS. 23a to 23m).

The fusion protein according to the present invention may further comprise a signal peptide at the N-terminus. The signal peptide capable of binding to the N-terminus of the fusion protein may be a signal sequence (M1 to A21) derived from the Lefty A protein, similar to that of the human Lefty A protein variant, but is not limited thereto. The signal peptide that is used in the present invention may be a signal sequence derived from a protein other than the Lefty A protein, for example, an antibody-derived sequence such as MDMRVPAQLLGLLLLWFPGSRC (UniProt: A0A0C4DH73; SEQ ID NO: 132), but is not limited thereto.

In still another aspect, the present invention is directed to a nucleic acid molecule encoding the fusion protein, an expression vector comprising the nucleic acid molecule, a recombinant cell into which the expression vector has been introduced, and a method of producing a fusion protein comprising a human Lefty A protein variant using the recombinant cell.

As used herein, the term “nucleic acid molecule” is meant to comprehensively include DNA (gDNA and cDNA) and RNA molecules, and nucleotide, which is the basic unit of a nucleic acid molecule, also includes sugar or base-modified analogues, as well as natural nucleotide (Scheit, Nucleotide Analogs, John Wiley, New York(1980); Uhlman and Peyman, Chemical Reviews. 1990; 90:543-84). The sequence of the nucleic acid molecule encoding the human Lefty A fusion protein variant of the present invention may be modified. The modification includes the addition, deletion, or non-conservative substitution or conservative substitution of nucleotides.

The term “vector” as used herein, includes a plasmid vector; a cosmid vector; a bacteriophage vector; and a viral vector, e.g., an adenovirus vector, retroviral vectors, and adeno-associated viral vectors as a mean for expressing a target gene in a host cell. Preferably, the vector may include a plasmid vector, but is not limited thereto.

The nucleic acid molecule encoding the fusion protein comprising the human Lefty A protein variant in the vector of the present invention may be operably linked to a promoter.

As used herein, the term “operably linked” refers to a functional linkage between a nucleic acid expression control sequence (e.g., an array of promoter, signal sequence, or transcription regulation factor binding site) and another nucleic acid sequence, and thus the control sequence controls the transcription and/or translation of the other nucleic acid sequence.

The recombinant vector system of the present invention may be constructed by various methods known in the pertinent art, and the detailed method thereof is disclosed in Sambrook et al., Molecular Cloning, A Laboratory Manual, Cold Spring Harbor Laboratory Press (2001), which is incorporated herein by reference.

The vector may be typically constructed as a vector for cloning or a vector for expression. The vector may be constructed as a vector that employs a prokaryotic cell or a eukaryotic cell as a host.

For example, when the vector of the present invention is an expression vector, and a prokaryotic cell is used as a host cell, a strong promoter capable of promoting transcription (such as tac promoter, lac promoter, lacUV5 promoter, 1pp promoter, pLλ promoter, pRλ promoter, racy promoter, amp promoter, recA promoter, SP6 promoter, trp promoter, T7 promoter, and the like), a ribosome-binding site for initiation of translation, and a transcription/translation termination sequence are generally included. As a host cell, when E. coli such as HB101, BL21, DH5α and the like is used, an operator and promoter for E. coli tryptophan biosynthesis (Yanofsky, C, J. Bacteriol., (1984) 158:1018-1024) and a phage λ left promoter (pLλ promoter, Herskowitz, I. and Hagen, D, Ann. Rev. Genet, (1980) 14:399-445) may be used as a regulatory sequence. When bacilli are used as host cells, the promoter for a toxin (protein) gene from Bacillus thuringiensis (Appl. Environ. Microbiol. (1998) 64:3932-3938; Mol. Gen. Genet. (1996) 250:734-741) or any promoters which can be expressed in bacilli may be used as a regulatory sequence.

Meanwhile, the expression vector of the present invention may be constructed by manipulating plasmids (e.g., pCL, pSC101, pGV1106, pACYC177, ColE1, pKT230, pME290, pBR322, pUC8/9, pUC6, pBD9, pHC79, pIJ61, pLAFR1, pHV14, pGEX series, pET series, pUC19, etc.), phages (e.g., λgt·4λB, λ-Charon, λΔz1, M13, etc.), or viruses (e.g., SV40, etc.), which are commonly used in the art. For example, the expression vector of the present invention may be constructed by manipulating a pCL expression vector, specifically a pCLS05 (Korean Patent Registration No. 10-1420274) expression vector, but is not limited thereto.

In addition, when the vector of the present invention is an expression vector, and an eukaryotic cell is used as a host cell, promoters derived from genomes of mammalian cells (e.g., a metallothionein promoter, a β-actin promoter, a human hemoglobin promoter and a human muscle creatinine promoter) or promoters derived from mammalian viruses (e.g., an adenovirus late promoter, a vaccinia virus 7.5K promoter, an SV40 promoter, a cytomegalovirus (CMV) promoter, a tk promoter of HSV, a promoter of mouse mammary tumor virus (MMTV), an LTR promoter of HIV, a promoter of moloney virus, a promoter of Epstein Barr Virus (EBV), and a promoter of Rous Sarcoma Virus (RSV) may be used. And the vector generally includes a polyadenylated sequence as a transcriptional termination sequence. Specifically, the recombinant vector of the present invention includes a CMV promoter.

The recombinant vector of the present invention may be fused with another sequence in order to facilitate purification of a recombinant protein expressed therefrom. The fused sequence includes, for example, glutathione S-transferase (Pharmacia, USA), maltose-binding protein (NEB, USA), FLAG (IBI, USA), 6×His (hexahistidine; Quiagen, USA), and the like. In addition, since Fc is fused to the protein expressed by the vector of the present invention, the expressed protein may be easily purified by a protein A column or the like without requiring an additional sequence for the purification.

Meanwhile, the recombinant vector of the present invention includes an antibiotic resistance gene commonly used in the art as a selective marker, and may include, for example, genes having resistance to ampicillin, gentamicin, carbenicillin, chloramphenicol, streptomycin, kanamycin, geneticin, neomycin, and tetracycline.

A recombinant cell, which is capable of stably and consecutively cloning and expressing the vector of the present invention, may be used as any host cells known in the art. The host cell includes the prokaryotic host cell, for example, such as a strain belonging to the genus Bacillus such as Escherichia coli, Bacillus subtilis, and Bacillus thuringiensis, Streptomyces, Pseudomonas (for example, Pseudomonas putida), Proteus mirabilis, and Staphylococcus (for example, Staphylococcus carnosus), but is not limited thereto.

Eukaryotic host cells which are suitable to be used with the vector include fugi such as Aspergillus sp. and yeast such as Pichia pastoris, Saccharomyces cerevisiae, Schizosaccharomyces and Neurospora crassa and other lower eukaryotic cells, and higher eukaryotic cells such as insect-derived cells, and cells derived from plants and mammals.

Specifically, the host cells may be monkey kidney cells (C0S7), NSO cells, SP2/0, Chinese hamster ovary (CHO) cells, W138, baby hamster kidney (BHK) cells, MDCK, myeloma cells, HuT 78 cells or HEK293 cells.

The use of a microorganism such as E. coli has higher productivity than that of animal cells or the like, but is not preferable for protein production due to problems such as disulfide bond formations or glycosylation. However, this microorganism may be used for production for the purpose of increasing the in vivo stability of a drug by pegylation or the like.

In the present invention, transfection or transformation into a host cell includes any method by which nucleic acids can be introduced into organisms, cells, tissues or organs, and, as known in the art, may be performed using a suitable standard technique selected according to the kind of host cell. These methods include, but are not limited to, electroporation, protoplast fusion, calcium phosphate (CaPO₄) precipitation, calcium chloride (CaCl₂) precipitation, agitation with silicon carbide fiber, and agrobacterium-, PEG-, dextran sulfate-, lipofectamine- and desiccation/inhibition-mediated transformation.

The present invention provides a method of producing a recombinant protein using the recombinant cell. Specifically, the method may be a method for producing a human Lefty A fusion protein variant, comprising the steps of: (a) culturing a recombinant cell transformed with the recombinant vector of the present invention; and (b) expressing the recombinant protein in the recombinant cell.

The culturing step in the production of the recombinant protein can be performed using a suitable medium and culture conditions known in the art. A person skilled in the art would be able to modify the culture conditions according to the particular strains selected without difficulty. These culutue methods are disclosed in various documents (e.g., James M. Lee, Biochemical Engineering, Prentice-Hall International Editions, 138-176). Methods for culturing cells may be divided into a suspension culture and an adherent culture based on the cell growth mode, and into a batch method, a fed-batch method and a continuous method according to the culture mode. The media employed for the culture should be selected to appropriately meet the conditions required by the particular strains employed.

In animal cell culture, the medium includes a carbon source, a nitrogen source, and a trace element component. Examples of the carbon source that can be used in the present invention include carbohydrates such as glucose, sucrose, lactose, fructose, maltose, starch and cellulose, fat such as soybean oil, sunflower seed oil, castor oil and coconut oil, fatty acid such as palmitic acid, stearic acid and linoleic acid, alcohol such as glycerol and ethanol, and organic acid such as acetic acid. These carbon sources may be used alone or in combination of two or more thereof.

The nitrogen source that can be used in the present invention includes, for example, an organic nitrogen source such as peptone, yeast extract, gravy, malt extract, corn steep liquor (CSL), and soybean meal powder, and an inorganic nitrogen source such as urea, ammonium sulfate, ammonium chloride, ammonium phosphate, ammonium carbonate and ammonium nitrate, and these nitrogen sources may be used alone or in combination of two or more thereof. The medium may include, as a phosphate source, potassium dihydrogen phosphate, dipotassium hydrogen phosphate, and sodium-containing salt corresponding thereof. In addition, as the phosphate source, metal salts such as magnesium sulfate or iron sulfate may be included in the medium. Besides, amino acids, vitamins and proper precursors may be included in the medium.

During cell culture, the pH of cell culture medium may be adjusted by adding compounds, such as ammonium hydroxide, potassium hydroxide, ammonia, phosphoric acid and sulfuric acid, to the culture medium in a suitable manner. In addition, during the culture process, an antifoaming agent such as fatty acid polyglycol ester may be used to inhibit bubble generation. Furthermore, oxygen or oxygen-containing gas (e.g., air) is injected into the culture medium to keep the culture aerobic. The temperature of the culture is usually 20° C. to 45° C., preferably 25° C. to 40° C.

The recombinant protein obtained by culturing a transformed recombinant cell may be used without purification, or may be used after purifying the same to high purity by various conventional methods, for example, dialysis, salt precipitation and chromatography. Among these methods, the chromatography-based method is most frequently used, and the type and sequence of chromatography may be selected from among ion-exchange chromatography, size-exclusion chromatography, affinity chromatography and the like, depending on the characteristics of the recombinant protein, the culture method, etc.

In yet another aspect, the present invention is directed to a composition for preventing and/or treating neuromuscular disease, which comprises either the human Lefty A protein variant or the fusion protein.

In a further aspect, the present invention is directed to a method for preventing and/or treating neuromuscular disease, which comprises administering either the human Lefty A protein variant or the fusion protein comprising the human Lefty A protein variant to a subject.

In a still further aspect, the present invention is directed to a use of either the human Lefty A protein variant or the fusion protein comprising the human Lefty A protein variant, for the prevention and/or treatment of neuromuscular disease.

In a yet further aspect, the present invention is directed to a use of either the human Lefty A protein variant or the fusion protein comprising the human Lefty A protein variant, for the manufacture of a medicament for treating neuromuscular disease.

As used herein, the term “preventing/prevention” means any action that inhibits or delays progress of diseases such as neuromuscular disease and the like by administration of the composition according to the present invention, and “treating/treatment” means suppression of development, alleviation, or elimination of diseases such as neuromuscular disease and the like.

In one example of the present invention, it was confirmed that the fusion protein comprising the human Lefty A protein variant according to the present invention could bind to human Nodal and inhibit Nodal-induced signaling, thus improving peripheral neuropathy-associated parameters. In addition, in one example of the present invention, it was confirmed that the human Lefty A fusion protein variant dose-dependently can inhibit myostatin signaling. Furthermore, it was found that the human Lefty A fusion protein variant can block p38 signaling, a negative regulator of myelination, and thus may be used as an agent for treating peripheral neuropathy, especially neuropathy caused by demyelination.

Therefore, in the present invention, the neuromuscular disease may be a disease occurring in peripheral nerves or muscles, preferably a disease related to Nodal and/or myostatin signaling, but is not limited thereto. The Nodal and/or myostatin signaling-related disease may be myopathy, peripheral neuropathy, or rigid spine syndrome.

The myopathy may be selected from the group consisting of sarcopenia, muscular dystrophy, myasthenia gravis, amyotrophic lateral sclerosis (or Lou Gehrig's disease), primary lateral sclerosis, progressive muscular atrophy, Kennedy's disease (or spinobulbar muscular atrophy), spinal muscular atrophy and distal myopathy.

The peripheral neuropathy may be selected from the group consisting of Charcot-Marie-Tooth disease, chronic inflammatory demyelinating polyneuropathy, carpal tunnel syndrome, diabetic peripheral neuropathy and Guillain-Barre syndrome.

The composition may be in the form of a pharmaceutical composition, a quasi-drug composition or a health functional food composition.

The composition for preventing or treating disease of the present invention may further comprise a pharmaceutically acceptable carrier.

As used herein, the term “pharmaceutically acceptable carrier” refers to a carrier or diluent that does not impair the biological activity and characteristics of an administered compound without irritating an organism. As a pharmaceutically acceptable carrier in a composition that is formulated as a liquid solution, a sterile and biocompatible carrier is used. The pharmaceutically acceptable carrier may be physiological saline, sterile water, buffered saline, albumin injection solution, dextrose solution, maltodextrin solution, glycerol, or a mixture of two or more thereof. In addition, the composition of the present invention may, if necessary, comprise other conventional additives, including antioxidants, buffers, and bacteriostatic agents. Further, the composition of the present invention may be formulated as injectable forms such as aqueous solutions, suspensions or emulsions with the aid of diluents, dispersants, surfactants, binders and lubricants. In addition, the composition according to the present invention may be formulated in the form of pills, capsules, granules, or tablets.

The pharmaceutical composition according to the present invention may be formulated in an oral or parenteral dosage form. The pharmaceutical composition according to the present invention is formulated using diluents or excipients, such as fillers, extenders, binders, wetting agents, disintegrants or surfactants, which are commonly used. Solid formulations for oral administration include tablets, pills, powders, granules, capsules, etc. Such solid formulations are prepared by mixing one or more compounds with at least one excipient, such as starch, calcium carbonate, sucrose, lactose, gelatin, etc. In addition to simple expedients, lubricants such as magnesium stearate, talc, etc., may also be added. Liquid formulations for oral administration, such as suspensions, internal solutions, emulsions, syrups, etc., may include simple diluents, e.g., water and liquid paraffin, as well as various excipients, e.g., wetting agents, sweeteners, aromatics, preservatives, etc. Formulations for parenteral administration include sterilized aqueous solutions, non-aqueous solvents, suspensions, emulsions, lyophilized agents, and suppositories. Non-aqueous solvents and suspensions may be prepared using propylene glycol, polyethylene glycol, vegetable oils such as olive oil, or injectable esters such as ethyloleate. As a base for suppositories, Witepsol, Macrogol, Tween 61, cacao fat, laurin fat, glycerogelatin, etc. may be used.

The pharmaceutically acceptable carrier and formulations are disclosed in detail in Remington's Pharmaceutical Sciences (19th ed., 1995).

The pharmaceutical composition of the present invention may be administered orally or parenterally. The parenteral administration is carried out by intravenous injection, subcutaneous injection, intramuscular injection, intraperitoneal injection, endothelial administration, topical administration, intranasal administration, intrapulmonary administration, rectal administration, and the like. For the oral administration, the active ingredient in the composition needs to be formulated into a coated dosage form or into a dosage form which can be protected the active ingredient from being disintegrated in stomach considering that peptides and proteins are digested in stomach. Alternatively, the composition of the present invention may be administered via any device by which the active ingredient can move to the target cell of interest.

The appropriate dosage of the composition for preventing or treating disease according to the present invention may vary depending on factors such as the formulation method, the administration method, patient's age, body weight, sex, pathological condition, food, administration time, route of administration, excretion rate and reaction sensitivity. Thus, a commonly skilled physician can easily determine and prescribe a dosage that is effective for the desired treatment or prevention of disease of interest.

According to one embodiment of the present invention, the daily dosage of the pharmaceutical composition of the present invention may be 0.001 mg/kg to 100 mg/kg. The term “pharmaceutically effective amount” as used herein refers to an amount sufficient to prevent, treat and diagnose diseases such as neuromuscular disease and the like.

The composition for preventing or treating disease of the present invention may be formulated using a pharmaceutically acceptable carrier and/or an excipient according to a method which can be easily carried out by those having ordinary skill in the art to which the present invention pertains so as to be provided in a unit dosage form or enclosed into multi-dose vials. Here, the formulations may be in the form of solutions, suspensions or emulsions in oils or aqueous media, or in the form of extracts, grains, suppositories, powders, granules, tablets or capsules, and may additionally include dispersing or stabilizing agents.

Preferably, the composition of the present invention can be formulated in an injectable form, in which case, the injectable formulation may be either a reconstituted lyophilized formulation or a liquid formulation provided in a ready-to-inject (RTI) form, but is not limited thereto.

The composition of the present invention may be administered as an individual therapeutic agent or in combination with another therapeutic agent, and may be administered sequentially or simultaneously with a conventional therapeutic agent.

### EXAMPLES

Hereinafter, the present invention will be described in further detail with reference to examples. It will be obvious to a person having ordinary skill in the art that these examples are for illustrative purposes only and are not to be construed to limit the scope of the present invention.

### Example 1: Construction of Human Lefty A Fusion Proteins

### Example 1-1: Construction of Human Lefty A Protein Variants

The amino acid sequence of wild-type human Lefty A protein is as follows:

(SEQ ID NO: 131)
MWPLWLCWAL WVLPLAGPGA ALTEEQLLGS LLRQLQLSEV
PVLDRADMEK LVIPAHVRAQ YVVLLRRSHG DRSRGKRFSQ
SFREVAGRFL ASEASTHLLV FGMEQRLPPN SELVQAVLRL
FQEPVPKAAL HRHGRLSPRS AQARVTVEWL RVRDDGSNRT
SLIDSRLVSV HESGWKAFDV TEAVNFWQQL SRPRQPLLLQ
VSVQREHLGP LASGAHKLVR FASQGAPAGL GEPQLELHTL
DLRDYGAQGD CDPEAPMTEG TRCCRQEMYI DLQGMKWAKN
WVLEPPGFLA YECVGTCQQP PEALAFNWPF LGPRQCIASE
TASLPMIVSI KEGGRTRPQV VSLPNMRVQK CSCASDGALV
PRRLQP

In the amino acid sequence, M1 to A21 correspond to a signal peptide sequence, L22 to S73 correspond to a propeptide domain, and R74 to R77 and R132 to R135 correspond to processing sites.

The wild-type human Lefty A protein is a 42 kDa form before processing, but is processed into a 34 kDa or 28 kDa form while a region comprising the propeptide domain is removed by proprotein convertases. In order to elucidate the function of these three forms of protein fragment, 42, 34 and 28 kDa protein expression vectors were constructed.

For the 42 kDa and 34 kDa form proteins, processing site mutations (42: R74G/R77G/R132G, 34: R132G) were introduced during construction in order to prevent additional cleavage (THE JOURNAL OF BIOLOGICAL CHEMISTRY, Vol. 276, No. 24, Issue of June 15, pp. 21387-21396, 2001). Human Lefty A 42 fragment (L22 to P366, R74G/R77G/R132G), 34 fragment (F78 to P366, R132G) and 28 fragment (L136 to P366) genes were synthesized by Bioneer Co., Ltd. (Korea) and used as PCR templates. Using a primer pair of L1_F (SEQ ID NO: 13) and L2R (SEQ ID NO: 14) and a ProFlex system (Applied Biosystems, USA), an amplification process was performed with Ex taq (Takara, Japan) according to the manufacturer's instruction under the following conditions, thereby obtaining human Lefty A 42, 34 and 28 fragments: 22 cycles, each consisting of denaturation at 95° C. for 60 sec, primer annealing at 58° C. for 60 sec, and extension at 72° C. for 60 sec.

### Example 1-2: Construction of C-Terminal Fc Fusion Proteins

Three expression vectors of C-terminal Fc fusion proteins (42 Fc (SEQ ID NO: 1), 34 Fc (SEQ ID NO: 2) and 28 Fc (SEQ ID NO: 3)) were constructed by linking human IgG1 Fc to the human Lefty A protein variants of Example 1-1. The 28 Fc, 34 Fc and 42 Fc mean that Fc is fused at the C-terminus of each of 28, 34 and 42 kDa Lefty A proteins.

Using DNA encoding the human IgG1 (Uniprot: P01857) sequence as a template and a primer pair of L3_F (SEQ ID NO: 15) and L4R (SEQ ID NO: 16), amplification was performed under the same conditions as those used for the PCR amplification of the human Lefty A fragments, thereby obtaining human IgG1 fragments. The obtained PCR products were separated and purified by 1.5% agarose gel electrophoresis, and then subjected to an assembly PCR reaction under the same conditions. The obtained reaction products were separated and purified by 1.5% agarose gel electrophoresis, and then cleaved using the restriction enzymes Hind III and Xho I (NEB, USA). Each of the cleavage products was ligated with a pCLS05 vector (Korean Patent Application No. 2011-0056685), cleaved with the restriction enzymes Hind III and Xho I, at 25° C. for 60 minutes, and then the ligation products were transformed into E. coli DH5α. The transformed cells were cultured overnight in LB medium containing 100 μg/ml of ampicillin, and plasmids were extracted from the produced colonies, and then sequenced by the service of Cosmo Genetech Co., Ltd. (Korea), thereby confirming that three C-terminal Fc fusion protein expression vectors were constructed.

### Example 1-3: Construction of C-Terminal Linker Fc Fusion Proteins

Three expression vectors of C-terminal linker Fc fusion proteins (42 LFc (SEQ ID NO: 4), 34 LFc (SEQ ID NO: 5) and 28 LFc (SEQ ID NO: 6)) were constructed by linking human IgG1 Fc to human Lefty A via a SGGGGSGGGGSGGGGS linker (SEQ ID NO: 130). The 28 LFc, 34 LFc and 42 LFc mean that Fc is fused at the C-terminus of each of 28, 34 and 42 kDa Lefty A proteins via a linker.

Human Lefty A 42, 34 and 28 fragments were obtained in the same manner as described for the construction of the C-terminal Fc fusion protein expression vectors. For linker Fc fragments, using DNA encoding the human IgG1 (Uniprot: P01857) sequence as a template and a primer pair of L5_F (SEQ ID NO: 17) and L4_R (SEQ ID NO: 16), PCR amplification was performed under the same conditions, thereby obtaining human linker IgG1 fragments. The subsequent procedure was the same as described above with respect to the construction of the C-terminal Fc fusion proteins.

### Example 1-4: Construction of N-Terminal Fc Fusion Proteins

Three expression vectors of N-terminal Fc fusion proteins (Fc 42 (SEQ ID NO: 7), Fc 34 (SEQ ID NO: 8) and Fc 28 (SEQ ID NO: 9)) were constructed. The Fc 28, Fc 34 and Fc 42 mean that Fc is fused at the N-terminus of each of 28, 34 and 42 kDa Lefty A proteins.

Using primer pairs of L6_F (SEQ ID NO: 18), L7_F (SEQ ID NO: 19), L8_F (SEQ ID NO: 20) and L9_R (SEQ ID NO: 21), human Lefty A 42, 34 and 28 fragments were obtained. Each of the obtained reaction products was separated and purified by 1.5% agarose gel electrophoresis, and then ligated with a human IgG1 Fc-encoding pCLS05 vector DNA, digested with the restriction enzyme Xho I, using an In-Fusion® HD Cloning Kit (Clontech, 639650) at 50° C. for 15 minutes. The subsequent procedure was the same as described above with respect to the construction of the C-terminal Fc fusion proteins.

### Example 1-5: Construction of C-Terminal HSA Fusion Proteins

A human IgG1 Fc fusion protein forms a homodimer when expressed in animal cells. For this reason, in order to construct monomeric fusion proteins, human serum albumin (HSA)-fused proteins were constructed. Three expression vectors of C-terminal HSA fusion proteins (42 HSA (SEQ ID NO: 10), 34 HSA (SEQ ID NO: 11) and 28 HSA (SEQ ID NO: 12)) were constructed. The 28 HSA, 34 HSA and 42 HSA mean that HSA is fused at the C-terminus of each of 28, 34 and 42 kDa Lefty A proteins.

Using a primer pair of L1_F (SEQ ID NO: 13) and L9_R (SEQ ID NO: 21), Lefty A 42, 34 and 28 fragments were obtained in the same manner as Example 1. Each of the fragments were cleaved with the restriction enzymes Hind III and Xho I and ligated with a pCLS05 vector, ligated with Hind III and Xho I, at 25° C. for 60 minutes. The ligation products were transformed into E. coli DH5α. The transformed cells were cultured overnight in LB medium containing 100 μg/ml of ampicillin, and plasmids were extracted from the produced colonies, and then sequenced, thereby confirming the first-step cloning.

In second-step cloning, using the human serum albumin gene synthesized by Bioneer as a template and a primer pair of L10_F (SEQ ID NO: 22) and L11_R (SEQ ID NO: 23), human serum albumin-containing fragments were obtained under the same PCR conditions as used in Example 1. The obtained PCR products were separated and purified by 1.5% agarose gel electrophoresis, and then ligated with DNAs (encoding the human Lefty A 42, 34 and 28 fragments obtained in the first-step cloning), cleaved with the restriction enzyme Xho I, using the In-Fusion® HD Cloning Kit (Clontech, 639650) at 50° C. for 15 minutes. The subsequent procedure was the same as described above with respect to the construction of the C-terminal Fc fusion proteins.

Schematic diagram of the expression vectors constructed in Example 1-1 to Example 1-5 is shown in FIG. 1, the amino acid sequences thereof are shown in Table 1 below, and the primers used in the construction of the fusion proteins are shown in Table 2 below.

TABLE 1
Amino acid sequences of human Lefty A
fusion proteins

| | | SEQ |
|---|---|---|
| | | ID |
| | Amino acid sequences | NOS: |
| 42 Fc | MWPLWLCWALWVLPLAGPGAALTEEQLLGSLLRQLQLSEVPVLDR | 1 |
| | ADMEKLVIPAHVRAQYVVLLRRSHGDRSGGKGFSQSFREVAGRFL | |
| | ASEASTHLLVFGMEQRLPPNSELVQAVLRLFQEPVPKAALHGHGR | |
| | LSPRSAQARVTVEWLRVRDDGSNRTSLIDSRLVSVHESGWKAFDV | |
| | TEAVNFWQQLSRPRQPLLLQVSVQREHLGPLASGAHKLVRFASQG | |
| | APAGLGEPQLELHTLDLRDYGAQGDCDPEAPMTEGTRCCRQEMYI | |
| | DLQGMKWAKNWVLEPPGFLAYECVGTCQQPPEALAFNWPFLGPRQ | |
| | CIASETASLPMIVSIKEGGRTRPQVVSLPNMRVQKCSCASDGALV | |
| | PRRLQPEPKSCDKTHTCPPCPAPELLGGPSVFLFPPKPKDTLMIS | |
| | RTPEVTCVVVDVSHEDPEVKFNWYVDGVEVHNAKTKPREEQYNST | |
| | YRVVSVLTVLHQDWLNGKEYKCKVSNKALPAPIEKTISKAKGQPR | |
| | EPQVYTLPPSRDELTKNQVSLTCLVKGFYPSDIAVEWESNGQPEN | |
| | NYKTTPPVLDSDGSFFLYSKLTVDKSRWQQGNVFSCSVMHEALHN | |
| | HYTQKSLSLSPGK | |

| | | |
|---|---|---|
| 34 Fc | MWPLWLCWALWVLPLAGPGAAFSQSFREVAGRFLASEASTHLLVF | 2 |
| | GMEQRLPPNSELVQAVLRLFQEPVPKAALHGHGRLSPRSAQARVT | |
| | VEWLRVRDDGSNRTSLIDSRLVSVHESGWKAFDVTEAVNFWQQLS | |
| | RPRQPLLLQVSVQREHLGPLASGAHKLVRFASQGAPAGLGEPQLE | |
| | LHTLDLRDYGAQGDCDPEAPMTEGTRCCRQEMYIDLQGMKWAKNW | |
| | VLEPPGFLAYECVGTCQQPPEALAFNWPFLGPRQCIASETASLPM | |
| | IVSIKEGGRTRPQVVSLPNMRVQKCSCASDGALVPRRLQPEPKSC | |
| | DKTHTCPPCPAPELLGGPSVFLFPPKPKDTLMISRTPEVTCVVVD | |
| | VSHEDPEVKFNWYVDGVEVHNAKTKPREEQYNSTYRVVSVLTVLH | |
| | QDWLNGKEYKCKVSNKALPAPIEKTISKAKGQPREPQVYTLPPSR | |
| | DELTKNQVSLTCLVKGFYPSDIAVEWESNGQPENNYKTTPPVLDS | |
| | DGSFFLYSKLTVDKSRWQQGNVFSCSVMHEALHNHYTQKSLSLSP | |
| | GK | |
| 28 Fc | MWPLWLCWALWVLPLAGPGAALSPRSAQARVTVEWLRVRDDGSNR | 3 |
| | TSLIDSRLVSVHESGWKAFDVTEAVNFWQQLSRPRQPLLLQVSVQ | |
| | REHLGPLASGAHKLVRFASQGAPAGLGEPQLELHTLDLRDYGAQG | |
| | DCDPEAPMTEGTRCCRQEMYIDLQGMKWAKNWVLEPPGFLAYECV | |
| | GTCQQPPEALAFNWPFLGPRQCIASETASLPMIVSIKEGGRTRPQ | |
| | VVSLPNMRVQKCSCASDGALVPRRLQPEPKSCDKTHTCPPCPAPE | |
| | LLGGPSVFLFPPKPKDTLMISRTPEVTCVVVDVSHEDPEVKFNWY | |
| | VDGVEVHNAKTKPREEQYNSTYRVVSVLTVLHQDWLNGKEYKCKV | |
| | SNKALPAPIEKTISKAKGQPREPQVYTLPPSRDELTKNQVSLTCL | |
| | VKGFYPSDIAVEWESNGQPENNYKTTPPVLDSDGSFFLYSKLTVD | |
| | KSRWQQGNVFSCSVMHEALHNHYTQKSLSLSPGK | |
| 42 LFc | MWPLWLCWALWVLPLAGPGAALTEEQLLGSLLRQLQLSEVPVLDR | 4 |
| | ADMEKLVIPAHVRAQYVVLLRRSHGDRSGGKGFSQSFREVAGRFL | |
| | ASEASTHLLVFGMEQRLPPNSELVQAVLRLFQEPVPKAALHGHGR | |
| | LSPRSAQARVTVEWLRVRDDGSNRTSLIDSRLVSVHESGWKAFDV | |
| | TEAVNFWQQLSRPRQPLLLQVSVQREHLGPLASGAHKLVRFASQG | |
| | APAGLGEPQLELHTLDLRDYGAQGDCDPEAPMTEGTRCCRQEMYI | |
| | DLQGMKWAKNWVLEPPGFLAYECVGTCQQPPEALAFNWPFLGPRQ | |
| | CIASETASLPMIVSIKEGGRTRPQVVSLPNMRVQKCSCASDGALV | |
| | PRRLQPSGGGGSGGGGSGGGGSEPKSCDKTHTCPPCPAPELLGGP | |
| | SVFLFPPKPKDTLMISRTPEVTCVVVDVSHEDPEVKFNWYVDGVE | |
| | VHNAKTKPREEQYNSTYRVVSVLTVLHQDWLNGKEYKCKVSNKAL | |
| | PAPIEKTISKAKGQPREPQVYTLPPSRDELTKNQVSLTCLVKGFY | |
| | PSDIAVEWESNGQPENNYKTTPPVLDSDGSFFLYSKLTVDKSRWQ | |
| | QGNVFSCSVMHEALHNHYTQKSLSLSPGK | |
| 34 LFc | MWPLWLCWALWVLPLAGPGAAFSQSFREVAGRFLASEASTHLLVF | 5 |
| | GMEQRLPPNSELVQAVLRLFQEPVPKAALHGHGRLSPRSAQARVT | |
| | VEWLRVRDDGSNRTSLIDSRLVSVHESGWKAFDVTEAVNFWQQLS | |
| | RPRQPLLLQVSVQREHLGPLASGAHKLVRFASQGAPAGLGEPQLE | |
| | LHTLDLRDYGAQGDCDPEAPMTEGTRCCRQEMYIDLQGMKWAKNW | |
| | VLEPPGFLAYECVGTCQQPPEALAFNWPFLGPRQCIASETASLPM | |
| | IVSIKEGGRTRPQVVSLPNMRVQKCSCASDGALVPRRLQPSGGGG | |
| | SGGGGSGGGGSEPKSCDKTHTCPPCPAPELLGGPSVFLFPPKPKD | |
| | TLMISRTPEVTCVVVDVSHEDPEVKFNWYVDGVEVHNAKTKPREE | |
| | QYNSTYRVVSVLTVLHQDWLNGKEYKCKVSNKALPAPIEKTISKA | |
| | KGQPREPQVYTLPPSRDELTKNQVSLTCLVKGFYPSDIAVEWESN | |
| | GQPENNYKTTPPVLDSDGSFFLYSKLTVDKSRWQQGNVFSCSVMH | |
| | EALHNHYTQKSLSLSPGK | |
| 28 LFc | MWPLWLCWALWVLPLAGPGAALSPRSAQARVTVEWLRVRDDGSNR | 6 |
| | TSLIDSRLVSVHESGWKAFDVTEAVNFWQQLSRPRQPLLLQVSVQ | |
| | REHLGPLASGAHKLVRFASQGAPAGLGEPQLELHTLDLRDYGAQG | |
| | DCDPEAPMTEGTRCCRQEMYIDLQGMKWAKNWVLEPPGFLAYECV | |
| | GTCQQPPEALAFNWPFLGPRQCIASETASLPMIVSIKEGGRTRPQ | |
| | VVSLPNMRVQKCSCASDGALVPRRLQPSGGGGSGGGGSGGGGSEP | |
| | KSCDKTHTCPPCPAPELLGGPSVFLFPPKPKDTLMISRTPEVTCV | |
| | VVDVSHEDPEVKFNWYVDGVEVHNAKTKPREEQYNSTYRVVSVLT | |
| | VLHQDWLNGKEYKCKVSNKALPAPIEKTISKAKGQPREPQVYTLP | |
| | PSRDELTKNQVSLTCLVKGFYPSDIAVEWESNGQPENNYKTTPPV | |
| | LDSDGSFFLYSKLTVDKSRWQQGNVFSCSVMHEALHNHYTQKSLS | |
| | LSPGK | |
| Fc 42 | MGWSCIILFLVATATGVHSEPKSCDKTHTCPPCPAPELLGGPSVF | 7 |
| | LFPPKPKDTLMISRTPEVTCVVVDVSHEDPEVKFNWYVDGVEVHN | |
| | AKTKPREEQYNSTYRVVSVLTVLHQDWLNGKEYKCKVSNKALPAP | |
| | IEKTISKAKGQPREPQVYTLPPSRDELTKNQVSLTCLVKGFYPSD | |
| | IAVEWESNGQPENNYKTTPPVLDSDGSFFLYSKLTVDKSRWQQGN | |
| | VFSCSVMHEALHNHYTQKSLSLSPGKLTEEQLLGSLLRQLQLSEV | |
| | PVLDRADMEKLVIPAHVRAQYVVLLRRSHGDRSRGKRFSQSFREV | |
| | AGRFLASEASTHLLVFGMEQRLPPNSELVQAVLRLFQEPVPKAAL | |
| | HRHGRLSPRSAQARVTVEWLRVRDDGSNRTSLIDSRLVSVHESGW | |
| | KAFDVTEAVNFWQQLSRPRQPLLLQVSVQREHLGPLASGAHKLVR | |
| | FASQGAPAGLGEPQLELHTLDLRDYGAQGDCDPEAPMTEGTRCCR | |
| | QEMYIDLQGMKWAKNWVLEPPGFLAYECVGTCQQPPEALAFNWPF | |
| | LGPRQCIASETASLPMIVSIKEGGRTRPQVVSLPNMRVQKCSCAS | |
| | DGALVPRRLQP | |
| Fc 34 | MGWSCIILFLVATATGVHSEPKSCDKTHTCPPCPAPELLGGPSVF | 8 |
| | LFPPKPKDTLMISRTPEVTCVVVDVSHEDPEVKFNWYVDGVEVHN | |
| | AKTKPREEQYNSTYRVVSVLTVLHQDWLNGKEYKCKVSNKALPAP | |
| | IEKTISKAKGQPREPQVYTLPPSRDELTKNQVSLTCLVKGFYPSD | |
| | IAVEWESNGQPENNYKTTPPVLDSDGSFFLYSKLTVDKSRWQQGN | |
| | VFSCSVMHEALHNHYTQKSLSLSPGKFSQSFREVAGRFLASEAST | |
| | HLLVFGMEQRLPPNSELVQAVLRLFQEPVPKAALHRHGRLSPRSA | |
| | QARVTVEWLRVRDDGSNRTSLIDSRLVSVHESGWKAFDVTEAVNF | |
| | WQQLSRPRQPLLLQVSVQREHLGPLASGAHKLVRFASQGAPAGLG | |
| | EPQLELHTLDLRDYGAQGDCDPEAPMTEGTRCCRQEMYIDLQGMK | |
| | WAKNWVLEPPGFLAYECVGTCQQPPEALAFNWPFLGPRQCIASET | |
| | ASLPMIVSIKEGGRTRPQVVSLPNMRVQKCSCASDGALVPRRLQP | |
| Fc 28 | MGWSCIILFLVATATGVHSEPKSCDKTHTCPPCPAPELLGGPSVF | 9 |
| | LFPPKPKDTLMISRTPEVTCVVVDVSHEDPEVKFNWYVDGVEVHN | |
| | AKTKPREEQYNSTYRVVSVLTVLHQDWLNGKEYKCKVSNKALPAP | |
| | IEKTISKAKGQPREPQVYTLPPSRDELTKNQVSLTCLVKGFYPSD | |
| | IAVEWESNGQPENNYKTTPPVLDSDGSFFLYSKLTVDKSRWQQGN | |
| | VFSCSVMHEALHNHYTQKSLSLSPGKLSPRSAQARVTVEWLRVRD | |
| | DGSNRTSLIDSRLVSVHESGWKAFDVTEAVNFWQQLSRPRQPLLL | |
| | QVSVQREHLGPLASGAHKLVRFASQGAPAGLGEPQLELHTLDLRD | |
| | YGAQGDCDPEAPMTEGTRCCRQEMYIDLQGMKWAKNWVLEPPGFL | |
| | AYECVGTCQQPPEALAFNWPFLGPRQCIASETASLPMIVSIKEGG | |
| | RTRPQVVSLPNMRVQKCSCASDGALVPRRLQP | |
| 42 HSA | MWPLWLCWALWVLPLAGPGAALTEEQLLGSLLRQLQLSEVPVLDR | 10 |
| | ADMEKLVIPAHVRAQYVVLLRRSHGDRSGGKGFSQSFREVAGRFL | |
| | ASEASTHLLVFGMEQRLPPNSELVQAVLRLFQEPVPKAALHGHGR | |
| | LSPRSAQARVTVEWLRVRDDGSNRTSLIDSRLVSVHESGWKAFDV | |
| | TEAVNFWQQLSRPRQPLLLQVSVQREHLGPLASGAHKLVRFASQG | |
| | APAGLGEPQLELHTLDLRDYGAQGDCDPEAPMTEGTRCCRQEMYI | |
| | DLQGMKWAKNWVLEPPGFLAYECVGTCQQPPEALAFNWPFLGPRQ | |
| | CIASETASLPMIVSIKEGGRTRPQVVSLPNMRVQKCSCASDGALV | |
| | PRRLQPDAHKSEVAHRFKDLGEENFKALVLIAFAQYLQQCPFEDH | |
| | VKLVNEVTEFAKTCVADESAENCDKSLHTLFGDKLCTVATLRETY | |
| | GEMADCCAKQEPERNECFLQHKDDNPNLPRLVRPEVDVMCTAFHD | |
| | NEETFLKKYLYEIARRHPYFYAPELLFFAKRYKAAFTECCQAADK | |
| | AACLLPKLDELRDEGKASSAKQRLKCASLQKFGERAFKAWAVARL | |
| | SQRFPKAEFAEVSKLVTDLTKVHTECCHGDLLECADDRADLAKYI | |
| | CENQDSISSKLKECCEKPLLEKSHCIAEVENDEMPADLPSLAADF | |
| | VESKDVCKNYAEAKDVFLGMFLYEYARRHPDYSVVLLLRLAKTYE | |
| | TTLEKCCAAADPHECYAKVFDEFKPLVEEPQNLIKQNCELFEQLG | |
| | EYKFQNALLVRYTKKVPQVSTPTLVEVSRNLGKVGSKCCKHPEAK | |
| | RMPCAEDYLSVVLNQLCVLHEKTPVSDRVTKCCTESLVNRRPCFS | |
| | ALEVDETYVPKEFNAETFTFHADICTLSEKERQIKKQTALVELVK | |
| | HKPKATKEQLKAVMDDFAAFVEKCCKADDKETCFAEEGKKLVAAS | |
| | QAALGL | |
| 34 HSA | MWPLWLCWALWVLPLAGPGAAFSQSFREVAGRFLASEASTHLLVF | 11 |
| | GMEQRLPPNSELVQAVLRLFQEPVPKAALHGHGRLSPRSAQARVT | |
| | VEWLRVRDDGSNRTSLIDSRLVSVHESGWKAFDVTEAVNFWQQLS | |
| | RPRQPLLLQVSVQREHLGPLASGAHKLVRFASQGAPAGLGEPQLE | |
| | LHTLDLRDYGAQGDCDPEAPMTEGTRCCRQEMYIDLQGMKWAKNW | |
| | VLEPPGFLAYECVGTCQQPPEALAFNWPFLGPRQCIASETASLPM | |
| | IVSIKEGGRTRPQVVSLPNMRVQKCSCASDGALVPRRLQPDAHKS | |
| | EVAHRFKDLGEENFKALVLIAFAQYLQQCPFEDHVKLVNEVTEFA | |
| | KTCVADESAENCDKSLHTLFGDKLCTVATLRETYGEMADCCAKQE | |
| | PERNECFLQHKDDNPNLPRLVRPEVDVMCTAFHDNEETFLKKYLY | |
| | EIARRHPYFYAPELLFFAKRYKAAFTECCQAADKAACLLPKLDEL | |
| | RDEGKASSAKQRLKCASLQKFGERAFKAWAVARLSQRFPKAEFAE | |
| | VSKLVTDLTKVHTECCHGDLLECADDRADLAKYICENQDSISSKL | |
| | KECCEKPLLEKSHCIAEVENDEMPADLPSLAADFVESKDVCKNYA | |
| | EAKDVFLGMFLYEYARRHPDYSVVLLLRLAKTYETTLEKCCAAAD | |
| | PHECYAKVFDEFKPLVEEPQNLIKQNCELFEQLGEYKFQNALLVR | |
| | YTKKVPQVSTPTLVEVSRNLGKVGSKCCKHPEAKRMPCAEDYLSV | |
| | VLNQLCVLHEKTPVSDRVTKCCTESLVNRRPCFSALEVDETYVPK | |
| | EFNAETFTFHADICTLSEKERQIKKQTALVELVKHKPKATKEQLK | |
| | AVMDDFAAFVEKCCKADDKETCFAEEGKKLVAASQAALGL | |
| 28 HSA | MWPLWLCWALWVLPLAGPGAALSPRSAQARVTVEWLRVRDDGSNR | 12 |
| | TSLIDSRLVSVHESGWKAFDVTEAVNFWQQLSRPRQPLLLQVSVQ | |
| | REHLGPLASGAHKLVRFASQGAPAGLGEPQLELHTLDLRDYGAQG | |
| | DCDPEAPMTEGTRCCRQEMYIDLQGMKWAKNWVLEPPGFLAYECV | |
| | GTCQQPPEALAFNWPFLGPRQCIASETASLPMIVSIKEGGRTRPQ | |
| | VVSLPNMRVQKCSCASDGALVPRRLQPDAHKSEVAHRFKDLGEEN | |
| | FKALVLIAFAQYLQQCPFEDHVKLVNEVTEFAKTCVADESAENCD | |
| | KSLHTLFGDKLCTVATLRETYGEMADCCAKQEPERNECFLQHKDD | |
| | NPNLPRLVRPEVDVMCTAFHDNEETFLKKYLYEIARRHPYFYAPE | |
| | LLFFAKRYKAAFTECCQAADKAACLLPKLDELRDEGKASSAKQRL | |
| | KCASLQKFGERAFKAWAVARLSQRFPKAEFAEVSKLVTDLTKVHT | |
| | ECCHGDLLECADDRADLAKYICENQDSISSKLKECCEKPLLEKSH | |
| | CIAEVENDEMPADLPSLAADFVESKDVCKNYAEAKDVFLGMFLYE | |
| | YARRHPDYSVVLLLRLAKTYETTLEKCCAAADPHECYAKVFDEFK | |
| | PLVEEPQNLIKQNCELFEQLGEYKFQNALLVRYTKKVPQVSTPTL | |
| | VEVSRNLGKVGSKCCKHPEAKRMPCAEDYLSVVLNQLCVLHEKTP | |
| | VSDRVTKCCTESLVNRRPCFSALEVDETYVPKEFNAETFTFHADI | |
| | CTLSEKERQIKKQTALVELVKHKPKATKEQLKAVMDDFAAFVEKC | |
| | CKADDKETCFAEEGKKLVAASQAALGL | |

TABLE 2
Primers used in construction of human Lefty A
fusion proteins

| | | SEQ |
|---|---|---|
| Primer | | ID |
| names | Sequences((5′→3′) | NOS: |
| L1_F | AATTAAGCTTGCCACCATGTGGCCTCTGTGG | 13 |
| | CTCTG | |
| L2_R | AGGTTGCAATCGTCTTGGGACAAG | 14 |
| L3_F | CTTGTCCCAAGACGATTGCAACCTGAGCCCA | 15 |
| | AATCTTGTGACAAA | |
| L4_R | AATTCTCGAGTCATTTACCCGGAGACAGGGA | 16 |
| L5_F | GTCCCAAGACGATTGCAACCTAGCGGCGGTG | 17 |
| | GCGGTTCTGGCGGTGGTGGAAGTGGCGGTGG | |
| | CGGGTCTGAGCCCAAATCTTGTGAC | |
| L6_F | CTCTCCCTGTCTCCGGGTAAACTCACCGAAG | 18 |
| | AGCAGTTGTTG | |
| L7_F | CTCTCCCTGTCTCCGGGTAAATTCAGCCAAT | 19 |
| | CATTCCGTGAG | |
| L8_F | CTCTCCCTGTCTCCGGGTAAACTCTCCCCCC | 20 |
| | GAAGCGCCCAG | |
| L9_R | GAAGGCACAGCTCGAGTCAAGGTTGCAATCG | 21 |
| | TCTTGGGACAAG | |
| L10_F | GTCCCAAGACGATTGCAACCTGACGCTCACA | 22 |
| | AGTCTGAAGTTGCT | |
| L11_R | AGATCCACGCGGAACCAGCTCGAGTCACAGC | 23 |
| | CCCAAAGCGGCCTGTGA | |

### Example 1-6: Transient Expression, Purification and Analysis of Human Lefty A Fusion Proteins

Human Lefty A and its variant proteins were expressed using the Freestyle™ MAX CHO Expression system. Specifically, Freestyle™ CHO—S cells (Invitrogen, USA) were transfected with the expression vectors described above and were grown in CHO serum-free medium (Invitrogen, USA) for 5 days. The culture medium was centrifuged, and the supernatant was recovered, filtered, and then used later in the protein expression level analysis and purification of Lefty A in the culture medium.

The Fc fusion protein from the culture was purified by Protein A-based affinity chromatography using MabSelect Protein A resin (GE Healthcare, USA). A column packed with Protein A resin was equilibrated with phosphate-buffered saline (PBS, pH 7.4), and then the filtered cell culture supernatant was loaded on the column. After washing with 10 column volumes (CV) of PBS, and then the protein was eluted with 5 CV of elution buffer (0.1M Sodium Citrate), and the eluate was neutralized by adding 200 μl of 1M Tris-HCl (pH 8.0). To buffer-exchange the recombinant protein-eluted fraction with PBS (pH 7.4), the Amicon Ultra-15 Centrifugal filter (MWCO: 30000) (Millipore, Cat. No. UFC903096) was used. The purified protein fraction pooling sample was placed, centrifuged at 4,000 rpm and 4° C. for 10 minutes, diluted 10-fold, and then centrifuged three times, so that it was buffer-exchanged 1,000-fold or more. The purified recombinant protein was filtered through a 25 mm PES syringe filter (0.22 μm; Nalgene, Cat. No. NAL-194-2520) in a clean bench while minimizing the loss of the sample, and then sealed and stored at 4° C. without exposure to the outside.

The HSA fusion protein was purified using the CaptureSelect™ HSA affinity matrix (ThermoFisher Scientific, USA) or HiTrap Blue HP resin that binds specifically to HSA. The filtered culture supernatant was passed through the PBS-packed CaptureSelect affinity column, washed with 20 mM Tris-Cl containing 1M NaCl, and then eluted using 20 mM Tris-Cl containing 1M arginine and 1M NaCl. Alternatively, the prepared culture supernatant was passed through the HiTrap Blue HP column packed with 20 mM sodium phosphate (pH 7.0) as a binding buffer so that only the desired protein was bound to the resin, and then the protein was eluted using 20 mM sodium phosphate (pH 7.0) using 2M NaCl. After completion of the purification, the protein concentration of each fraction was measured at a wavelength of 280 nm with the NANODROP 2000 spectrophotometer (Thermo Scientific, Cat. No. ND-2000). In order to measure whether each fraction would contain the desired protein, 1200 series HPLC (Agilent Technologies) with SWxl Guard column (TOSOH, Cat. No. 08543) and TSKgel G3000SWxl (TOSOH, Cat. No. 08541) was used as size exclusion HPLC.

Each of the purified fusion proteins was analyzed by SDS-PAGE and size exclusion chromatography (SEC). SDS-PAGE was performed on Tris/glycine 4-12% acrylamide gel (Invitrogen, USA). The gel for analysis was stained with Coomassie blue and imaged by digital scanning. Each of the purified proteins was analyzed by size exclusion chromatography (SEC) using a TSK-GEL G300 SWXL column (7.8×300 mm, Tosohaas, USA), equilibrated in phosphate buffered saline containing 0.02% NaN₃, at a flow rate of 0.5 ml/min, thereby determining the expression level and purity of the protein. The results are shown in Table 3 below.

TABLE 3
Purification yield and purity of human Lefty
A fusion proteins

| Protein names | Yield (μg/L) | Purity SEC (%) |
|---|---|---|
| 42 Fc | 63.8 | 45.9 |
| 34 Fc | not expressed | — |
| 28 Fc | not expressed | — |
| 42 LFc | 125 | 46.2 |
| 34 LFc | not expressed | — |
| 28 LFc | not expressed | — |
| Fc 42 | 7.2 | — |
| Fc 34 | not expressed | — |
| Fc 28 | not expressed | — |

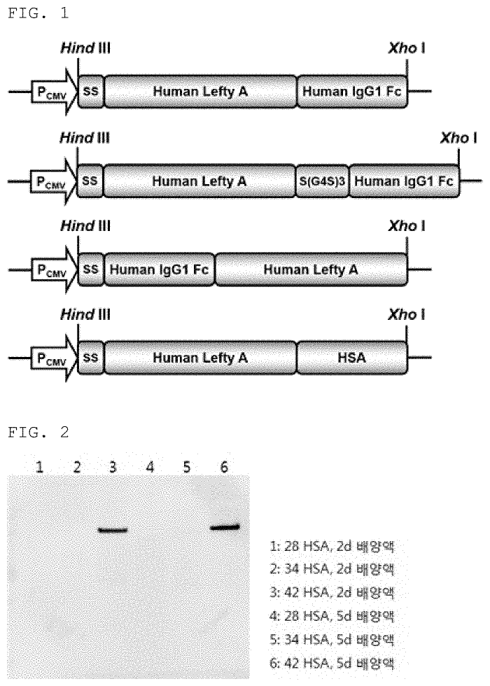

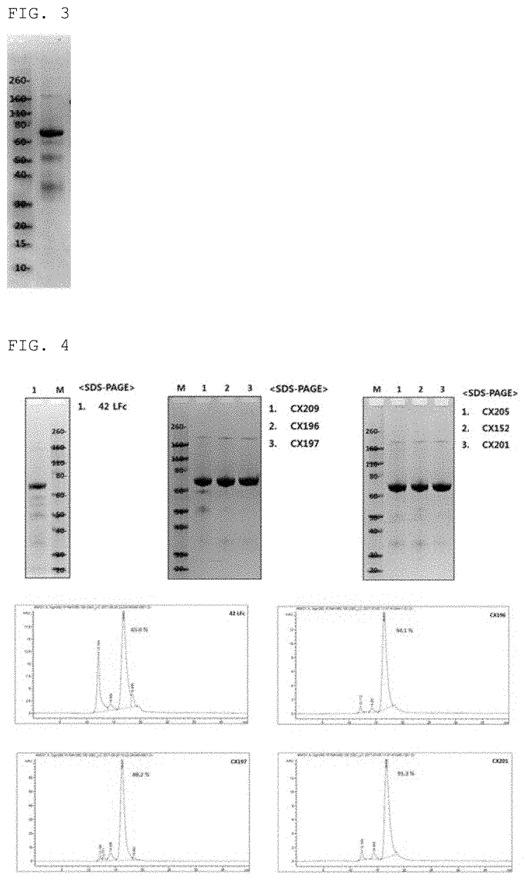

The 34 or 28 Fc fusion protein was not expressed regardless of the Fc fusion site. In the case of the “42” fragments, when the protein expression efficiency of the C-terminal fusion (“42 Fc”) significantly increased compared to that of the N-terminal fusion (“Fc 42”), and particularly, in the case of the linker Fc fusion (“42 LFc”), the protein expression efficiency greatly increased. Also, in the case of the HSA fusion proteins, only 42 HSA was expressed, and neither 34 HSA nor 28 HSA was expressed. This was also confirmed by Western blot analysis of the culture (FIG. 2).

Based on these expression data and in vivo efficacy data described in Example 6, 42 Fc or 42 LFc was used later as a template in the construction of variants.

### Example 2: Construction of Human Lefty A Fusion Protein Variants with Improved Productivity and Stability

Based on the expression vector described in Example 1, Lefty A fusion protein variants were constructed with improved protein stability and expression in animal cells compared to 42 LFc.

### Example 2-1: Construction of Human Lefty A Fusion Protein Variant Expression Vectors

The propeptide-removed fusion proteins constructed in Example 1 were mostly not expressed. Even the 42 LFc protein was poorly expressed in the host cells and the purified protein had poor purity and lower yield. As shown in FIG. 3, after affinity purification of 42 LFc proteins, additional smaller bands appeared on a SDS-PAGE gel, which may be caused by cleavage at the processing sites or non-specific cleavage due to protein instability (FIG. 3). In addition, potential cleavage sites cleaved by various enzymes and chemical substances in human Lefty A sequence were predicted using

ExPASy PeptideCutter DB (https://web.expasy.org/peptide cutter/), and as a result, it was predicted that the leucine residues at positions 311 and 359 would be cleaved by thrombin enzyme. Thus, a mutation was introduced into the propeptide domain, the processing sites and positions L311, P313, R314, L359, P361 and R362 in order to increase the expression level, stability and purification purity of the human Lefty A fusion protein variants. Using the 42 LFc gene as a template and the primer pairs shown in Table 4 below, the respective fragments were obtained by PCR under the same conditions as described in Example 1, and then subjected to an assembly PCR reaction. Each of the obtained reaction products was separated and purified by 1.5% agarose gel electrophoresis, and then ligated with a pCLSO5 vector, digested with the restriction enzymes Hind III and Xho I, using the In-Fusion® HD Cloning Kit (Clontech, 639650) at 50° C. for 15 minutes. The subsequent procedure was the same as described above with respect to the construction of the C-terminal Fc fusion proteins.

TABLE 4
Primers used in construction of
human Lefty A variants

| Primer | | SEQ ID |
|---|---|---|
| names | Sequences (5′→3′) | NOS: |
| L12_F | ACTCTAGAGGATCGAACCCTT | 24 |
| L12_R | GCTGGCAACTAGAAGGCACAGCTCGAGTCATTT | 25 |
| | ACCCGGAGACAGGGAGAGGCT | |
| L13_F | GGGGCCGCTCTCACAGRGGAGCAGKTGCTCGGG | 26 |
| | TCATTGTTG | |
| L13_R | CAACAATGACCCGAGCAMCTGCTCCYCTGTGAG | 27 |
| | AGCGGCCCC | |
| L14_F | CTCGGGTCATTGTTGAGGCAGCTTCAGCTTTCC | 28 |
| L14_R | GGAAAGCTGAAGCTGCCTCAACAATGAGCCGAG | 29 |
| L15_F | CTTCAGCTTTCCGAAGTGCCCGTCCTGGACAAG | 30 |
| L15_R | CTTGTCCAGGACGGGCACTTCGGAAAGCTGAAG | 31 |
| L16_F | CCACCCGTCCTGGACARGGCCGATRTGGAGGGG | 32 |
| | CTCGTTATC | |
| L16_R | GATAACGAGCCCCTCCAYATCGGCCYTGTCCAG | 33 |
| | GACGGGTGG | |
| L17_F | AAGGCCGATGTCGAGAAGCTCGTTATCCCATCC | 34 |
| L17_R | GGATGGGATAACGAGCTTCTCGACATCGGCCTT | 35 |
| L18_F | GGGCTCGTTATCCCAGCCCATGTCCGGGCTCAG | 36 |
| L18_R | CTGAGCCCGGACATGGGCTGGGATAACGAGCCC | 37 |
| L19_F | CGGGCTCAGTACGTTGTGTTGCTGCAACATTCA | 38 |
| L19_R | TGAATGTTGGAGCAACACAACGTACTGAGCCCG | 39 |
| L20_F | TACGTTGCCTTGCTGAGACGGTCACACGCTAGT | 40 |
| | AGA | |
| L20_R | TCTACTAGCGTGTGACCGTCTCAGCAAGGCAAC | 41 |
| | GTA | |
| L21_F | CTGCAACATTCACACGSAKMCAGAAGTGGAGGC | 42 |
| | AAG | |
| L21_R | CTTGCCTCCACTTCTGKMTSCGTGTGAATGTTG | 43 |
| | CAG | |
| L22_F | TACGTTGCCTTGCTGCRGCRCTCACACGCTAGT | 44 |
| | AGA | |
| L22_R | TCTACTAGCGTGTGAGYGCYGCAGCAAGGCAAC | 45 |
| | GTA | |
| L23_F | GGGGCCGCCCTCACCGGCGAGCAGTTGTTGGGC | 46 |
| L23_R | GCCCAACAACTGCTCGCCGGTGAGGGCGGCCCC | 47 |
| L24_F | CGCGCCCAATATGTGGYCCTGCTCCRGCGATCT | 48 |
| | CACGGAGAT | |
| L24_R | ATCTCCGTGAGATCGCYGGAGCAGGRCCACATA | 49 |
| | TTGGGCGCG | |
| L25_F | CGCGCCCAATATGTGGCCCTGCTCCRGCACTCT | 50 |
| | CACGGAGATAGG | |
| L25_R | CCTATCTCCGTGAGAGTGCYGGAGCAGGGCCAC | 51 |
| | ATATTGGGCGCG | |
| L26_F | AAGGCCGATGTCGAGAAGCTCGTTATCCCAGCC | 52 |
| | CATGTCCGGGCTCAG | |
| L26_R | CTGAGCCCGGACATGGGCTGGGATAACGAGCTT | 53 |
| | CTCGACATCGGCCTT | |
| L27_F | AAGGCTGCCCTGCACCGGCATGGTCGTCTCTCC | 54 |
| | CCCCGAAGC | |
| L27_R | GCTTCGGGGGGAGAGACGACCATGCCGGTGCAG | 55 |
| | GGCAGCCTT | |
| L28_F | CTGCACGGCCATGGTGGCCTCTCCCCCCGAAGC | 56 |
| L28_R | GCTTCGGGGGGAGAGGCCACCATGGCCGTGCAG | 57 |
| L29_F | AGGAGCAGGGGCAAGGYCTTCAGCCAATCATTC | 58 |
| L29_R | GAATGATTGGCTGAAGRCCTTGCCCCTGCTCCT | 59 |
| L30_F | CTGCACCGGCATGGTGYCCTCTCCCCCCGAAGC | 60 |
| L30_R | GCTTCGGGGGGAGAGGRCACCATGCCGGTGCAG | 61 |
| L31_F | CTGCACCGGCATGGTGGCCTCTCCCCCCGAAGC | 62 |
| L31_R | GCTTCGGGGGGAGAGGCCACCATGCCGGTGCAG | 63 |
| L32_F | CTCCTTCTGCAAGTGNNKGTCCAGCGCGAACAC | 64 |
| L32_R | GTGTTCGCGCTGGACMNNCACTTGCAGAAGGAG | 65 |
| L33_F | CTGGTGCGATTCGCTNNKCAGGGTGCACCAGCC | 66 |
| L33_R | GGCTGGTGCACCCTGMNNAGCGAATCGCACCAG | 67 |
| L34_F | TTCAACTGGCCCTTTGASGGACCTCGGCAATGT | 68 |
| | ATTGCCAGC | |
| L34_R | GCTGGCAATACATTGCCGAGGTCCSTCAAAGGG | 69 |
| | CCAGTTGAA | |
| L35_F | TTCAACTGGCCCTTTCTCGGAGASCGGCAATGT | 70 |
| | ATTGCCAGC | |
| L35_R | GCTGGCAATACATTGCCGSTCTCCGAGAAAGGG | 71 |
| | CCAGTTGAA | |
| L36_F | TTCAACTGGCCCTTTCTCGGATCCCGGCAATGT | 72 |
| | ATTGCCAGC | |
| L36_R | GCTGGCAATACATTGCCGGGATCCGAGAAAGGG | 73 |
| | CCAGTTGAA | |
| L37_F | TTCAACTGGCCCTTTCTCGGACCTMAGCAATGT | 74 |
| | ATTGCCAGC | |
| L37_R | GCTGGCAATACATTGCTKAGGTCCGAGAAAGGG | 75 |
| | CCAGTTGAA | |
| L38_F | GCCTCTGACGGGGCTGASGTCCCAAGACGATTG | 76 |
| | CAACCTTCT | |
| L38_R | AGAAGGTTGCAATCGTCTTGGGACSTCAGCCCC | 77 |
| | GTCAGAGGC | |
| L39_F | GCCTCTGACGGGGCTCTTGTCGASAGACGATTG | 78 |
| | CAACCTTCT | |
| L39_R | AGAAGGTTGCAATCGTCTSTCGACAAGAGCCCC | 79 |
| | GTCAGAGGC | |
| L40_F | GCCTCTGACGGGGCTCTTGTCTCCAGACGATTG | 80 |
| | CAACCTTCT | |
| L40_R | AGAAGGTTGCAATCGTCTGGAGACAAGAGCCCC | 81 |
| | GTCAGAGGC | |
| L41_F | GCCTCTGACGGGGCTCTTGTCCCAMAGCGATTG | 82 |
| | CAACCTTCT | |
| L41_R | AGAAGGTTGCAATCGCTKTGGGACAAGAGCCCC | 83 |
| | GTCAGAGGC | |
| L42_F | GCCTCTGACGGGGCTCTTGTCGACCAGCGATTG | 84 |
| | CAACCTTCT | |
| L42_R | AGAAGGTTGCAATCGCTGGTCGACAAGAGCCCC | 85 |
| | GTCAGAGGC | |

The amino acid sequences of a variant in which a substitution of amino acid residues in the propeptide domain (L22 to S73) in the sequence of 42 LFc occurred are shown in

Table 5 below.

TABLE 5
Amino acid sequences of human Lefty A fusion
protein variant (propeptide domain variant)

| | | SEQ |
|---|---|---|
| | | ID |
| | Amino acid sequences | NOS: |
| 42 LFc | MWPLWLCWALWVLPLAGPGAALTEEQLLGSLLRQLQLSEVPVLD | 86 |
| (V63A) | RADMEKLVIPAHVRAQYVALLRRSHGDRSGGKGFSQSFREVAGR | |
| | FLASEASTHLLVFGMEQRLPPNSELVQAVLRLFQEPVPKAALHG | |
| | HGRLSPRSAQARVTVEWLRVRDDGSNRTSLIDSRLVSVHESGWK | |
| | AFDVTEAVNFWQQLSRPRQPLLLQVSVQREHLGPLASGAHKLVR | |
| | FASQGAPAGLGEPQLELHTLDLRDYGAQGDCDPEAPMTEGTRCC | |
| | RQEMYIDLQGMKWAKNWVLEPPGFLAYECVGTCQQPPEALAFNW | |
| | PFLGPRQCIASETASLPMIVSIKEGGRTRPQVVSLPNMRVQKCS | |
| | CASDGALVPRRLQPSGGGGSGGGGSGGGGSEPKSCDKTHTCPPC | |
| | PAPELLGGPSVFLFPPKPKDTLMISRTPEVTCVVVDVSHEDPEV | |
| | KFNWYVDGVEVHNAKTKPREEQYNSTYRVVSVLTVLHQDWLNGK | |
| | EYKCKVSNKALPAPIEKTISKAKGQPREPQVYTLPPSRDELTKN | |
| | QVSLTCLVKGFYPSDIAVEWESNGQPENNYKTTPPVLDSDGSFF | |
| | LYSKLTVDKSRWQQGNVFSCSVMHEALHNHYTQKSLSLSPGK | |
| 42 LFc | MWPLWLCWALWVLPLAGPGAALTEEQLLGSLLRQLQLSEVPVLD | 87 |
| (R66Q) | RADMEKLVIPAHVRAQYVVLLQRSHGDRSGGKGFSQSFREVAGR | |
| | FLASEASTHLLVFGMEQRLPPNSELVQAVLRLFQEPVPKAALHG | |
| | HGRLSPRSAQARVTVEWLRVRDDGSNRTSLIDSRLVSVHESGWK | |
| | AFDVTEAVNFWQQLSRPRQPLLLQVSVQREHLGPLASGAHKLVR | |
| | FASQGAPAGLGEPQLELHTLDLRDYGAQGDCDPEAPMTEGTRCC | |
| | RQEMYIDLQGMKWAKNWVLEPPGFLAYECVGTCQQPPEALAFNW | |
| | PFLGPRQCIASETASLPMIVSIKEGGRTRPQVVSLPNMRVQKCS | |
| | CASDGALVPRRLQPSGGGGSGGGGSGGGGSEPKSCDKTHTCPPC | |
| | PAPELLGGPSVFLFPPKPKDTLMISRTPEVTCVVVDVSHEDPEV | |
| | KFNWYVDGVEVHNAKTKPREEQYNSTYRVVSVLTVLHQDWLNGK | |
| | EYKCKVSNKALPAPIEKTISKAKGQPREPQVYTLPPSRDELTKN | |
| | QVSLTCLVKGFYPSDIAVEWESNGQPENNYKTTPPVLDSDGSFF | |
| | LYSKLTVDKSRWQQGNVFSCSVMHEALHNHYTQKSLSLSPGK | |
| 42 LFc | MWPLWLCWALWVLPLAGPGAALTEEQLLGSLLRQLQLSEVPVLD | 88 |
| (V63A/ | RADMEKLVIPAHVRAQYVALLQRSHGDRSGGKGFSQSFREVAGR | |
| R66Q) | FLASEASTHLLVFGMEQRLPPNSELVQAVLRLFQEPVPKAALHG | |
| | HGRLSPRSAQARVTVEWLRVRDDGSNRTSLIDSRLVSVHESGWK | |
| | AFDVTEAVNFWQQLSRPRQPLLLQVSVQREHLGPLASGAHKLVR | |
| | FASQGAPAGLGEPQLELHTLDLRDYGAQGDCDPEAPMTEGTRCC | |
| | RQEMYIDLQGMKWAKNWVLEPPGFLAYECVGTCQQPPEALAFNW | |
| | PFLGPRQCIASETASLPMIVSIKEGGRTRPQVVSLPNMRVQKCS | |
| | CASDGALVPRRLQPSGGGGSGGGGSGGGGSEPKSCDKTHTCPPC | |
| | PAPELLGGPSVFLFPPKPKDTLMISRTPEVTCVVVDVSHEDPEV | |
| | KFNWYVDGVEVHNAKTKPREEQYNSTYRVVSVLTVLHQDWLNGK | |
| | EYKCKVSNKALPAPIEKTISKAKGQPREPQVYTLPPSRDELTKN | |
| | QVSLTCLVKGFYPSDIAVEWESNGQPENNYKTTPPVLDSDGSFF | |
| | LYSKLTVDKSRWQQGNVFSCSVMHEALHNHYTQKSLSLSPGK | |
| 42 LFc | MWPLWLCWALWVLPLAGPGAALTEEQLLGSLLRQLQLSEVPVLD | 89 |
| (V63A/ | RADMEKLVIPAHVRAQYVALLRHSHGDRSGGKGFSQSFREVAGR | |
| R67H) | FLASEASTHLLVFGMEQRLPPNSELVQAVLRLFQEPVPKAALHG | |
| | HGRLSPRSAQARVTVEWLRVRDDGSNRTSLIDSRLVSVHESGWK | |
| | AFDVTEAVNFWQQLSRPRQPLLLQVSVQREHLGPLASGAHKLVR | |
| | FASQGAPAGLGEPQLELHTLDLRDYGAQGDCDPEAPMTEGTRCC | |
| | RQEMYIDLQGMKWAKNWVLEPPGFLAYECVGTCQQPPEALAFNW | |
| | PFLGPRQCIASETASLPMIVSIKEGGRTRPQVVSLPNMRVQKCS | |
| | CASDGALVPRRLQPSGGGGSGGGGSGGGGSEPKSCDKTHTCPPC | |
| | PAPELLGGPSVFLFPPKPKDTLMISRTPEVTCVVVDVSHEDPEV | |
| | KFNWYVDGVEVHNAKTKPREEQYNSTYRVVSVLTVLHQDWLNGK | |
| | EYKCKVSNKALPAPIEKTISKAKGQPREPQVYTLPPSRDELTKN | |
| | QVSLTCLVKGFYPSDIAVEWESNGQPENNYKTTPPVLDSDGSFF | |
| | LYSKLTVDKSRWQQGNVFSCSVMHEALHNHYTQKSLSLSPGK | |
| 42 LFc | MWPLWLCWALWVLPLAGPGAALTEEQLLGSLLRQLQLSEVPVLD | 90 |
| (V63A/ | RADMEKLVIPAHVRAQYVALLQHSHGDRSGGKGFSQSFREVAGR | |
| R66Q/ | FLASEASTHLLVFGMEQRLPPNSELVQAVLRLFQEPVPKAALHG | |
| R67H) | HGRLSPRSAQARVTVEWLRVRDDGSNRTSLIDSRLVSVHESGWK | |
| | AFDVTEAVNFWQQLSRPRQPLLLQVSVQREHLGPLASGAHKLVR | |
| | FASQGAPAGLGEPQLELHTLDLRDYGAQGDCDPEAPMTEGTRCC | |
| | RQEMYIDLQGMKWAKNWVLEPPGFLAYECVGTCQQPPEALAFNW | |
| | PFLGPRQCIASETASLPMIVSIKEGGRTRPQVVSLPNMRVQKCS | |
| | CASDGALVPRRLQPSGGGGSGGGGSGGGGSEPKSCDKTHTCPPC | |
| | PAPELLGGPSVFLFPPKPKDTLMISRTPEVTCVVVDVSHEDPEV | |
| | KFNWYVDGVEVHNAKTKPREEQYNSTYRVVSVLTVLHQDWLNGK | |
| | EYKCKVSNKALPAPIEKTISKAKGQPREPQVYTLPPSRDELTKN | |
| | QVSLTCLVKGFYPSDIAVEWESNGQPENNYKTTPPVLDSDGSFF | |
| | LYSKLTVDKSRWQQGNVFSCSVMHEALHNHYTQKSLSLSPGK | |
| LFB 42 | MWPLWLCWALWVLPLAGPGAALTGEQLLGSLLRQLQLKEVPTLD | 91 |
| LFc | RADMEELVIPTHVRAQYVALLQRSHGDRSGGKGFSQSFREVAGR | |
| | FLASEASTHLLVFGMEQRLPPNSELVQAVLRLFQEPVPKAALHG | |
| | HGRLSPRSAQARVTVEWLRVRDDGSNRTSLIDSRLVSVHESGWK | |
| | AFDVTEAVNFWQQLSRPRQPLLLQVSVQREHLGPLASGAHKLVR | |
| | FASQGAPAGLGEPQLELHTLDLRDYGAQGDCDPEAPMTEGTRCC | |
| | RQEMYIDLQGMKWAKNWVLEPPGFLAYECVGTCQQPPEALAFNW | |
| | PFLGPRQCIASETASLPMIVSIKEGGRTRPQVVSLPNMRVQKCS | |
| | CASDGALVPRRLQPSGGGGSGGGGSGGGGSEPKSCDKTHTCPPC | |
| | PAPELLGGPSVFLFPPKPKDTLMISRTPEVTCVVVDVSHEDPEV | |
| | KFNWYVDGVEVHNAKTKPREEQYNSTYRVVSVLTVLHQDWLNGK | |
| | EYKCKVSNKALPAPIEKTISKAKGQPREPQVYTLPPSRDELTKN | |
| | QVSLTCLVKGFYPSDIAVEWESNGQPENNYKTTPPVLDSDGSFF | |
| | LYSKLTVDKSRWQQGNVFSCSVMHEALHNHYTQKSLSLSPGK | |

The LFB 42 LFc is a variant in which amino acid residue substitutions of E24G, S38K, V42T, K50E, A55T, V63A and R66Q in the sequence of 42 LFc occurred.

The amino acid sequences of a variant in which a substitution of amino acid residues at the processing sites (R74 to R77 and R132 to R135) in the sequence of 42 LFc occurred are shown in Table 6 below.

TABLE 6
Amino acid sequences of human Lefty A fusion
protein variants (processing site variants)

| | | SEQ |
|---|---|---|
| | | ID |
| | Amino acid sequences | NOS: |
| 42 LFc | MWPLWLCWALWVLPLAGPGAALTEEQLLGSLLRQLQLSEVPVLDRA | 92 |
| V1 | DMEKLVIPAHVRAQYVVLLRRSHGDRSGGKGFSQSFREVAGRFLAS | |
| | EASTHLLVFGMEQRLPPNSELVQAVLRLFQEPVPKAALHRHGRLSP | |
| | RSAQARVTVEWLRVRDDGSNRTSLIDSRLVSVHESGWKAFDVTEAV | |
| | NFWQQLSRPRQPLLLQVSVQREHLGPLASGAHKLVRFASQGAPAGL | |
| | GEPQLELHTLDLRDYGAQGDCDPEAPMTEGTRCCRQEMYIDLQGMK | |
| | WAKNWVLEPPGFLAYECVGTCQQPPEALAFNWPFLGPRQCIASETA | |
| | SLPMIVSIKEGGRTRPQVVSLPNMRVQKCSCASDGALVPRRLQPSG | |
| | GGGSGGGGSGGGGSEPKSCDKTHTCPPCPAPELLGGPSVFLFPPKP | |
| | KDTLMISRTPEVTCVVVDVSHEDPEVKFNWYVDGVEVHNAKTKPRE | |
| | EQYNSTYRVVSVLTVLHQDWLNGKEYKCKVSNKALPAPIEKTISKA | |
| | KGQPREPQVYTLPPSRDELTKNQVSLTCLVKGFYPSDIAVEWESNG | |
| | QPENNYKTTPPVLDSDGSFFLYSKLTVDKSRWQQGNVFSCSVMHEA | |
| | LHNHYTQKSLSLSPGK | |
| 42 LFc | MWPLWLCWALWVLPLAGPGAALTEEQLLGSLLRQLQLSEVPVLDRA | 93 |
| V2 | DMEKLVIPAHVRAQYVVLLRRSHGDRSGGKGFSQSFREVAGRFLAS | |
| | EASTHLLVFGMEQRLPPNSELVQAVLRLFQEPVPKAALHRHGGLSP | |
| | RSAQARVTVEWLRVRDDGSNRTSLIDSRLVSVHESGWKAFDVTEAV | |
| | NFWQQLSRPRQPLLLQVSVQREHLGPLASGAHKLVRFASQGAPAGL | |
| | GEPQLELHTLDLRDYGAQGDCDPEAPMTEGTRCCRQEMYIDLQGMK | |
| | WAKNWVLEPPGFLAYECVGTCQQPPEALAFNWPFLGPRQCIASETA | |
| | SLPMIVSIKEGGRTRPQVVSLPNMRVQKCSCASDGALVPRRLQPSG | |
| | GGGSGGGGSGGGGSEPKSCDKTHTCPPCPAPELLGGPSVFLFPPKP | |
| | KDTLMISRTPEVTCVVVDVSHEDPEVKFNWYVDGVEVHNAKTKPRE | |
| | EQYNSTYRVVSVLTVLHQDWLNGKEYKCKVSNKALPAPIEKTISKA | |
| | KGQPREPQVYTLPPSRDELTKNQVSLTCLVKGFYPSDIAVEWESNG | |
| | QPENNYKTTPPVLDSDGSFFLYSKLTVDKSRWQQGNVFSCSVMHEA | |
| | LHNHYTQKSLSLSPGK | |
| 42 LFc | MWPLWLCWALWVLPLAGPGAALTEEQLLGSLLRQLQLSEVPVLDRA | 94 |
| V3 | DMEKLVIPAHVRAQYVVLLRRSHGDRSGGKGFSQSFREVAGRFLAS | |
| | EASTHLLVFGMEQRLPPNSELVQAVLRLFQEPVPKAALHRHERLSP | |
| | RSAQARVTVEWLRVRDDGSNRTSLIDSRLVSVHESGWKAFDVTEAV | |
| | NFWQQLSRPRQPLLLQVSVQREHLGPLASGAHKLVRFASQGAPAGL | |
| | GEPQLELHTLDLRDYGAQGDCDPEAPMTEGTRCCRQEMYIDLQGMK | |
| | WAKNWVLEPPGFLAYECVGTCQQPPEALAFNWPFLGPRQCIASETA | |
| | SLPMIVSIKEGGRTRPQVVSLPNMRVQKCSCASDGALVPRRLQPSG | |
| | GGGSGGGGSGGGGSEPKSCDKTHTCPPCPAPELLGGPSVFLFPPKP | |
| | KDTLMISRTPEVTCVVVDVSHEDPEVKFNWYVDGVEVHNAKTKPRE | |
| | EQYNSTYRVVSVLTVLHQDWLNGKEYKCKVSNKALPAPIEKTISKA | |
| | KGQPREPQVYTLPPSRDELTKNQVSLTCLVKGFYPSDIAVEWESNG | |
| | QPENNYKTTPPVLDSDGSFFLYSKLTVDKSRWQQGNVFSCSVMHEA | |
| | LHNHYTQKSLSLSPGK | |
| 42 LFc | MWPLWLCWALWVLPLAGPGAALTEEQLLGSLLRQLQLSEVPVLDRA | 95 |
| V4 | DMEKLVIPAHVRAQYVVLLRRSHGDRSGGKGFSQSFREVAGRFLAS | |
| | EASTHLLVFGMEQRLPPNSELVQAVLRLFQEPVPKAALHGHGGLSP | |
| | RSAQARVTVEWLRVRDDGSNRTSLIDSRLVSVHESGWKAFDVTEAV | |
| | NFWQQLSRPRQPLLLQVSVQREHLGPLASGAHKLVRFASQGAPAGL | |
| | GEPQLELHTLDLRDYGAQGDCDPEAPMTEGTRCCRQEMYIDLQGMK | |
| | WAKNWVLEPPGFLAYECVGTCQQPPEALAFNWPFLGPRQCIASETA | |
| | SLPMIVSIKEGGRTRPQVVSLPNMRVQKCSCASDGALVPRRLQPSG | |
| | GGGSGGGGSGGGGSEPKSCDKTHTCPPCPAPELLGGPSVFLFPPKP | |
| | KDTLMISRTPEVTCVVVDVSHEDPEVKFNWYVDGVEVHNAKTKPRE | |
| | EQYNSTYRVVSVLTVLHQDWLNGKEYKCKVSNKALPAPIEKTISKA | |
| | KGQPREPQVYTLPPSRDELTKNQVSLTCLVKGFYPSDIAVEWESNG | |
| | QPENNYKTTPPVLDSDGSFFLYSKLTVDKSRWQQGNVFSCSVMHEA | |
| | LHNHYTQKSLSLSPGK | |
| 42 LFc | MWPLWLCWALWVLPLAGPGAALTEEQLLGSLLRQLQLSEVPVLDRA | 96 |
| V5 | DMEKLVIPAHVRAQYVVLLRRSHGDRSRGKAFSQSFREVAGRFLAS | |
| | EASTHLLVFGMEQRLPPNSELVQAVLRLFQEPVPKAALHGHGRLSP | |
| | RSAQARVTVEWLRVRDDGSNRTSLIDSRLVSVHESGWKAFDVTEAV | |
| | NFWQQLSRPRQPLLLQVSVQREHLGPLASGAHKLVRFASQGAPAGL | |
| | GEPQLELHTLDLRDYGAQGDCDPEAPMTEGTRCCRQEMYIDLQGMK | |
| | WAKNWVLEPPGFLAYECVGTCQQPPEALAFNWPFLGPRQCIASETA | |
| | SLPMIVSIKEGGRTRPQVVSLPNMRVQKCSCASDGALVPRRLQPSG | |
| | GGGSGGGGSGGGGSEPKSCDKTHTCPPCPAPELLGGPSVFLFPPKP | |
| | KDTLMISRTPEVTCVVVDVSHEDPEVKFNWYVDGVEVHNAKTKPRE | |
| | EQYNSTYRVVSVLTVLHQDWLNGKEYKCKVSNKALPAPIEKTISKA | |
| | KGQPREPQVYTLPPSRDELTKNQVSLTCLVKGFYPSDIAVEWESNG | |
| | QPENNYKTTPPVLDSDGSFFLYSKLTVDKSRWQQGNVFSCSVMHEA | |
| | LHNHYTQKSLSLSPGK | |
| 42 LFc | MWPLWLCWALWVLPLAGPGAALTEEQLLGSLLRQLQLSEVPVLDRA | 97 |
| V6 | DMEKLVIPAHVRAQYVVLLRRSHGDRSRGKAFSQSFREVAGRFLAS | |
| | EASTHLLVFGMEQRLPPNSELVQAVLRLFQEPVPKAALHRHGALSP | |
| | RSAQARVTVEWLRVRDDGSNRTSLIDSRLVSVHESGWKAFDVTEAV | |
| | NFWQQLSRPRQPLLLQVSVQREHLGPLASGAHKLVRFASQGAPAGL | |
| | GEPQLELHTLDLRDYGAQGDCDPEAPMTEGTRCCRQEMYIDLQGMK | |
| | WAKNWVLEPPGFLAYECVGTCQQPPEALAFNWPFLGPRQCIASETA | |
| | SLPMIVSIKEGGRTRPQVVSLPNMRVQKCSCASDGALVPRRLQPSG | |
| | GGGSGGGGSGGGGSEPKSCDKTHTCPPCPAPELLGGPSVFLFPPKP | |
| | KDTLMISRTPEVTCVVVDVSHEDPEVKFNWYVDGVEVHNAKTKPRE | |
| | EQYNSTYRVVSVLTVLHQDWLNGKEYKCKVSNKALPAPIEKTISKA | |
| | KGQPREPQVYTLPPSRDELTKNQVSLTCLVKGFYPSDIAVEWESNG | |
| | QPENNYKTTPPVLDSDGSFFLYSKLTVDKSRWQQGNVFSCSVMHEA | |
| | LHNHYTQKSLSLSPGK | |
| 42 LFc | MWPLWLCWALWVLPLAGPGAALTEEQLLGSLLRQLQLSEVPVLDRA | 98 |
| V7 | DMEKLVIPAHVRAQYVVLLRRSHGDRSRGKAFSQSFREVAGRFLAS | |
| | EASTHLLVFGMEQRLPPNSELVQAVLRLFQEPVPKAALHRHGVLSP | |
| | RSAQARVTVEWLRVRDDGSNRTSLIDSRLVSVHESGWKAFDVTEAV | |
| | NFWQQLSRPRQPLLLQVSVQREHLGPLASGAHKLVRFASQGAPAGL | |
| | GEPQLELHTLDLRDYGAQGDCDPEAPMTEGTRCCRQEMYIDLQGMK | |
| | WAKNWVLEPPGFLAYECVGTCQQPPEALAFNWPFLGPRQCIASETA | |
| | SLPMIVSIKEGGRTRPQVVSLPNMRVQKCSCASDGALVPRRLQPSG | |
| | GGGSGGGGSGGGGSEPKSCDKTHTCPPCPAPELLGGPSVFLFPPKP | |
| | KDTLMISRTPEVTCVVVDVSHEDPEVKFNWYVDGVEVHNAKTKPRE | |
| | EQYNSTYRVVSVLTVLHQDWLNGKEYKCKVSNKALPAPIEKTISKA | |
| | KGQPREPQVYTLPPSRDELTKNQVSLTCLVKGFYPSDIAVEWESNG | |
| | QPENNYKTTPPVLDSDGSFFLYSKLTVDKSRWQQGNVFSCSVMHEA | |
| | LHNHYTQKSLSLSPGK | |
| 42 LFc | MWPLWLCWALWVLPLAGPGAALTEEQLLGSLLRQLQLSEVPVLDRA | 99 |
| V8 | DMEKLVIPAHVRAQYVVLLRRSHGDRSRGKVFSQSFREVAGRFLAS | |
| | EASTHLLVFGMEQRLPPNSELVQAVLRLFQEPVPKAALHGHGRLSP | |
| | RSAQARVTVEWLRVRDDGSNRTSLIDSRLVSVHESGWKAFDVTEAV | |
| | NFWQQLSRPRQPLLLQVSVQREHLGPLASGAHKLVRFASQGAPAGL | |
| | GEPQLELHTLDLRDYGAQGDCDPEAPMTEGTRCCRQEMYIDLQGMK | |
| | WAKNWVLEPPGFLAYECVGTCQQPPEALAFNWPFLGPRQCIASETA | |
| | SLPMIVSIKEGGRTRPQVVSLPNMRVQKCSCASDGALVPRRLQPSG | |
| | GGGSGGGGSGGGGSEPKSCDKTHTCPPCPAPELLGGPSVFLFPPKP | |
| | KDTLMISRTPEVTCVVVDVSHEDPEVKFNWYVDGVEVHNAKTKPRE | |
| | EQYNSTYRVVSVLTVLHQDWLNGKEYKCKVSNKALPAPIEKTISKA | |
| | KGQPREPQVYTLPPSRDELTKNQVSLTCLVKGFYPSDIAVEWESNG | |
| | QPENNYKTTPPVLDSDGSFFLYSKLTVDKSRWQQGNVFSCSVMHEA | |
| | LHNHYTQKSLSLSPGK | |
| 42 LFc | MWPLWLCWALWVLPLAGPGAALTEEQLLGSLLRQLQLSEVPVLDRA | 100 |
| V9 | DMEKLVIPAHVRAQYVVLLRRSHGDRSRGKVFSQSFREVAGRFLAS | |
| | EASTHLLVFGMEQRLPPNSELVQAVLRLFQEPVPKAALHRHGALSP | |
| | RSAQARVTVEWLRVRDDGSNRTSLIDSRLVSVHESGWKAFDVTEAV | |
| | NFWQQLSRPRQPLLLQVSVQREHLGPLASGAHKLVRFASQGAPAGL | |
| | GEPQLELHTLDLRDYGAQGDCDPEAPMTEGTRCCRQEMYIDLQGMK | |
| | WAKNWVLEPPGFLAYECVGTCQQPPEALAFNWPFLGPRQCIASETA | |
| | SLPMIVSIKEGGRTRPQVVSLPNMRVQKCSCASDGALVPRRLQPSG | |
| | GGGSGGGGSGGGGSEPKSCDKTHTCPPCPAPELLGGPSVFLFPPKP | |
| | KDTLMISRTPEVTCVVVDVSHEDPEVKFNWYVDGVEVHNAKTKPRE | |
| | EQYNSTYRVVSVLTVLHQDWLNGKEYKCKVSNKALPAPIEKTISKA | |
| | KGQPREPQVYTLPPSRDELTKNQVSLTCLVKGFYPSDIAVEWESNG | |
| | QPENNYKTTPPVLDSDGSFFLYSKLTVDKSRWQQGNVFSCSVMHEA | |
| | LHNHYTQKSLSLSPGK | |
| 42 LFc | MWPLWLCWALWVLPLAGPGAALTEEQLLGSLLRQLQLSEVPVLDRA | 101 |
| V10 | DMEKLVIPAHVRAQYVVLLRRSHGDRSRGKVFSQSFREVAGRFLAS | |
| | EASTHLLVFGMEQRLPPNSELVQAVLRLFQEPVPKAALHRHGVLSP | |
| | RSAQARVTVEWLRVRDDGSNRTSLIDSRLVSVHESGWKAFDVTEAV | |
| | NFWQQLSRPRQPLLLQVSVQREHLGPLASGAHKLVRFASQGAPAGL | |
| | GEPQLELHTLDLRDYGAQGDCDPEAPMTEGTRCCRQEMYIDLQGMK | |
| | WAKNWVLEPPGFLAYECVGTCQQPPEALAFNWPFLGPRQCIASETA | |
| | SLPMIVSIKEGGRTRPQVVSLPNMRVQKCSCASDGALVPRRLQPSG | |
| | GGGSGGGGSGGGGSEPKSCDKTHTCPPCPAPELLGGPSVFLFPPKP | |
| | KDTLMISRTPEVTCVVVDVSHEDPEVKFNWYVDGVEVHNAKTKPRE | |
| | EQYNSTYRVVSVLTVLHQDWLNGKEYKCKVSNKALPAPIEKTISKA | |
| | KGQPREPQVYTLPPSRDELTKNQVSLTCLVKGFYPSDIAVEWESNG | |
| | QPENNYKTTPPVLDSDGSFFLYSKLTVDKSRWQQGNVFSCSVMHEA | |
| | LHNHYTQKSLSLSPGK | |
| 42 LFc | MWPLWLCWALWVLPLAGPGAALTEEQLLGSLLRQLQLSEVPVLDRA | 102 |
| V11 | DMEKLVIPAHVRAQYVVLLRRSHGDRSRHGGFSQSFREVAGRFLAS | |
| | EASTHLLVFGMEQRLPPNSELVQAVLRLFQEPVPKAALHRHGGLSP | |
| | RSAQARVTVEWLRVRDDGSNRTSLIDSRLVSVHESGWKAFDVTEAV | |
| | NFWQQLSRPRQPLLLQVSVQREHLGPLASGAHKLVRFASQGAPAGL | |
| | GEPQLELHTLDLRDYGAQGDCDPEAPMTEGTRCCRQEMYIDLQGMK | |
| | WAKNWVLEPPGFLAYECVGTCQQPPEALAFNWPFLGPRQCIASETA | |
| | SLPMIVSIKEGGRTRPQVVSLPNMRVQKCSCASDGALVPRRLQPSG | |
| | GGGSGGGGSGGGGSEPKSCDKTHTCPPCPAPELLGGPSVFLFPPKP | |
| | KDTLMISRTPEVTCVVVDVSHEDPEVKFNWYVDGVEVHNAKTKPRE | |
| | EQYNSTYRVVSVLTVLHQDWLNGKEYKCKVSNKALPAPIEKTISKA | |
| | KGQPREPQVYTLPPSRDELTKNQVSLTCLVKGFYPSDIAVEWESNG | |
| | QPENNYKTTPPVLDSDGSFFLYSKLTVDKSRWQQGNVFSCSVMHEA | |
| | LHNHYTQKSLSLSPGK | |
| 42 LFc | MWPLWLCWALWVLPLAGPGAALTEEQLLGSLLRQLQLSEVPVLDRA | 103 |
| V12 | DMEKLVIPAHVRAQYVVLLRRSHGDRSRGKGFSQSFREVAGRFLAS | |
| | EASTHLLVFGMEQRLPPNSELVQAVLRLFQEPVPKAALHRHGGLSP | |
| | RSAQARVTVEWLRVRDDGSNRTSLIDSRLVSVHESGWKAFDVTEAV | |
| | NFWQQLSRPRQPLLLQVSVQREHLGPLASGAHKLVRFASQGAPAGL | |
| | GEPQLELHTLDLRDYGAQGDCDPEAPMTEGTRCCRQEMYIDLQGMK | |
| | WAKNWVLEPPGFLAYECVGTCQQPPEALAFNWPFLGPRQCIASETA | |
| | SLPMIVSIKEGGRTRPQVVSLPNMRVQKCSCASDGALVPRRLQPSG | |
| | GGGSGGGGSGGGGSEPKSCDKTHTCPPCPAPELLGGPSVFLFPPKP | |
| | KDTLMISRTPEVTCVVVDVSHEDPEVKFNWYVDGVEVHNAKTKPRE | |
| | EQYNSTYRVVSVLTVLHQDWLNGKEYKCKVSNKALPAPIEKTISKA | |
| | KGQPREPQVYTLPPSRDELTKNQVSLTCLVKGFYPSDIAVEWESNG | |
| | QPENNYKTTPPVLDSDGSFFLYSKLTVDKSRWQQGNVFSCSVMHEA | |
| | LHNHYTQKSLSLSPGK | |
| 42 LFc | MWPLWLCWALWVLPLAGPGAALTEEQLLGSLLRQLQLSEVPVLDRA | 104 |
| V13 | DMEKLVIPAHVRAQYVVLLRRSHGDRSRGKAFSQSFREVAGRFLAS | |
| | EASTHLLVFGMEQRLPPNSELVQAVLRLFQEPVPKAALHRHGGLSP | |
| | RSAQARVTVEWLRVRDDGSNRTSLIDSRLVSVHESGWKAFDVTEAV | |
| | NFWQQLSRPRQPLLLQVSVQREHLGPLASGAHKLVRFASQGAPAGL | |
| | GEPQLELHTLDLRDYGAQGDCDPEAPMTEGTRCCRQEMYIDLQGMK | |
| | WAKNWVLEPPGFLAYECVGTCQQPPEALAFNWPFLGPRQCIASETA | |
| | SLPMIVSIKEGGRTRPQVVSLPNMRVQKCSCASDGALVPRRLQPSG | |
| | GGGSGGGGSGGGGSEPKSCDKTHTCPPCPAPELLGGPSVFLFPPKP | |
| | KDTLMISRTPEVTCVVVDVSHEDPEVKFNWYVDGVEVHNAKTKPRE | |
| | EQYNSTYRVVSVLTVLHQDWLNGKEYKCKVSNKALPAPIEKTISKA | |
| | KGQPREPQVYTLPPSRDELTKNQVSLTCLVKGFYPSDIAVEWESNG | |
| | QPENNYKTTPPVLDSDGSFFLYSKLTVDKSRWQQGNVFSCSVMHEA | |
| | LHNHYTQKSLSLSPGK | |

The 42 LFc V1 is a variant in which an amino acid residue substitution of G132R in the sequence of 42 LFc occurred; the LFc V2 is a variant in which amino acid residue substitutions of G132R and R135G in the sequence of 42 LFc occurred; and the 42 LFc V3 is a variant in which amino acid residue substitutions of G132R and G134E in the sequence of 42 LFc occurred. The 42 LFc V4 is a variant in which an amino acid residue substitution of R135G in the sequence of 42 LFc occurred; the 42 LFc V5 is a variant in which amino acid residue substitutions of G74R and G77A in the sequence of 42 LFc occurred; the 42 LFc V6 is a variant in which amino acid residue substitutions of G74R, G77A, G132R and R135A in the sequence of 42 LFc occurred; the 42 LFc V7 is a variant in which amino acid residue substitutions of G74R, G77A, G132R and R135V in the sequence of 42 LFc occurred; the 42 LFc V8 is a variant in which amino acid residue substitutions of G74R and G77V in the sequence of 42 LFc occurred; the 42 LFc V9 is a variant in which amino acid residue substitutions of G74R, G77V, G132R and R135A in the sequence of 42 LFc occurred; the 42 LFc V10 is a variant in which amino acid residue substitutions of G74R, G77V, G132R and R135V in the sequence of 42 LFc occurred; the 42 LFc V11 is a variant in which amino acid residue substitutions of G74R, G75H, K76G, G132R and R135G in the sequence of 42 LFc occurred; the 42 LFc V12 is a variant in which amino acid residue substitutions of G74R, G132R and R135G in the sequence of 42 LFc occurred; and the 42 LFc V13 is a variant in which amino acid residue substitutions of G74R, G77A, G132R and R135G in the sequence of 42 LFc occurred.

Table 7 below shows the amino acid sequences of variants in which the following substitutions of amino acid residues occurred: a substitution of amino acid residues in the propeptide domain (L22 to S73) as shown in Table 5 above; a substitution of amino acid residues at the processing sites (R74 to R77 and R132 to R135) as shown in Table 6 above; and an additional substitution of an amino acid residue at a fragmentation site (S202 or S223).

TABLE 7
Amino acid sequences of Lefty A fusion protein
variants (combination)

| | | SEQ |
|---|---|---|
| | | ID |
| | Amino acid sequences | NOS: |
| CX196 | MWPLWLCWALWVLPLAGPGAALTGEQLLGSLLRQLQLKEVPTLDR | 105 |
| | ADMEELVIPTHVRAQYVALLQRSHGDRSGGKGFSQSFREVAGRFL | |
| | ASEASTHLLVFGMEQRLPPNSELVQAVLRLFQEPVPKAALHRHGG | |
| | LSPRSAQARVTVEWLRVRDDGSNRTSLIDSRLVSVHESGWKAFDV | |
| | TEAVNFWQQLSRPRQPLLLQVSVQREHLGPLASGAHKLVRFASQG | |
| | APAGLGEPQLELHTLDLRDYGAQGDCDPEAPMTEGTRCCRQEMYI | |
| | DLQGMKWAKNWVLEPPGFLAYECVGTCQQPPEALAFNWPFLGPRQ | |
| | CIASETASLPMIVSIKEGGRTRPQVVSLPNMRVQKCSCASDGALV | |
| | PRRLQPSGGGGSGGGGSGGGGSEPKSCDKTHTCPPCPAPELLGGP | |
| | SVFLFPPKPKDTLMISRTPEVTCVVVDVSHEDPEVKFNWYVDGVE | |
| | VHNAKTKPREEQYNSTYRVVSVLTVLHQDWLNGKEYKCKVSNKAL | |
| | PAPIEKTISKAKGQPREPQVYTLPPSRDELTKNQVSLTCLVKGFY | |
| | PSDIAVEWESNGQPENNYKTTPPVLDSDGSFFLYSKLTVDKSRWQ | |
| | QGNVFSCSVMHEALHNHYTQKSLSLSPGK | |
| CX197 | MWPLWLCWALWVLPLAGPGAALTGEQLLGSLLRQLQLKEVPTLDR | 106 |
| | ADMEELVIPTHVRAQYVALLQRSHGDRSGGKGFSQSFREVAGRFL | |
| | ASEASTHLLVFGMEQRLPPNSELVQAVLRLFQEPVPKAALHRHGA | |
| | LSPRSAQARVTVEWLRVRDDGSNRTSLIDSRLVSVHESGWKAFDV | |
| | TEAVNFWQQLSRPRQPLLLQVSVQREHLGPLASGAHKLVRFASQG | |
| | APAGLGEPQLELHTLDLRDYGAQGDCDPEAPMTEGTRCCRQEMYI | |
| | DLQGMKWAKNWVLEPPGFLAYECVGTCQQPPEALAFNWPFLGPRQ | |
| | CIASETASLPMIVSIKEGGRTRPQVVSLPNMRVQKCSCASDGALV | |
| | PRRLQPSGGGGSGGGGSGGGGSEPKSCDKTHTCPPCPAPELLGGP | |
| | SVFLFPPKPKDTLMISRTPEVTCVVVDVSHEDPEVKFNWYVDGVE | |
| | VHNAKTKPREEQYNSTYRVVSVLTVLHQDWLNGKEYKCKVSNKAL | |
| | PAPIEKTISKAKGQPREPQVYTLPPSRDELTKNQVSLTCLVKGFY | |
| | PSDIAVEWESNGQPENNYKTTPPVLDSDGSFFLYSKLTVDKSRWQ | |
| | QGNVFSCSVMHEALHNHYTQKSLSLSPGK | |
| CX201 | MWPLWLCWALWVLPLAGPGAALTEEQLLGSLLRQLQLSEVPVLDR | 107 |
| | ADMEKLVIPAHVRAQYVALLQRSHGDRSGGKGFSQSFREVAGRFL | |
| | ASEASTHLLVFGMEQRLPPNSELVQAVLRLFQEPVPKAALHRHGG | |
| | LSPRSAQARVTVEWLRVRDDGSNRTSLIDSRLVSVHESGWKAFDV | |
| | TEAVNFWQQLSRPRQPLLLQVSVQREHLGPLASGAHKLVRFASQG | |
| | APAGLGEPQLELHTLDLRDYGAQGDCDPEAPMTEGTRCCRQEMYI | |
| | DLQGMKWAKNWVLEPPGFLAYECVGTCQQPPEALAFNWPFLGPRQ | |
| | CIASETASLPMIVSIKEGGRTRPQVVSLPNMRVQKCSCASDGALV | |
| | PRRLQPSGGGGSGGGGSGGGGSEPKSCDKTHTCPPCPAPELLGGP | |
| | SVFLFPPKPKDTLMISRTPEVTCVVVDVSHEDPEVKFNWYVDGVE | |
| | VHNAKTKPREEQYNSTYRVVSVLTVLHQDWLNGKEYKCKVSNKAL | |
| | PAPIEKTISKAKGQPREPQVYTLPPSRDELTKNQVSLTCLVKGFY | |
| | PSDIAVEWESNGQPENNYKTTPPVLDSDGSFFLYSKLTVDKSRWQ | |
| | QGNVFSCSVMHEALHNHYTQKSLSLSPGK | |
| CX203 | MWPLWLCWALWVLPLAGPGAALTEEQLLGSLLRQLQLSEVPVLDR | 108 |
| | ADMEKLVIPAHVRAQYVALLQRSHGDRSGGKGFSQSFREVAGRFL | |
| | ASEASTHLLVFGMEQRLPPNSELVQAVLRLFQEPVPKAALHRHER | |
| | LSPRSAQARVTVEWLRVRDDGSNRTSLIDSRLVSVHESGWKAFDV | |
| | TEAVNFWQQLSRPRQPLLLQVSVQREHLGPLASGAHKLVRFASQG | |
| | APAGLGEPQLELHTLDLRDYGAQGDCDPEAPMTEGTRCCRQEMYI | |
| | DLQGMKWAKNWVLEPPGFLAYECVGTCQQPPEALAFNWPFLGPRQ | |
| | CIASETASLPMIVSIKEGGRTRPQVVSLPNMRVQKCSCASDGALV | |
| | PRRLQPSGGGGSGGGGSGGGGSEPKSCDKTHTCPPCPAPELLGGP | |
| | SVFLFPPKPKDTLMISRTPEVTCVVVDVSHEDPEVKFNWYVDGVE | |
| | VHNAKTKPREEQYNSTYRVVSVLTVLHQDWLNGKEYKCKVSNKAL | |
| | PAPIEKTISKAKGQPREPQVYTLPPSRDELTKNQVSLTCLVKGFY | |
| | PSDIAVEWESNGQPENNYKTTPPVLDSDGSFFLYSKLTVDKSRWQ | |
| | QGNVFSCSVMHEALHNHYTQKSLSLSPGK | |
| CX206 | MWPLWLCWALWVLPLAGPGAALTEEQLLGSLLRQLQLSEVPVLDR | 109 |
| | ADMEKLVIPAHVRAQYVALLQRSHGDRSGGKGFSQSFREVAGRFL | |
| | ASEASTHLLVFGMEQRLPPNSELVQAVLRLFQEPVPKAALHRHGG | |
| | LSPRSAQARVTVEWLRVRDDGSNRTSLIDSRLVSVHESGWKAFDV | |
| | TEAVNFWQQLSRPRQPLLLQVTVQREHLGPLASGAHKLVRFASQG | |
| | APAGLGEPQLELHTLDLRDYGAQGDCDPEAPMTEGTRCCRQEMYI | |
| | DLQGMKWAKNWVLEPPGFLAYECVGTCQQPPEALAFNWPFLGPRQ | |
| | CIASETASLPMIVSIKEGGRTRPQVVSLPNMRVQKCSCASDGALV | |
| | PRRLQPSGGGGSGGGGSGGGGSEPKSCDKTHTCPPCPAPELLGGP | |
| | SVFLFPPKPKDTLMISRTPEVTCVVVDVSHEDPEVKFNWYVDGVE | |
| | VHNAKTKPREEQYNSTYRVVSVLTVLHQDWLNGKEYKCKVSNKAL | |
| | PAPIEKTISKAKGQPREPQVYTLPPSRDELTKNQVSLTCLVKGFY | |
| | PSDIAVEWESNGQPENNYKTTPPVLDSDGSFFLYSKLTVDKSRWQ | |
| | QGNVFSCSVMHEALHNHYTQKSLSLSPGK | |
| CX207 | MWPLWLCWALWVLPLAGPGAALTEEQLLGSLLRQLQLSEVPVLDR | 110 |
| | ADMEKLVIPAHVRAQYVALLQRSHGDRSGGKGFSQSFREVAGRFL | |
| | ASEASTHLLVFGMEQRLPPNSELVQAVLRLFQEPVPKAALHRHGG | |
| | LSPRSAQARVTVEWLRVRDDGSNRTSLIDSRLVSVHESGWKAFDV | |
| | TEAVNFWQQLSRPRQPLLLQVSVQREHLGPLASGAHKLVRFAGQG | |
| | APAGLGEPQLELHTLDLRDYGAQGDCDPEAPMTEGTRCCRQEMYI | |
| | DLQGMKWAKNWVLEPPGFLAYECVGTCQQPPEALAFNWPFLGPRQ | |
| | CIASETASLPMIVSIKEGGRTRPQVVSLPNMRVQKCSCASDGALV | |
| | PRRLQPSGGGGSGGGGSGGGGSEPKSCDKTHTCPPCPAPELLGGP | |
| | SVFLFPPKPKDTLMISRTPEVTCVVVDVSHEDPEVKFNWYVDGVE | |
| | VHNAKTKPREEQYNSTYRVVSVLTVLHQDWLNGKEYKCKVSNKAL | |
| | PAPIEKTISKAKGQPREPQVYTLPPSRDELTKNQVSLTCLVKGFY | |
| | PSDIAVEWESNGQPENNYKTTPPVLDSDGSFFLYSKLTVDKSRWQ | |
| | QGNVFSCSVMHEALHNHYTQKSLSLSPGK | |
| CX208 | MWPLWLCWALWVLPLAGPGAALTEEQLLGSLLRQLQLSEVPVLDR | 111 |
| | ADMEKLVIPAHVRAQYVALLQRSHGDRSGGKGFSQSFREVAGRFL | |
| | ASEASTHLLVFGMEQRLPPNSELVQAVLRLFQEPVPKAALHRHGG | |
| | LSPRSAQARVTVEWLRVRDDGSNRTSLIDSRLVSVHESGWKAFDV | |
| | TEAVNFWQQLSRPRQPLLLQVTVQREHLGPLASGAHKLVRFAGQG | |
| | APAGLGEPQLELHTLDLRDYGAQGDCDPEAPMTEGTRCCRQEMYI | |
| | DLQGMKWAKNWVLEPPGFLAYECVGTCQQPPEALAFNWPFLGPRQ | |
| | CIASETASLPMIVSIKEGGRTRPQVVSLPNMRVQKCSCASDGALV | |
| | PRRLQPSGGGGSGGGGSGGGGSEPKSCDKTHTCPPCPAPELLGGP | |
| | SVFLFPPKPKDTLMISRTPEVTCVVVDVSHEDPEVKFNWYVDGVE | |
| | VHNAKTKPREEQYNSTYRVVSVLTVLHQDWLNGKEYKCKVSNKAL | |
| | PAPIEKTISKAKGQPREPQVYTLPPSRDELTKNQVSLTCLVKGFY | |
| | PSDIAVEWESNGQPENNYKTTPPVLDSDGSFFLYSKLTVDKSRWQ | |
| | QGNVFSCSVMHEALHNHYTQKSLSLSPGK | |

The CX196 is a variant in which amino acid residue substitutions of E24G, S38K, V42T, K50E, A55T, V63A, R66Q, G132R and R135G in the sequence of 42 LFc occurred, and the CX197 is a variant in which amino acid residue substitutions of E24G, S38K, V42T, K50E, A55T, V63A, R66Q, G132R and R135A in the sequence of 42 LFc occurred.

The CX201 is a variant in which amino acid residue substitutions of V63A, R66Q, G132R and R135A in the sequence of 42 LFc occurred, and the CX203 is a variant in which amino acid residue substitutions of V63A, R66Q, G132R and G134E in the sequence of 42 LFc occurred.

The CX206 is a variant in which amino acid residue substitutions of V63A, R66Q, G132R, R135G and S202T in the sequence of 42 LFc occurred, the CX207 is a variant in which amino acid residue substitutions of V63A, R66Q, G132R, R135G and S223G in the sequence of 42 LFc occurred, and the CX208 is a variant in which amino acid residue substitutions of V63A, R66Q, G132R, R135G, S202T and S223G in the sequence of 42 LFc occurred.

Table 8 below shows a variant in which a substitution of amino acid residues at the thrombin cleavage site (L311, P313, R314, L359, P361 or R362) in the sequence of CX201 shown in Table 7 above occurred.

TABLE 8
Amino acid sequence of human Lefty A fusion
protein variant (thrombin cleavage site)

| | | SEQ |
|---|---|---|
| | | ID |
| | Amino acid sequences | NOS: |
| CX201 | MDMRVPAQLLGLLLLWFPGSRCLTEEQLLGSLLRQLQLSEVPV | 112 |
| (L311D) | LDRADMEKLVIPAHVRAQYVALLQRSHGDRSGGKGFSQSFREV | |
| | AGRFLASEASTHLLVFGMEQRLPPNSELVQAVLRLFQEPVPKA | |
| | ALHRHGGLSPRSAQARVTVEWLRVRDDGSNRTSLIDSRLVSVH | |
| | ESGWKAFDVTEAVNFWQQLSRPRQPLLLQVSVQREHLGPLASG | |
| | AHKLVRFASQGAPAGLGEPQLELHTLDLRDYGAQGDCDPEAPM | |
| | TEGTRCCRQEMYIDLQGMKWAKNWVLEPPGFLAYECVGTCQQP | |
| | PEALAFNWPFDGPRQCIASETASLPMIVSIKEGGRTRPQVVSL | |
| | PNMRVQKCSCASDGALVPRRLQPSGGGGSGGGGSGGGGSEPKS | |
| | CDKTHTCPPCPAPELLGGPSVFLFPPKPKDTLMISRTPEVTCV | |
| | VVDVSHEDPEVKFNWYVDGVEVHNAKTKPREEQYNSTYRVVSV | |
| | LTVLHQDWLNGKEYKCKVSNKALPAPIEKTISKAKGQPREPQV | |
| | YTLPPSRDELTKNQVSLTCLVKGFYPSDIAVEWESNGQPENNY | |
| | KTTPPVLDSDGSFFLYSKLTVDKSRWQQGNVFSCSVMHEALHN | |
| | HYTQKSLSLSPGK | |
| CX201 | MDMRVPAQLLGLLLLWFPGSRCLTEEQLLGSLLRQLQLSEVPV | 113 |
| (L311E) | LDRADMEKLVIPAHVRAQYVALLQRSHGDRSGGKGFSQSFREV | |
| | AGRFLASEASTHLLVFGMEQRLPPNSELVQAVLRLFQEPVPKA | |
| | ALHRHGGLSPRSAQARVTVEWLRVRDDGSNRTSLIDSRLVSVH | |
| | ESGWKAFDVTEAVNFWQQLSRPRQPLLLQVSVQREHLGPLASG | |
| | AHKLVRFASQGAPAGLGEPQLELHTLDLRDYGAQGDCDPEAPM | |
| | TEGTRCCRQEMYIDLQGMKWAKNWVLEPPGFLAYECVGTCQQP | |
| | PEALAFNWPFEGPRQCIASETASLPMIVSIKEGGRTRPQVVSL | |
| | PNMRVQKCSCASDGALVPRRLQPSGGGGSGGGGSGGGGSEPKS | |
| | CDKTHTCPPCPAPELLGGPSVFLFPPKPKDTLMISRTPEVTCV | |
| | VVDVSHEDPEVKFNWYVDGVEVHNAKTKPREEQYNSTYRVVSV | |
| | LTVLHQDWLNGKEYKCKVSNKALPAPIEKTISKAKGQPREPQV | |
| | YTLPPSRDELTKNQVSLTCLVKGFYPSDIAVEWESNGQPENNY | |
| | KTTPPVLDSDGSFFLYSKLTVDKSRWQQGNVFSCSVMHEALHN | |
| | HYTQKSLSLSPGK | |
| CX201 | MDMRVPAQLLGLLLLWFPGSRCLTEEQLLGSLLRQLQLSEVPV | 114 |
| (P313D) | LDRADMEKLVIPAHVRAQYVALLQRSHGDRSGGKGFSQSFREV | |
| | AGRFLASEASTHLLVFGMEQRLPPNSELVQAVLRLFQEPVPKA | |
| | ALHRHGGLSPRSAQARVTVEWLRVRDDGSNRTSLIDSRLVSVH | |
| | ESGWKAFDVTEAVNFWQQLSRPRQPLLLQVSVQREHLGPLASG | |
| | AHKLVRFASQGAPAGLGEPQLELHTLDLRDYGAQGDCDPEAPM | |
| | TEGTRCCRQEMYIDLQGMKWAKNWVLEPPGFLAYECVGTCQQP | |
| | PEALAFNWPFLGDRQCIASETASLPMIVSIKEGGRTRPQVVSL | |
| | PNMRVQKCSCASDGALVPRRLQPSGGGGSGGGGSGGGGSEPKS | |
| | CDKTHTCPPCPAPELLGGPSVFLFPPKPKDTLMISRTPEVTCV | |
| | VVDVSHEDPEVKFNWYVDGVEVHNAKTKPREEQYNSTYRVVSV | |
| | LTVLHQDWLNGKEYKCKVSNKALPAPIEKTISKAKGQPREPQV | |
| | YTLPPSRDELTKNQVSLTCLVKGFYPSDIAVEWESNGQPENNY | |
| | KTTPPVLDSDGSFFLYSKLTVDKSRWQQGNVFSCSVMHEALHN | |
| | HYTQKSLSLSPGK | |
| CX201 | MDMRVPAQLLGLLLLWFPGSRCLTEEQLLGSLLRQLQLSEVPV | 115 |
| (P313E) | LDRADMEKLVIPAHVRAQYVALLQRSHGDRSGGKGFSQSFREV | |
| | AGRFLASEASTHLLVFGMEQRLPPNSELVQAVLRLFQEPVPKA | |
| | ALHRHGGLSPRSAQARVTVEWLRVRDDGSNRTSLIDSRLVSVH | |
| | ESGWKAFDVTEAVNFWQQLSRPRQPLLLQVSVQREHLGPLASG | |
| | AHKLVRFASQGAPAGLGEPQLELHTLDLRDYGAQGDCDPEAPM | |
| | TEGTRCCRQEMYIDLQGMKWAKNWVLEPPGFLAYECVGTCQQP | |
| | PEALAFNWPFLGERQCIASETASLPMIVSIKEGGRTRPQVVSL | |
| | PNMRVQKCSCASDGALVPRRLQPSGGGGSGGGGSGGGGSEPKS | |
| | CDKTHTCPPCPAPELLGGPSVFLFPPKPKDTLMISRTPEVTCV | |
| | VVDVSHEDPEVKFNWYVDGVEVHNAKTKPREEQYNSTYRVVSV | |
| | LTVLHQDWLNGKEYKCKVSNKALPAPIEKTISKAKGQPREPQV | |
| | YTLPPSRDELTKNQVSLTCLVKGFYPSDIAVEWESNGQPENNY | |
| | KTTPPVLDSDGSFFLYSKLTVDKSRWQQGNVFSCSVMHEALHN | |
| | HYTQKSLSLSPGK | |
| CX201 | MDMRVPAQLLGLLLLWFPGSRCLTEEQLLGSLLRQLQLSEVPV | 116 |
| (P313S) | LDRADMEKLVIPAHVRAQYVALLQRSHGDRSGGKGFSQSFREV | |
| | AGRFLASEASTHLLVFGMEQRLPPNSELVQAVLRLFQEPVPKA | |
| | ALHRHGGLSPRSAQARVTVEWLRVRDDGSNRTSLIDSRLVSVH | |
| | ESGWKAFDVTEAVNFWQQLSRPRQPLLLQVSVQREHLGPLASG | |
| | AHKLVRFASQGAPAGLGEPQLELHTLDLRDYGAQGDCDPEAPM | |
| | TEGTRCCRQEMYIDLQGMKWAKNWVLEPPGFLAYECVGTCQQP | |
| | PEALAFNWPFLGSRQCIASETASLPMIVSIKEGGRTRPQVVSL | |
| | PNMRVQKCSCASDGALVPRRLQPSGGGGSGGGGSGGGGSEPKS | |
| | CDKTHTCPPCPAPELLGGPSVFLFPPKPKDTLMISRTPEVTCV | |
| | VVDVSHEDPEVKFNWYVDGVEVHNAKTKPREEQYNSTYRVVSV | |
| | LTVLHQDWLNGKEYKCKVSNKALPAPIEKTISKAKGQPREPQV | |
| | YTLPPSRDELTKNQVSLTCLVKGFYPSDIAVEWESNGQPENNY | |
| | KTTPPVLDSDGSFFLYSKLTVDKSRWQQGNVFSCSVMHEALHN | |
| | HYTQKSLSLSPGK | |
| CX201 | MDMRVPAQLLGLLLLWFPGSRCLTEEQLLGSLLRQLQLSEVPV | 117 |
| (R314K) | LDRADMEKLVIPAHVRAQYVALLQRSHGDRSGGKGFSQSFREV | |
| | AGRFLASEASTHLLVFGMEQRLPPNSELVQAVLRLFQEPVPKA | |
| | ALHRHGGLSPRSAQARVTVEWLRVRDDGSNRTSLIDSRLVSVH | |
| | ESGWKAFDVTEAVNFWQQLSRPRQPLLLQVSVQREHLGPLASG | |
| | AHKLVRFASQGAPAGLGEPQLELHTLDLRDYGAQGDCDPEAPM | |
| | TEGTRCCRQEMYIDLQGMKWAKNWVLEPPGFLAYECVGTCQQP | |
| | PEALAFNWPFLGPKQCIASETASLPMIVSIKEGGRTRPQVVSL | |
| | PNMRVQKCSCASDGALVPRRLQPSGGGGSGGGGSGGGGSEPKS | |
| | CDKTHTCPPCPAPELLGGPSVFLFPPKPKDTLMISRTPEVTCV | |
| | VVDVSHEDPEVKFNWYVDGVEVHNAKTKPREEQYNSTYRVVSV | |
| | LTVLHQDWLNGKEYKCKVSNKALPAPIEKTISKAKGQPREPQV | |
| | YTLPPSRDELTKNQVSLTCLVKGFYPSDIAVEWESNGQPENNY | |
| | KTTPPVLDSDGSFFLYSKLTVDKSRWQQGNVFSCSVMHEALHN | |
| | HYTQKSLSLSPGK | |
| CX201 | MDMRVPAQLLGLLLLWFPGSRCLTEEQLLGSLLRQLQLSEVPV | 118 |
| (R314Q) | LDRADMEKLVIPAHVRAQYVALLQRSHGDRSGGKGFSQSFREV | |
| | AGRFLASEASTHLLVFGMEQRLPPNSELVQAVLRLFQEPVPKA | |
| | ALHRHGGLSPRSAQARVTVEWLRVRDDGSNRTSLIDSRLVSVH | |
| | ESGWKAFDVTEAVNFWQQLSRPRQPLLLQVSVQREHLGPLASG | |
| | AHKLVRFASQGAPAGLGEPQLELHTLDLRDYGAQGDCDPEAPM | |
| | TEGTRCCRQEMYIDLQGMKWAKNWVLEPPGFLAYECVGTCQQP | |
| | PEALAFNWPFLGPQQCIASETASLPMIVSIKEGGRTRPQVVSL | |
| | PNMRVQKCSCASDGALVPRRLQPSGGGGSGGGGSGGGGSEPKS | |
| | CDKTHTCPPCPAPELLGGPSVFLFPPKPKDTLMISRTPEVTCV | |
| | VVDVSHEDPEVKFNWYVDGVEVHNAKTKPREEQYNSTYRVVSV | |
| | LTVLHQDWLNGKEYKCKVSNKALPAPIEKTISKAKGQPREPQV | |
| | YTLPPSRDELTKNQVSLTCLVKGFYPSDIAVEWESNGQPENNY | |
| | KTTPPVLDSDGSFFLYSKLTVDKSRWQQGNVFSCSVMHEALHN | |
| | HYTQKSLSLSPGK | |
| CX201 | MDMRVPAQLLGLLLLWFPGSRCLTEEQLLGSLLRQLQLSEVPV | 119 |
| (L359D) | LDRADMEKLVIPAHVRAQYVALLQRSHGDRSGGKGFSQSFREV | |
| | AGRFLASEASTHLLVFGMEQRLPPNSELVQAVLRLFQEPVPKA | |
| | ALHRHGGLSPRSAQARVTVEWLRVRDDGSNRTSLIDSRLVSVH | |
| | ESGWKAFDVTEAVNFWQQLSRPRQPLLLQVSVQREHLGPLASG | |
| | AHKLVRFASQGAPAGLGEPQLELHTLDLRDYGAQGDCDPEAPM | |
| | TEGTRCCRQEMYIDLQGMKWAKNWVLEPPGFLAYECVGTCQQP | |
| | PEALAFNWPFLGPRQCIASETASLPMIVSIKEGGRTRPQVVSL | |
| | PNMRVQKCSCASDGADVPRRLQPSGGGGSGGGGSGGGGSEPKS | |
| | CDKTHTCPPCPAPELLGGPSVFLFPPKPKDTLMISRTPEVTCV | |
| | VVDVSHEDPEVKFNWYVDGVEVHNAKTKPREEQYNSTYRVVSV | |
| | LTVLHQDWLNGKEYKCKVSNKALPAPIEKTISKAKGQPREPQV | |
| | YTLPPSRDELTKNQVSLTCLVKGFYPSDIAVEWESNGQPENNY | |
| | KTTPPVLDSDGSFFLYSKLTVDKSRWQQGNVFSCSVMHEALHN | |
| | HYTQKSLSLSPGK | |
| CX201 | MDMRVPAQLLGLLLLWFPGSRCLTEEQLLGSLLRQLQLSEVPV | 120 |
| (L359E) | LDRADMEKLVIPAHVRAQYVALLQRSHGDRSGGKGFSQSFREV | |
| | AGRFLASEASTHLLVFGMEQRLPPNSELVQAVLRLFQEPVPKA | |
| | ALHRHGGLSPRSAQARVTVEWLRVRDDGSNRTSLIDSRLVSVH | |
| | ESGWKAFDVTEAVNFWQQLSRPRQPLLLQVSVQREHLGPLASG | |
| | AHKLVRFASQGAPAGLGEPQLELHTLDLRDYGAQGDCDPEAPM | |
| | TEGTRCCRQEMYIDLQGMKWAKNWVLEPPGFLAYECVGTCQQP | |
| | PEALAFNWPFLGPRQCIASETASLPMIVSIKEGGRTRPQVVSL | |
| | PNMRVQKCSCASDGAEVPRRLQPSGGGGSGGGGSGGGGSEPKS | |
| | CDKTHTCPPCPAPELLGGPSVFLFPPKPKDTLMISRTPEVTCV | |
| | VVDVSHEDPEVKFNWYVDGVEVHNAKTKPREEQYNSTYRVVSV | |
| | LTVLHQDWLNGKEYKCKVSNKALPAPIEKTISKAKGQPREPQV | |
| | YTLPPSRDELTKNQVSLTCLVKGFYPSDIAVEWESNGQPENNY | |
| | KTTPPVLDSDGSFFLYSKLTVDKSRWQQGNVFSCSVMHEALHN | |
| | HYTQKSLSLSPGK | |
| CX201 | MDMRVPAQLLGLLLLWFPGSRCLTEEQLLGSLLRQLQLSEVPV | 121 |
| (P361D) | LDRADMEKLVIPAHVRAQYVALLQRSHGDRSGGKGFSQSFREV | |
| | AGRFLASEASTHLLVFGMEQRLPPNSELVQAVLRLFQEPVPKA | |
| | ALHRHGGLSPRSAQARVTVEWLRVRDDGSNRTSLIDSRLVSVH | |
| | ESGWKAFDVTEAVNFWQQLSRPRQPLLLQVSVQREHLGPLASG | |
| | AHKLVRFASQGAPAGLGEPQLELHTLDLRDYGAQGDCDPEAPM | |
| | TEGTRCCRQEMYIDLQGMKWAKNWVLEPPGFLAYECVGTCQQP | |
| | PEALAFNWPFLGPRQCIASETASLPMIVSIKEGGRTRPQVVSL | |
| | PNMRVQKCSCASDGALVDRRLQPSGGGGSGGGGSGGGGSEPKS | |
| | CDKTHTCPPCPAPELLGGPSVFLFPPKPKDTLMISRTPEVTCV | |
| | VVDVSHEDPEVKFNWYVDGVEVHNAKTKPREEQYNSTYRVVSV | |
| | LTVLHQDWLNGKEYKCKVSNKALPAPIEKTISKAKGQPREPQV | |
| | YTLPPSRDELTKNQVSLTCLVKGFYPSDIAVEWESNGQPENNY | |
| | KTTPPVLDSDGSFFLYSKLTVDKSRWQQGNVFSCSVMHEALHN | |
| | HYTQKSLSLSPGK | |
| CX201 | MDMRVPAQLLGLLLLWFPGSRCLTEEQLLGSLLRQLQLSEVPV | 122 |
| (P361E) | LDRADMEKLVIPAHVRAQYVALLQRSHGDRSGGKGFSQSFREV | |
| | AGRFLASEASTHLLVFGMEQRLPPNSELVQAVLRLFQEPVPKA | |
| | ALHRHGGLSPRSAQARVTVEWLRVRDDGSNRTSLIDSRLVSVH | |
| | ESGWKAFDVTEAVNFWQQLSRPRQPLLLQVSVQREHLGPLASG | |
| | AHKLVRFASQGAPAGLGEPQLELHTLDLRDYGAQGDCDPEAPM | |
| | TEGTRCCRQEMYIDLQGMKWAKNWVLEPPGFLAYECVGTCQQP | |
| | PEALAFNWPFLGPRQCIASETASLPMIVSIKEGGRTRPQVVSL | |
| | PNMRVQKCSCASDGALVERRLQPSGGGGSGGGGSGGGGSEPKS | |
| | CDKTHTCPPCPAPELLGGPSVFLFPPKPKDTLMISRTPEVTCV | |
| | VVDVSHEDPEVKFNWYVDGVEVHNAKTKPREEQYNSTYRVVSV | |
| | LTVLHQDWLNGKEYKCKVSNKALPAPIEKTISKAKGQPREPQV | |
| | YTLPPSRDELTKNQVSLTCLVKGFYPSDIAVEWESNGQPENNY | |
| | KTTPPVLDSDGSFFLYSKLTVDKSRWQQGNVFSCSVMHEALHN | |
| | HYTQKSLSLSPGK | |
| CX201 | MDMRVPAQLLGLLLLWFPGSRCLTEEQLLGSLLRQLQLSEVPV | 123 |
| (P361S) | LDRADMEKLVIPAHVRAQYVALLQRSHGDRSGGKGFSQSFREV | |
| | AGRFLASEASTHLLVFGMEQRLPPNSELVQAVLRLFQEPVPKA | |
| | ALHRHGGLSPRSAQARVTVEWLRVRDDGSNRTSLIDSRLVSVH | |
| | ESGWKAFDVTEAVNFWQQLSRPRQPLLLQVSVQREHLGPLASG | |
| | AHKLVRFASQGAPAGLGEPQLELHTLDLRDYGAQGDCDPEAPM | |
| | TEGTRCCRQEMYIDLQGMKWAKNWVLEPPGFLAYECVGTCQQP | |
| | PEALAFNWPFLGPRQCIASETASLPMIVSIKEGGRTRPQVVSL | |
| | PNMRVQKCSCASDGALVSRRLQPSGGGGSGGGGSGGGGSEPKS | |
| | CDKTHTCPPCPAPELLGGPSVFLFPPKPKDTLMISRTPEVTCV | |
| | VVDVSHEDPEVKFNWYVDGVEVHNAKTKPREEQYNSTYRVVSV | |
| | LTVLHQDWLNGKEYKCKVSNKALPAPIEKTISKAKGQPREPQV | |
| | YTLPPSRDELTKNQVSLTCLVKGFYPSDIAVEWESNGQPENNY | |
| | KTTPPVLDSDGSFFLYSKLTVDKSRWQQGNVFSCSVMHEALHN | |
| | HYTQKSLSLSPGK | |
| CX201 | MDMRVPAQLLGLLLLWFPGSRCLTEEQLLGSLLRQLQLSEVPV | 124 |
| (R362K) | LDRADMEKLVIPAHVRAQYVALLQRSHGDRSGGKGFSQSFREV | |
| | AGRFLASEASTHLLVFGMEQRLPPNSELVQAVLRLFQEPVPKA | |
| | ALHRHGGLSPRSAQARVTVEWLRVRDDGSNRTSLIDSRLVSVH | |
| | ESGWKAFDVTEAVNFWQQLSRPRQPLLLQVSVQREHLGPLASG | |
| | AHKLVRFASQGAPAGLGEPQLELHTLDLRDYGAQGDCDPEAPM | |
| | TEGTRCCRQEMYIDLQGMKWAKNWVLEPPGFLAYECVGTCQQP | |
| | PEALAFNWPFLGPRQCIASETASLPMIVSIKEGGRTRPQVVSL | |
| | PNMRVQKCSCASDGALVPKRLQPSGGGGSGGGGSGGGGSEPKS | |
| | CDKTHTCPPCPAPELLGGPSVFLFPPKPKDTLMISRTPEVTCV | |
| | VVDVSHEDPEVKFNWYVDGVEVHNAKTKPREEQYNSTYRVVSV | |
| | LTVLHQDWLNGKEYKCKVSNKALPAPIEKTISKAKGQPREPQV | |
| | YTLPPSRDELTKNQVSLTCLVKGFYPSDIAVEWESNGQPENNY | |
| | KTTPPVLDSDGSFFLYSKLTVDKSRWQQGNVFSCSVMHEALHN | |
| | HYTQKSLSLSPGK | |
| CX201 | MDMRVPAQLLGLLLLWFPGSRCLTEEQLLGSLLRQLQLSEVPV | 125 |
| (R362Q) | LDRADMEKLVIPAHVRAQYVALLQRSHGDRSGGKGFSQSFREV | |
| | AGRFLASEASTHLLVFGMEQRLPPNSELVQAVLRLFQEPVPKA | |
| | ALHRHGGLSPRSAQARVTVEWLRVRDDGSNRTSLIDSRLVSVH | |
| | ESGWKAFDVTEAVNFWQQLSRPRQPLLLQVSVQREHLGPLASG | |
| | AHKLVRFASQGAPAGLGEPQLELHTLDLRDYGAQGDCDPEAPM | |
| | TEGTRCCRQEMYIDLQGMKWAKNWVLEPPGFLAYECVGTCQQP | |
| | PEALAFNWPFLGPRQCIASETASLPMIVSIKEGGRTRPQVVSL | |
| | PNMRVQKCSCASDGALVPQRLQPSGGGGSGGGGSGGGGSEPKS | |
| | CDKTHTCPPCPAPELLGGPSVFLFPPKPKDTLMISRTPEVTCV | |
| | VVDVSHEDPEVKFNWYVDGVEVHNAKTKPREEQYNSTYRVVSV | |
| | LTVLHQDWLNGKEYKCKVSNKALPAPIEKTISKAKGQPREPQV | |
| | YTLPPSRDELTKNQVSLTCLVKGFYPSDIAVEWESNGQPENNY | |
| | KTTPPVLDSDGSFFLYSKLTVDKSRWQQGNVFSCSVMHEALHN | |
| | HYTQKSLSLSPGK | |
| CX201 | MDMRVPAQLLGLLLLWFPGSRCLTEEQLLGSLLRQLQLSEVPV | 126 |
| (L311D/ | LDRADMEKLVIPAHVRAQYVALLQRSHGDRSGGKGFSQSFREV | |
| R362Q) | AGRFLASEASTHLLVFGMEQRLPPNSELVQAVLRLFQEPVPKA | |
| | ALHRHGGLSPRSAQARVTVEWLRVRDDGSNRTSLIDSRLVSVH | |
| | ESGWKAFDVTEAVNFWQQLSRPRQPLLLQVSVQREHLGPLASG | |
| | AHKLVRFASQGAPAGLGEPQLELHTLDLRDYGAQGDCDPEAPM | |
| | TEGTRCCRQEMYIDLQGMKWAKNWVLEPPGFLAYECVGTCQQP | |
| | PEALAFNWPFDGPRQCIASETASLPMIVSIKEGGRTRPQVVSL | |
| | PNMRVQKCSCASDGALVPQRLQPSGGGGSGGGGSGGGGSEPKS | |
| | CDKTHTCPPCPAPELLGGPSVFLFPPKPKDTLMISRTPEVTCV | |
| | VVDVSHEDPEVKFNWYVDGVEVHNAKTKPREEQYNSTYRVVSV | |
| | LTVLHQDWLNGKEYKCKVSNKALPAPIEKTISKAKGQPREPQV | |
| | YTLPPSRDELTKNQVSLTCLVKGFYPSDIAVEWESNGQPENNY | |
| | KTTPPVLDSDGSFFLYSKLTVDKSRWQQGNVFSCSVMHEALHN | |
| | HYTQKSLSLSPGK | |
| CX201 | MDMRVPAQLLGLLLLWFPGSRCLTEEQLLGSLLRQLQLSEVPV | 127 |
| (L311E/ | LDRADMEKLVIPAHVRAQYVALLQRSHGDRSGGKGFSQSFREV | |
| R362Q) | AGRFLASEASTHLLVFGMEQRLPPNSELVQAVLRLFQEPVPKA | |
| | ALHRHGGLSPRSAQARVTVEWLRVRDDGSNRTSLIDSRLVSVH | |
| | ESGWKAFDVTEAVNFWQQLSRPRQPLLLQVSVQREHLGPLASG | |
| | AHKLVRFASQGAPAGLGEPQLELHTLDLRDYGAQGDCDPEAPM | |
| | TEGTRCCRQEMYIDLQGMKWAKNWVLEPPGFLAYECVGTCQQP | |
| | PEALAFNWPFEGPRQCIASETASLPMIVSIKEGGRTRPQVVSL | |
| | PNMRVQKCSCASDGALVPQRLQPSGGGGSGGGGSGGGGSEPKS | |
| | CDKTHTCPPCPAPELLGGPSVFLFPPKPKDTLMISRTPEVTCV | |
| | VVDVSHEDPEVKFNWYVDGVEVHNAKTKPREEQYNSTYRVVSV | |
| | LTVLHQDWLNGKEYKCKVSNKALPAPIEKTISKAKGQPREPQV | |
| | YTLPPSRDELTKNQVSLTCLVKGFYPSDIAVEWESNGQPENNY | |
| | KTTPPVLDSDGSFFLYSKLTVDKSRWQQGNVFSCSVMHEALHN | |
| | HYTQKSLSLSPGK | |
| CX201 | MDMRVPAQLLGLLLLWFPGSRCLTEEQLLGSLLRQLQLSEVPV | 128 |
| (L311D/ | LDRADMEKLVIPAHVRAQYVALLQRSHGDRSGGKGFSQSFREV | |
| P361D/ | AGRFLASEASTHLLVFGMEQRLPPNSELVQAVLRLFQEPVPKA | |
| R362Q) | ALHRHGGLSPRSAQARVTVEWLRVRDDGSNRTSLIDSRLVSVH | |
| | ESGWKAFDVTEAVNFWQQLSRPRQPLLLQVSVQREHLGPLASG | |
| | AHKLVRFASQGAPAGLGEPQLELHTLDLRDYGAQGDCDPEAPM | |
| | TEGTRCCRQEMYIDLQGMKWAKNWVLEPPGFLAYECVGTCQQP | |
| | PEALAFNWPFDGPRQCIASETASLPMIVSIKEGGRTRPQVVSL | |
| | PNMRVQKCSCASDGALVDQRLQPSGGGGSGGGGSGGGGSEPKS | |
| | CDKTHTCPPCPAPELLGGPSVFLFPPKPKDTLMISRTPEVTCV | |
| | VVDVSHEDPEVKFNWYVDGVEVHNAKTKPREEQYNSTYRVVSV | |
| | LTVLHQDWLNGKEYKCKVSNKALPAPIEKTISKAKGQPREPQV | |
| | YTLPPSRDELTKNQVSLTCLVKGFYPSDIAVEWESNGQPENNY | |
| | KTTPPVLDSDGSFFLYSKLTVDKSRWQQGNVFSCSVMHEALHN | |
| | HYTQKSLSLSPGK | |
| CX201 | MDMRVPAQLLGLLLLWFPGSRCLTEEQLLGSLLRQLQLSEVPV | 129 |
| (L311E/ | LDRADMEKLVIPAHVRAQYVALLQRSHGDRSGGKGFSQSFREV | |
| P361D/ | AGRFLASEASTHLLVFGMEQRLPPNSELVQAVLRLFQEPVPKA | |
| R362Q) | ALHRHGGLSPRSAQARVTVEWLRVRDDGSNRTSLIDSRLVSVH | |
| | ESGWKAFDVTEAVNFWQQLSRPRQPLLLQVSVQREHLGPLASG | |
| | AHKLVRFASQGAPAGLGEPQLELHTLDLRDYGAQGDCDPEAPM | |
| | TEGTRCCRQEMYIDLQGMKWAKNWVLEPPGFLAYECVGTCQQP | |
| | PEALAFNWPFEGPRQCIASETASLPMIVSIKEGGRTRPQVVSL | |
| | PNMRVQKCSCASDGALVDQRLQPSGGGGSGGGGSGGGGSEPKS | |
| | CDKTHTCPPCPAPELLGGPSVFLFPPKPKDTLMISRTPEVTCV | |
| | VVDVSHEDPEVKFNWYVDGVEVHNAKTKPREEQYNSTYRVVSV | |
| | LTVLHQDWLNGKEYKCKVSNKALPAPIEKTISKAKGQPREPQV | |
| | YTLPPSRDELTKNQVSLTCLVKGFYPSDIAVEWESNGQPENNY | |
| | KTTPPVLDSDGSFFLYSKLTVDKSRWQQGNVFSCSVMHEALHN | |
| | HYTQKSLSLSPGK | |

For the thrombin site variant, an antibody-derived MDMRVPAQLLGLLLLWFPGSRC (UniProt: A0A0C4DH73; SEQ ID NO: 132) sequence was used as a signal sequence.

### Example 2-2: Transient Expression and Purification

The constructed expression vectors were transfected and the protein were expressed and purified in the same manner as Example 1, and then the expression level and purity thereof were analyzed. The results are shown in Tables 9 to 12 below.

The 42-LFc fusion protein used as a basis for construction of the variants showed an expression level of about 2.8 μg/ml, and showed an average protein purity of about 64.3% as measured by SE-HPLC after the first affinity purification. The Lefty A variant obtained by substituting the propeptide domain with LFB showed an about 7.5-fold increase in expression, and showed an average protein purity of about 83% as measured by SE-HPLC. The substitution of V63A for the propeptide domain of human Lefty A increased the expression level by about 2-fold, and the double substitution of V63A/R66Q increased the expression by about 2.3-fold (Table 9).

TABLE 9
Expression level and purity of human Lefty A
fusion protein variants (propeptide domain variants)

| | Expression | | |
|---|---|---|---|
| | level | Expression | SE-HPLC |
| Protein names | (μg/ml) | level fold | Purity (%) |
| 42 LFc | 2.8 | 1.0 | 64.3 |
| LFB 42 LFc | 21.0 | 7.5 | 83.2 |
| 42 LFc (V63A) | 5.2 | 1.9 | 81.7 |
| 42 LFc (R66Q) | 1.8 | 0.6 | 65.3 |
| 42 LFc (V63A/R66Q) | 6.5 | 2.3 | 78.2 |
| 42 LFc (V63A/R67H) | 6.2 | 2.2 | 65.8 |
| 42 LFc (V63A/ | 5.5 | 2.0 | 72.9 |
| R66Q/R67H) | | | |

Sequence mutations were introduced at the processing sites, and as a result, the protein purity measured by SE-HPLC was 84.1% in 42 LFc V2, and the expression level of 42 LFc V2 was similar to that before the mutations were introduced. 42 LFc V6 showed the highest protein purity of 90.9%, but the expression level thereof decreased to about 60% relative to that before the mutations were introduced (Table 10).

TABLE 10
Expression level and purity of human Lefty A
fusion protein variants (processing site variants)

| Protein | Expression | Expression | SE-HPLC |
|---|---|---|---|
| names | level (μg/ [symbol] ) | level fold | Purity ( % ) |
| 42 LFc | 2.8 | 1.0 | 64.3 |
| 42 LFc V1 | 1.6 | 0.6 | 71.6 |
| 42 LFc V2 | 2.3 | 0.8 | 84.1 |
| 42 LFc V4 | 1.6 | 0.6 | 63.6 |
| 42 LFc V6 | 1.8 | 0.6 | 90.9 |
| 42 LFc V7 | 1.7 | 0.6 | — |
| 42 LFc V10 | 1.5 | 0.5 | — |
| 42 LFc V11 | 1.9 | 0.7 | 79.9 |
| 42 LFc V12 | 2.4 | 0.9 | 81.4 |
| 42 LFc V13 | 3.0 | 1.1 | 84.3 |

The human Lefty A fusion protein variants (combination) were analyzed, and as a result, CX196 obtained using the LFB propeptide showed an about 1.8-fold increase in expression level and an increase in protein purity to 94.1%, and CX201 obtained using the human propeptide showed an about 1.9-fold increase in expression level and an increase in protein purity to 91.3% (Table 11 and FIG. 4).

TABLE 11
Expression level and purity of human Lefty A
fusion protein variants (combination)

| Protein | Expression | Expression | SE-HPLC |
|---|---|---|---|
| names | level (μg/ [symbol] ) | level fold | Purity |
| 42 LFc | 2.8 | 1.0 | 64.3 |
| CX196 | 5.1 | 1.8 | 94.1 |
| CX197 | 5.1 | 1.8 | 88.2 |
| CX201 | 5.2 | 1.9 | 91.3 |
| CX206 | 5.9 | 2.1 | 90.6 |
| CX207 | 6.1 | 2.2 | 86.8 |
| CX208 | 6.1 | 2.2 | 85.5 |

The human Lefty A fusion protein variants (thrombin cleavage site) were analyzed, and as a result, the P361S single mutant showed an about 1.5-fold increase in expression level. The double mutants of L311D/R362Q and L311E/R362Q and the triple mutants of L311D/P361D/R362Q and L311E/P361D/R362Q all showed a protein purity of about 95% (corresponding to an about 9% increase) together with a 1.5-fold increase in expression level (Table 12).

TABLE 12
Expression level and purity of human Lefty A
fusion protein variants (thrombin cleavage site)

| | Expression | | |
|---|---|---|---|
| | level | Expression | SE-HPLC |
| Protein names | (μg/ [symbol] ) | level fold | Purity (%) |
| CX201 | 6.9 | 1.0 | 85.9 |
| CX201 (L311D) | 8.6 | 1.2 | 91.7 |
| CX201 (L311E) | 7.8 | 1.1 | 91.8 |
| CX201 (P313D) | 6.4 | 0.9 | 88.6 |
| CX201 (P313E) | 7.1 | 1.0 | 84.9 |
| CX201 (P313S) | 6.5 | 0.9 | 79.9 |
| CX201 (R314K) | 7.2 | 1.0 | 91.1 |
| CX201 (R314Q) | 6.4 | 0.9 | 92.3 |
| CX201 (L359D) | 5.9 | 0.9 | 88.0 |
| CX201 (L359E) | 6.3 | 0.9 | 87.9 |
| CX201 (P361D) | 6.1 | 0.9 | 90.6 |
| CX201 (P361E) | 6.9 | 1.0 | 86.7 |
| CX201 (P361S) | 10.1 | 1.5 | 84.5 |
| CX201 (R362K) | 9.1 | 1.3 | 85.4 |
| CX201 (R362Q) | 9.5 | 1.4 | 87.5 |
| CX201 (L311D/ | 10.1 | 1.5 | 95.4 |
| R362Q) | | | |
| CX201 (L311E/ | 10.8 | 1.6 | 96.6 |
| R362Q) | | | |
| CX201 (L311D/ | 10.1 | 1.5 | 95.3 |
| P361D/R362Q) | | | |
| CX201 (L311E/ | 10.7 | 1.5 | 95.6 |
| P361D/R362Q) | | | |

### Example 3: Improvement in Stability in Serum

Using the human Lefty A fusion protein variants (thrombin cleavage site) constructed in Example 2, an in vitro serum stability test was performed. Each of the protein variants was diluted in mouse serum or plasma to a final concentration of 10 μg/ml, incubated at 37° C. for 4 hours, and then analyzed by Sandwich ELISA. The stability in mouse serum was analyzed by measuring the relative remaining amount of each human Lefty A variant in the serum under in vitro conditions, and the relative stability of each variant relative to CX201 is shown in Table 13 below. It could be confirmed that when the putative thrombin site mutation such as L311D, L311E, P361D or R362Q occurred, the stability of the variant in serum increased.

TABLE 13
Relative stability in serum after incubation
at 37° C. for 4 hours

| Protein samples | Relative stability in serum |
|---|---|
| CX201 (L311D) | 1.31 |
| CX201 (L311E) | 1.32 |
| CX201 (P313D) | 1.12 |
| CX201 (P313E) | 0.97 |
| CX201 (P313S) | 0.90 |
| CX201 (R314K) | 1.03 |
| CX201 (R314Q) | 1.08 |
| CX201 (L359D) | 1.19 |
| CX201 (L359E) | 1.18 |
| CX201 (P361D) | 1.23 |
| CX201 (P361E) | 1.13 |
| CX201 (P361S) | 0.83 |
| CX201 (R362K) | 1.17 |
| CX201 (R362Q) | 1.27 |
| CX201 (L311D/R362Q) | 1.13 |
| CX201 (L311E/R362Q) | 1.22 |
| CX201 (L311D/ | 1.15 |
| P361D/R362Q) | |
| CX201 (L311E/ | 1.16 |
| P361D/R362Q) | |
| CX201 | 1.00 |

### Example 4: Measurement of Binding Affinity for Nodal

The binding affinity between human Nodal protein and the human Lefty A fusion protein variant was measured using BIAcore.

Specifically, 50 RU of human Nodal (R&D systems, 3218-ND-025/CF) protein was fixed to a CM5 sensor chip. The human Lefty A fusion protein and their variants were diluted to concentrations of 500, 250, 125, 62.5 and 31.25 nM and injected sequentially from lower concentrations. Next, each dilution was associated by injection at a flow rate of 30 μl/min for 3 minutes and dissociated using running buffer for 5 minutes. The chip was regenerated using 15 μl of 50 mM NaOH. The association and dissociation rates for each cycle were evaluated using the “Bivalent analyte” model in BIAevaluation software version 4.1, and the BIAcore data are summarized in Table 14 below.

TABLE 14
Binding affinities of Lefty A fusion protein
and its variants for human Nodal

| | | | | | KD |
|---|---|---|---|---|---|
| | ka1 | kd1 | ka2 | kd2 | (nM) |
| 42 LFc | 1.18 × 10⁴ | 8.6 × 10⁻⁴ | 8.19 × | 4.46 × | 72.9 |
| | | | 10⁻³ | 10⁻² | |
| CX197 | 3.11 × 10² | 1.66 × 10⁻³ | 2.77 × | 2.3 × | 533.8 |
| | | | 10⁻⁴ | 10⁻⁵ | |
| CX201 | 8.71 × 10² | 1.08 × 10⁻³ | 5.46 × | 3.41 × | 124 |
| | | | 10⁻³ | 10⁻² | |

### Example 5: Prevention of ER Stress-Induced Apoptosis of Schwann Cells by Human Lefty A Protein

In this study, in order to identify a substance for treating hereditary peripheral neuropathy, a Schwann cell model was developed using an endoplasmic reticulum(ER) stress-inducing drug and used for drug identification.

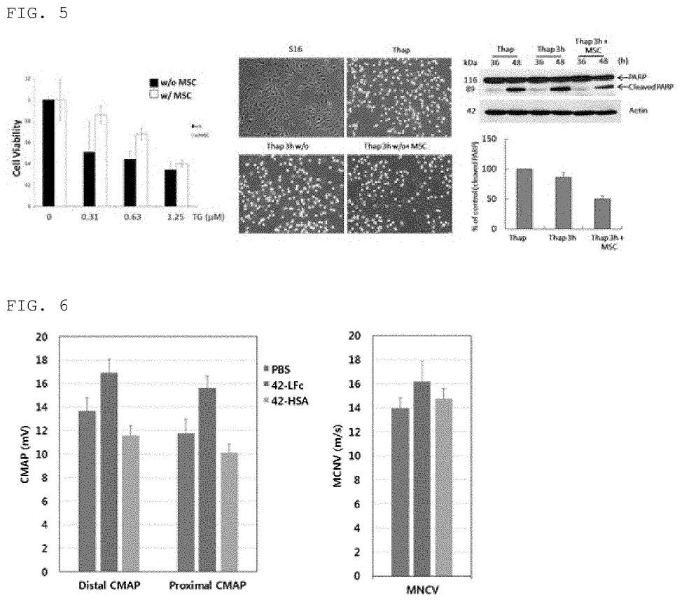

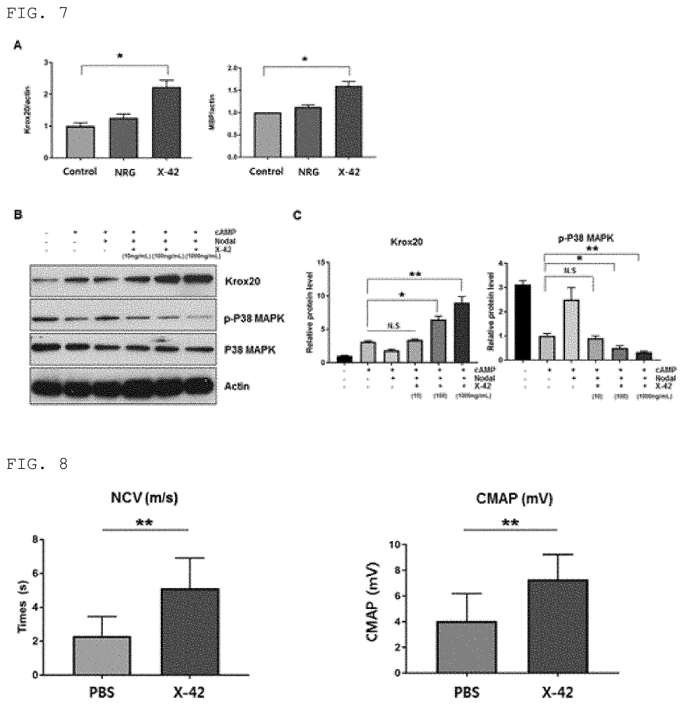

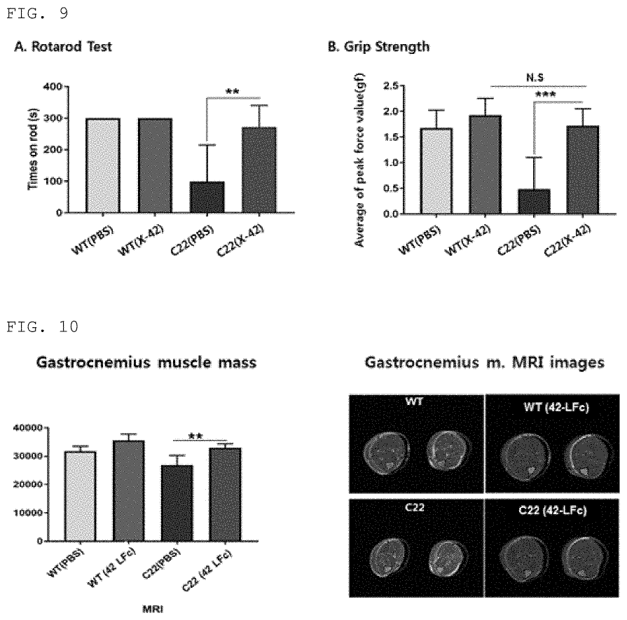

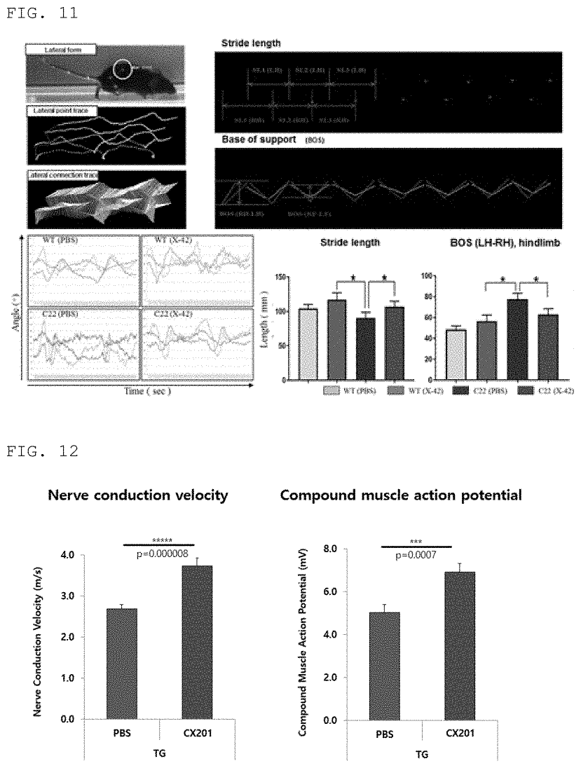

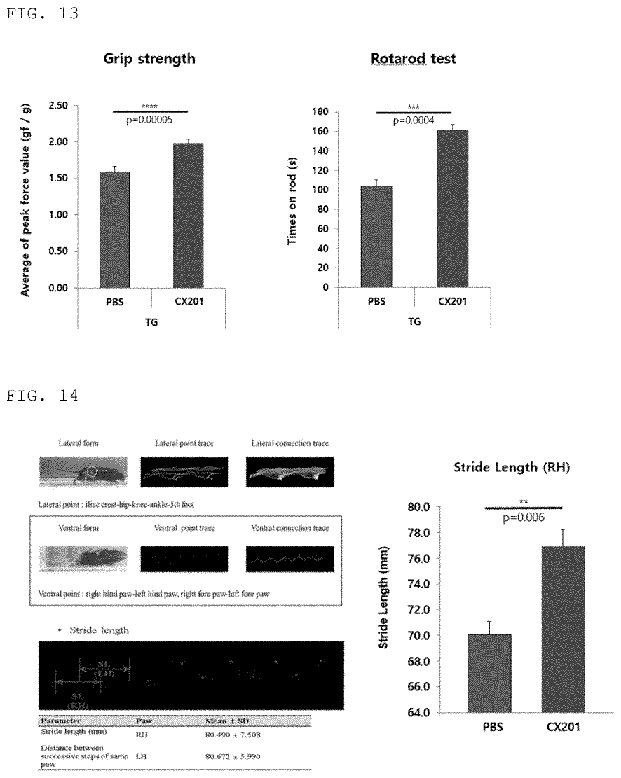

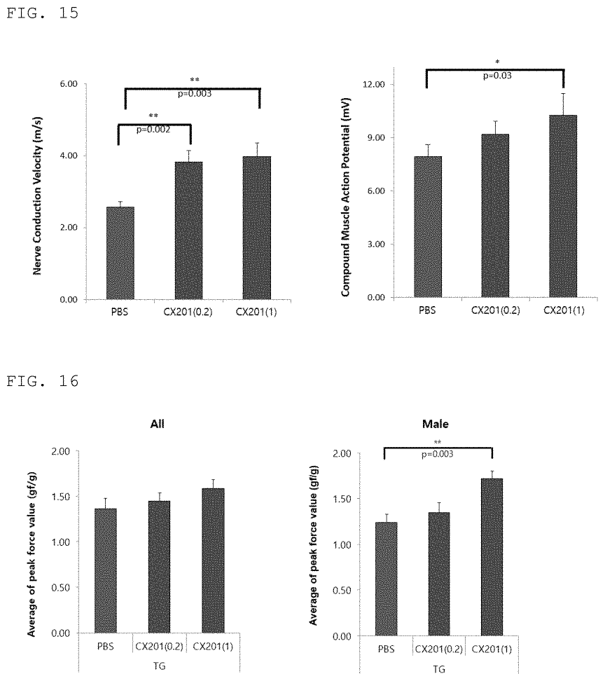

First, when mesenchymal stem cells were co-cultured in the endoplasmic reticulum stress cell model, cell death was effectively inhibited while the endoplasmic reticulum stress decreased. ER stress-induced apoptosis of Schwann cell was prevented when in co-culture with mesenchymal stem cells. Specifically, thapsigargin-induced ER stress was induced to S16 Schwann cells in a transwell chamber, and then umbilical cord-derived mesenchymal stem cells were co-cultured in the upper chamber, and the death of the S16 cells was analyzed. At this time, co-culture of the umbilical cord-derived mesenchymal stem cells inhibited the death of the S16 Schwann cells by 30% or more (FIG. 5).

From this result, it can be inferred that the paracrine factor secreted by umbilical cord stem cells inhibited the death of Schwann cells. Furthermore, a cytokine antibody array was performed to identify the therapeutic protein secreted from mesenchymal stem cells. The protein whose secretion from the umbilical cord stem cells during co-culture with the Schwann cells increased by two-fold or more was analyzed. In particular, it was observed that the secretion of the Lefty A protein effectively increased.

### Example 6: Improvement in Nerve Function by Human Lefty A Fusion Protein in Tr-J Mice

In order to examine whether the human Lefty A fusion protein would improve nerve function, electrophysiological study was performed using Trembler-J (Tr-J) mice, an animal model for CMT1 (Meekins et al., J Peripher Nery Syst 9(3):177-82 (2004)). Tr-J mice are spontaneously mutated mice in which a L16P mutation in the PMP22 gene occurred. The Tr-J mice have a phenotype of peripheral nerve demyelination and show decreases in nerve conduction velocity and compound muscle action potential (CMAP), as seen in CMT1 patients (Henry et al., J Neuropathol Exp Neurol 2(6):688-706 (1983); Valentijin et al., Nat Genet 2(4):288-911992 (1992)).

Tr-J mice were injected intraperitoneally with PBS or human Lefty A fusion proteins (1 μg/kg) every two days a total of 8 times from postnatal day 6 (p6) to postal day 20 (p20). On the day before nerve conduction study, the fur covering the hind limbs were shaved and depilated. The next day, the mice were anesthetized and the active recording needle electrode was placed at the gastrocnemius muscle and the reference electrode was placed just beneath the recording electrode in order to assess nerve conduction in sciatic nerve of the mice. The stimulus cathode was placed in the hip region 6 mm apart from the recording electrode, and the compound muscle action potential (CMAP) amplitudes and the motor nerve conduction velocity (MNCV) were measured using a Nicolet VikingQuest (Natus Medical, San Carlos, Calif.) device.

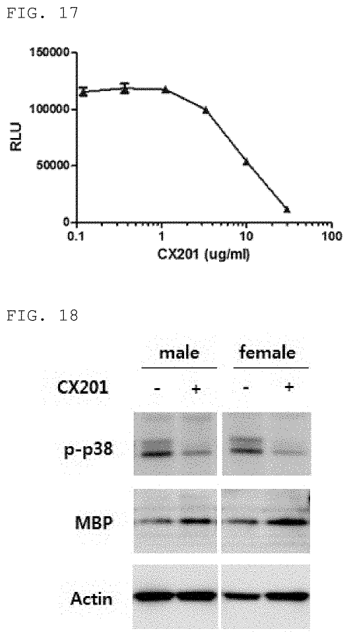

As shown in FIG. 6 and Table 15 below, the nerve conduction study showed that the 42 LFc fusion protein improved the nerve function of the Tr-J mice.

TABLE 15
Electrophysiological effects of Lefty A
fusion protein on nerve conduction in Tr-J mice

| | Distal CMAP | Proximal | MNCV |
|---|---|---|---|
| Drug | (mV) | CMAP (mV) | (m/s) |
| PBS | 13.7 ± 1.1 | 11.8 ± 1.2 | 13.9 ± 0.9 |
| 42-LFc | 16.9 ± 1.1* | 15.6 ± 1.1* | 16.2 ± 1.7 |
| 42-HSA | 11.6 ± 0.8 | 10.1 ± 0.7 | 14.7 ± 0.8 |

*p < 0.05

At a dose of 1 μg/kg, the LFc fusion human Lefty A protein significantly increased the compound muscle action potential (CMAP) and the motor nerve conduction velocity (MNCV) of the Tr-J mice compared to PBS or the HSA fusion protein. These data suggest that the human Lefty A fusion protein can be used to improve the nerve function of neuropathy patients.

### Example 7: Promotion of Schwann Cell Myelination by Human Lefty A Fusion Protein Variant

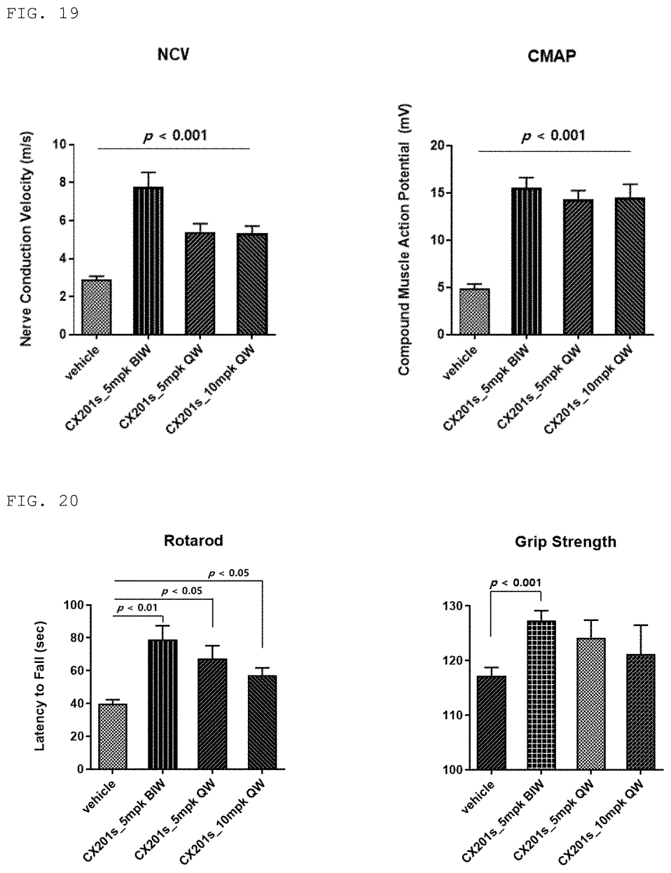

Treatment of the Lefty A fusion protein variant (42 LFc; X-42) on RT4-D6P2T Schwann cells resulted in increased expression of Krox20, an important transcriptional regulator of Schwann cell myelination, and myelin basic protein(MBP) whose expression is induced during myelination (FIG. 7A).

On the other hand, treatment of Nodal on RT4-D6P2T Schwann cells resulted in decreased expression of Krox20, and the inhibition of myelination by Nodal was recovered by the Lefty A fusion protein variant (FIGS. 7B and 7C). These results show that the Lefty A fusion variant promotes Schwann cell myelination, which is achieved by inhibiting nodal acting as a negative regulator of Schwann cell myelination.

### Example 8: Improvement in Nerve and Motor Functions by Intraperitoneal Injection (IP) of Human Lefty A Fusion Protein Variant (X-42) in C22 Mice (p6 Mice

C22 [strain of origin: (C57BL/6J×CBA/CA)F1] mice are transgenic mice carrying 7 copies of the human PMP22 gene and have a phenotype of severe peripheral neuron demyelination and have been widely studied as a Charcot-Marie-Tooth Disease type 1A (CMT1A) animal model (Robertson et al., J Anat 200(4):377-90 (2002); Norreel et al., Neuroscience 116(3):695-703 (2003)). The human Lefty A fusion protein variant(42 LFc; X−42) was administered intraperitoneally to the mice at a dose of 10 μg/kg a total of 10 times every two days from postnatal day 6 (p6) to postnatal day 24 (p24), and nerve conduction and muscle motor performance were assessed.

### Example 8-1: Nerve Conduction Study

On the day before nerve conduction study, the fur covering the hind limbs were shaved and depilated. The next day, the mice were anesthetized and the active recording needle electrode was placed at the gastrocnemius muscle and the reference electrode was placed just beneath the recording electrode in order to assess nerve conduction in sciatic nerve of the mice. The stimulus cathode was placed in the hip region 6 mm apart from the recording electrode, and the compound muscle action potential (CMAP) amplitudes and the motor nerve conduction velocity (MNCV) were measured using a Nicolet VikingQuest (Natus Medical, San Carlos, Calif.) device. The C22 mice showed significantly reduced MNCV and CMAP compared to wild type mice. On the other hand, the C22 mice administered with the human Lefty A fusion protein variant showed significantly increased MNCV and CMAP compared to the vehicle-administered group (FIG. 8).

### Example 8-2: Behavior Analysis

To evaluate motor function, rotarod test and hindlimb grip strength analysis were performed.

Specifically, the rotarod test was performed by placing a mouse on a 3 cm horizontal rotating rod (2 m/min) and then measuring the hold time. The mice were trained for 3 days before the test, and the test was performed four times per week. The latency to fall off the rotarod was recorded, with a maximum limit set at 5 min. As shown in FIG. 9A, locomotor performance of C22 mice was much lower than that of the wild-type mice. Administration of human Lefty A fusion protein variant significantly improved the motor performance of C22 mice in the rotarod test (FIG. 9A).

Grip strength in the hind-limbs of 3-4 weeks aged C22 mice was assessed using a grip strength meter. Mice were allowed to rest on the angled mesh assembly, facing away from the meter and with its hind limbs. As soon as the mouse grasped the metal grid or triangular pull bar with their hind toes, it was pulled directly toward the meter at the constant speed by the tail. The peak force was recorded in grams(g) by the device. C22 mice had a reduced hind-limb strength compared to wild type mice, and treatment of human Lefty A protein variant showed a significant therapeutic effect on the grip strength (FIG. 9B). The hindlimb grip strength in C22 mice administrated with the human Lefty A fusion protein variant increased approximately 3-fold (FIG. 9B).

### Example 9: An Increase in Muscle Mass by Intraperitoneal Injection (IP) of Human Lefty A Fusion Protein Variant (X-42) in C22 Mice (p6 Mice

The change in muscle mass by the human Lefty A fusion protein variant was examined using 6-day-old (p6) wild type mice or C22 mice. PBS or the human Lefty A fusion protein variant (10 μg/kg) was intraperitoneally administered to the mice at a dose of 10 μg/kg a total of 10 times every two days from postnatal day 6 (p6) to postnatal day 24 (p24), and then the gastrocnemius muscle area was measured using magnetic resonance imaging. The administration of the human Lefty A fusion protein variant showed a significant increase in muscle mass in the CMT1 model mice as well as the wild-type mice (FIG. 10).

### Example 10: Improvement in Gait by Intraperitoneal Injection (IP) of Human Lefty A Fusion Protein Variant (X-42) in C22 Mice (p6 Mice

In order to elucidate effect of the Lefty A fusion protein variant on gait of C22 mice, PBS or the human Lefty A fusion protein variant (10 μg/kg) was administered intraperitoneally to 6-day-old mice 10 times (every two days). After allowing the mice to cross a restricted path, gait was monitored, and hip, knee and ankle joint angles and two parameters (stride length and base of support (BOS)) were analyzed (FIG. 11). The stride length was calculated by tracking the right hind paw and the left hind paw, and the BOS value were determined by calculating the left and right of the stride. As a result, an improvement in pelvic stride length was observed in the mouse group administered with the Lefty A fusion protein variant (right of FIG. 11).

Compared to the wild-type mice, the C22 mice showed significant differences in the hip, knee and ankle joint angles due to gait abnormalities. However, the change in angle in the C22 mice was reduced when the lefty A fusion protein was administered in C22 mice (bottom left in FIG. 11). This result shows that the neuromuscular function was improved to some extent, but not at the level of wild-type mice.

### Example 11: Improvement in Nerve and Motor Functions by Intraperitoneal Injection (IP) of Human Lefty A Fusion Protein Variant (CX201) in C22 Mice (p6 Mice

C22 mice were injected intraperitoneally with the human Lefty A fusion protein variant CX201 at a dose of 0.2 mg/kg 10 times in total every two days. The injection started as early as at postnatal day 6 when myelination has not started even in normal conditions. Then, as described in Example 8 above, nerve conduction study and behavioral analysis were performed.

The C22 mice injected with the human Lefty A fusion protein variant CX201 showed significant increases in the motor nerve conduction speed (MNCV) and the compound muscle action potential (CMAP) compared to the vehicle-administered group (FIG. 12).

In addition, as shown in FIG. 13, CX201-injected C22 mice showed statistically significant improvements in hindlimb grip strength and motor performance on rotarod, compared with vehicle controls.

### Example 12: Effect on Gait by Intraperitoneal Injection (IP) of Human Lefty A Fusion Protein Variant (CX201) in C22 Mice (p6 Mice

In order to elucidate effect of the Lefty A fusion protein variant CX201 on the gait of C22 mice, PBS or the human Lefty A fusion protein variant (200 μg/kg) was administered intraperitoneally to 6-day-old mice 10 times (once every two days), and then gait analysis was performed as described in Example 10.

In particular, analysis of the stride length from one heel to the next heel showed that the gait of C22 mice administered with the Lefty A fusion protein variant was significantly improved (FIG. 14).

### Example 13: Improvement in Nerve and Motor Functions by Subcutaneous Injection of Human Lefty A Fusion Protein Variant in C22 Mice (p21 Mice

The human Lefty A fusion protein variant CX201 was subcutaneously injected into C22 mice on postnatal day 21 (p21) when myelination development is predominantly manifested, and as described in Example 8 above, nerve conduction study and behavioral analysis were performed.

The C22 mice administered with CX201 for 4 weeks showed significant increases in both the motor nerve conduction velocity and the compound muscle action potential compared to vehicle-administrated group (FIG. 15). In particular, the hindlimb grip strength increased in the male mice, with a statistical significance (FIG. 16). These data suggest that administration of the fusion protein variant CX201 at late stage of myelination differentiation as well as early postnatal stage can improve nerve and motor functions.

### Example 14: Inhibition of Myostatin Signaling by Human Lefty A Fusion Protein Variant

Reporter gene analysis was used to assess whether the human Lefty A fusion protein variant can inhibit myostatin signaling. Rhabdomyosarcoma A204 cells were transfected with the Smad2/3-reactive luciferase reporter vector pGL4.48 (Promega, USA), and then the cells stably transfected with the vector were selected through antibiotic selection. Myostatin was pre-incubated with increasing concentrations of the human Lefty A fusion protein variant for 45 minutes. After addition of the media, the cells were incubated for 6 hours, and then luciferase activity induced by myostatin was detected using bio-glo lucierase assay reagent (Promega, USA). Myostatin induced strong luciferase expression in the cell line stably introduced with the vector, and the human Lefty A fusion protein variant dose-dependently inhibited myostatin signaling (FIG. 17).

### Example 15: Inhibition of p38 Signaling by Human Lefty A Fusion Protein Variant

It is known that TGF-b family BMP7 suppresses the expression of myelin genes in and retards peripheral myelination by phosphorylation of p38 (Liu X et al., Sci Rep 6:31049 (2016)). Thus, it was investigated whether the human Lefty A fusion protein variant can inhibit p38 phosphorylation. Specifically, the human Lefty A fusion protein variant was administered to C22 mice as described in Example 7, phosphorylated p38 of the sciatic nerve was analyzed using Western blotting method.

As shown in FIG. 18, phosphorylation of p38 was inhibited and the expression of MBP protein increased in the sciatic nerve of the C22 mice administered with the human Lefty A fusion protein variant. These data show that the human Lefty A fusion protein variant can promote myelination by blocking p38 signaling, a negative regulator of myelination. Therefore, the human Lefty A fusion protein can be used as an agent for treating peripheral neuropathy, such as neurodegenerative disease caused by demyelination.

### Example 16: Construction of Cell Line Producing Human Lefty A Fusion Protein Variant

CHO—S(cGMP-banked) cells were inoculated into 30 mL of CD-FortiCHO medium in a 125-mL Erlenmeyer flask at a density of 1×10⁶ cells/mL. 50 μg of an expression vector inserted with the human Lefty A fusion protein variant (CX201s; comprising an antibody-derived MDMRVPAQLLGLLLLWFPGSRC sequence as a signal sequence in the CX201 fusion protein; human Lefty A linked to human IgG1 Fc via a SGGGGSGGGGSGGGGS linker; SEQ ID NO: 133 in Table 16) gene was placed in a 50 mL conical tube, and OptiPRO SFM was added to a final volume of 1.5 mL, followed by vortexing. 50 μL of Freestyle MAX reagent was placed in another 50 mL conical tube and OptiPRO SFM was added to a final volume of 1.5 mL, followed by vortexing. The Freestyle MAX solution was added to the DNA solution, and then allowed to stand at room temperature for 10 minutes. After 10 minutes, the Erlenmeyer flask containing the cells was treated with a DNA-Freestyle MAX reagent complex to transform the cells.

First screening and second screening were sequentially performed to induce gene amplification. A CX201s producing cell line was selected using ClonePix from the pool showing the highest expression level among the pools obtained after completion of the first and second screenings. The selected clones were seed-cultured for 6 days, and then suspended in freezing medium (CD-FortiCHO medium 90%+DMSO 10%) to a concentration of 1.0×10⁷ cells/mL, and 1 mL of the suspension was dispensed into each cryotube, thereby preparing RCB (research cell bank).

TABLE 16
Amino acid sequence of human Lefty A fusion
protein variant

| | | SEQ ID |
|---|---|---|
| | Amino acid sequence | NO: |
| CX201s | MDMRVPAQLLGLLLLWFPGSRCLTEEQLLGSLLRQLQLSEVPV | 133 |
| | LDRADMEKLVIPAHVRAQYVALLQRSHGDRSGGKGFSQSFREV | |
| | AGRFLASEASTHLLVFGMEQRLPPNSELVQAVLRLFQEPVPKA | |
| | ALHRHGGLSPRSAQARVTVEWLRVRDDGSNRTSLIDSRLVSVH | |
| | ESGWKAFDVTEAVNFWQQLSRPRQPLLLQVSVQREHLGPLASG | |
| | AHKLVRFASQGAPAGLGEPQLELHTLDLRDYGAQGDCDPEAPM | |
| | TEGTRCCRQEMYIDLQGMKWAKNWVLEPPGFLAYECVGTCQQP | |
| | PEALAFNWPFLGPRQCIASETASLPMIVSIKEGGRTRPQVVSL | |
| | PNMRVQKCSCASDGALVPRRLQPSGGGGSGGGGSGGGGSEPKS | |
| | CDKTHTCPPCPAPELLGGPSVFLFPPKPKDTLMISRTPEVTCV | |
| | VVDVSHEDPEVKFNWYVDGVEVHNAKTKPREEQYNSTYRVVSV | |
| | LTVLHQDWLNGKEYKCKVSNKALPAPIEKTISKAKGQPREPQV | |
| | YTLPPSREEMTKNQVSLTCLVKGFYPSDIAVEWESNGQPENNY | |
| | KTTPPVLDSDGSFFLYSKLTVDKSRWQQGNVFSCSVMHEALHN | |
| | HYTQKSLSLSPGK | |

The CX201 is a variant in which amino acid residue substitutions of V63A, R66Q, G132R and R135A in the sequence of 42 LFc occurred, and the CX203 is a variant in which amino acid residue substitutions of V63A, R66Q, G132R and G134E in the sequence of 42 LFc occurred.

### Example 17: Improvement in Nerve Motor Functions by Subcutaneous Injection of Human Lefty A Fusion Protein Variant in C22 Mice (p35 Mice

C22 mice at 5 weeks of age (p35), which have undergone myelination of the peripheral nerve, were administered subcutaneously with the human lefty A fusion protein variant CX201s, once or twice a week at a dose of 5 mg/kg or once a week at a dose of 10 mg/kg. Electrophysiological evaluation of nerve function was carried out as described in Example 8. The C22 mice administered with CX201s showed significant increases in both nerve conduction velocity (NCV) and compound muscle action potential (CMAP) compared to the vehicle-administered group (FIG. 19).

Rotarod test and grip strength analysis were performed to evaluate motor function. For rotarod test, a mouse was placed on a rod rotating at 12 rpm, and latency to fall off the rod was recorded, with a maximum limit set at 200 sec. As shown in FIG. 19, motor performance on the rotarod of the C22 mice injected with CX201s was significantly improved compared to the vehicle controls. In addition, the whole-limb grip strength of the mice was measured using a grip strength meter to evaluate the neuromuscular function of the mice. When C22 mice were injected with CX201s twice a week at a dose of 5 mg/kg, the whole-limb grip strength was statistically significantly improved (FIG. 20).

Taken together, subcutaneous injection of the human Lefty A fusion protein variant CX201s to 5-week-old C22 mice for four weeks or more improved the nerve conduction and motor function of the C22. In particular, administration of CX201s at a dose of 5 mg/kg twice a week was most effective.

### Example 18: Inhibition of Nodal Signaling by Human Lefty A Fusion Protein Variant

Nodal, a member of the TGF-β family, activates Smad signaling. Once Nodal binds to activin receptors, Smad2 and Smad3 are phosphorylated, bind to Smad4, move into the nucleus, and then regulate transcription of various genes. Effect of human Lefty A fusion protein variant on the Nodal-induced Smad signaling was evaluated using a Nodal-responsive P19 mouse embryonic cancer cell line.

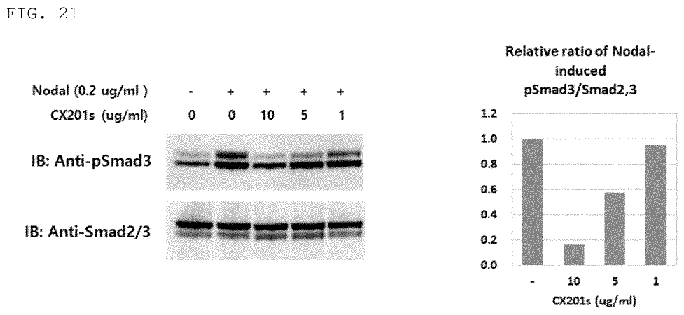

Nodal was pre-incubated with various concentrations of the human Lefty A fusion protein variant CX201s for 30 minutes. Cells were treated with Nodal alone or Nodal-Lefty A fusion protein variant for 1 hour, and then Smad3 phosphorylation induced by Nodal was evaluated using the cell lysates by Western blot analysis. Treatment of the cells with the Nodal protein alone induced strong Smad3 phosphorylation, which was dose-dependently inhibited by the human Lefty A fusion protein variant (FIG. 21).

### INDUSTRIAL APPLICABILITY

According to the present invention, a human Lefty A protein variant and a fusion protein comprising the variant are constructed, which have better stability than naturally occurring human Lefty A protein, and thus are expressed at high levels and produced in high yield in animal cells. In addition, administration of the constructed human Lefty A protein variant or fusion protein can restore the nerve and motor functions of animal models of peripheral neuropathy. Accordingly, the use of the human Lefty A protein variant or fusion protein can effectively prevent or treat various nerve diseases and muscle diseases.

Although the present invention has been described in detail with reference to the specific features, it will be apparent to those skilled in the art that this description is only for a preferred embodiment and does not limit the scope of the present invention. Thus, the substantial scope of the present invention will be defined by the appended claims and equivalents thereof.

1. A human Lefty A protein variant comprising the amino acid sequence of L22 to P366 of a human Lefty A protein having the amino acid sequence of SEQ ID NO: 131, the human Lefty A protein variant comprising:
(1) a substitution of one or more amino acid residues at processing sites (R74 to R77 and R132 to R135); and
(2) a substitution of one or more amino acid residues in a propeptide domain (L22 to S73).

2. The human Lefty A protein variant of claim 1, wherein the substitution of amino acid residues in the propeptide domain (L22 to S73) is a substitution of amino acid residues at one or more positions selected from the group consisting of E24, L27, R33, S38, V40, V42, R45, M48, K50, A55, V63, R66, R67, G70 and D71.

3. The human Lefty A protein variant of claim 2, wherein the substitution of amino acid residues in the propeptide domain (L22 to S73) is one or more amino acid residue substitutions selected from the group consisting of E24G, S38K, V42T, K50E, A55T, V63A and R66Q.

4. The human Lefty A protein variant of claim 3, wherein the substitution of amino acid residues in the propeptide domain (L22 to S73) comprises V63A, and further comprises one or more amino acid residue substitutions selected from the group consisting of E24G, S38K, V42T, K50E, A55T and R66Q.

5. The human Lefty A protein variant of claim 1, wherein the substitution of one or more amino acid residues at the processing sites (R74 to R77 and R132 to R135) is one or more amino acid residue substitutions selected from the group consisting of R74G, R77G, R77V, R132G and R135G.

6. The human Lefty A protein variant of claim 5, wherein the amino acid sequence of positions R74 to R77 is RGKR (SEQ ID NO: 180), GGKG (SEQ ID NO: 188), RGKA (SEQ ID NO: 189), RGKV (SEQ ID NO: 190) or RHGG (SEQ ID NO: 183), and the amino acid sequence of positions R132 to R135 is RHGR (SEQ ID NO: 181), GHGR (SEQ ID NO: 182), RHGG (SEQ ID NO: 183), RHER (SEQ ID NO: 184), GHGG (SEQ ID NO: 185), RHGA (SEQ ID NO: 186) or RHGV (SEQ ID NO: 187) which result from the substitution of one or more amino acid residues at the processing sites (R74 to R77 and R132 to R135).

7. The human Lefty A protein variant of claim 1, further comprising a substitution of one or more amino acid residues at a thrombin cleavage site (L311, P313, R314, L359, P361 or R362).

8. The human Lefty A protein variant of claim 7, wherein the amino acid residues at one or more positions selected from the group consisting of the thrombin cleavage sites L311, P313, R314, L359, P361 and R362 are substituted with amino acid residues selected from the group consisting of aspartic acid (D), glutamic acid (E), serine (S), lysine (K) and glutamine (Q).

9. The human Lefty A protein variant of claim 1, further comprising a substitution of one or more amino acid residues at a fragmentation site (S202 or S223) with amino acid residues other than serine (S) and cysteine (C).

10. The human Lefty A protein variant of claim 1, further comprising a signal peptide at the N-terminus.

11. A fusion protein comprising the human Lefty A protein variant of claim 1.

12. The fusion protein of claim 11, wherein the fusion protein in which the human Lefty A protein variant is fused with Fc or albumin.

13. The fusion protein of claim 12, wherein the fusion protein in which Fc or albumin fused at the C-terminus of the human Lefty A protein variant.

14. The fusion protein of claim 12, wherein the human Lefty A protein variant and Fc or albumin are fused together via a linker.

15. The fusion protein of claim 11, wherein the fusion protein has any one amino acid sequence selected from the group consisting of amino acid sequences set forth in SEQ ID NOS: 134 to 178.

16. A nucleic acid molecule encoding the fusion protein of claim 11.

17. An expression vector comprising the nucleic acid molecule of claim 16.

18. A recombinant cell into which the expression vector of claim 17 has been introduced.

19. A method of producing a fusion protein comprising a human Lefty A protein variant using the recombinant cell of claim 18.

20. A composition for preventing and/or treating neuromuscular disease, which comprises either the human Lefty A protein variant of claim 1 or a fusion protein comprising the human Lefty A protein variant.

21. The composition for preventing and/or treating neuromuscular disease of claim 20, wherein the neuromuscular disease is a Nodal and/or myostatin signaling-related disease.

22. The composition for preventing and/or treating neuromuscular disease of claim 21, wherein the Nodal and/or myostatin signaling-related disease is myopathy, peripheral neuropathy, or rigid spine syndrome.

23. The composition for preventing and/or treating neuromuscular disease of claim 22, wherein the myopathy is selected from the group consisting of sarcopenia, muscular dystrophy, myasthenia gravis, amyotrophic lateral sclerosis (or Lou Gehrig's disease), primary lateral sclerosis, progressive muscular atrophy, Kennedy's disease (or spinobulbar muscular atrophy), spinal muscular atrophy and distal myopathy.

24. The composition for preventing and/or treating neuromuscular disease of claim 22, wherein the peripheral neuropathy is selected from the group consisting of Charcot-Marie-Tooth disease, chronic inflammatory demyelinating polyneuropathy, carpal tunnel syndrome, diabetic peripheral neuropathy and Guillain-Barre syndrome.
